(19) United States
(12) Patent Application Publication
(10) Pub. No.: US 2014/0179664 A1
(43) Pub. Date: Jun. 26, 2014

(54) Heterocyclyl Pyrimidine Analogues As JAK Inhibitors

(71) Applicants: Jay Freeman, Norwich (GB); Valerie Reader, Linton (GB); Glynn Addison, Babraham (GB); Nigel Ramsden, Royston (GB); Jane Elizabeth Scanlon, Sawston (GB); Richard John Harrison, Cambridge (GB)

(72) Inventors: Jay Freeman, Norwich (GB); Valerie Reader, Linton (GB); Glynn Addison, Babraham (GB); Nigel Ramsden, Royston (GB); Jane Elizabeth Scanlon, Sawston (GB); Richard John Harrison, Cambridge (GB)

(73) Assignee: Cellzome Limited, Brentford, Middlesex (GB)

(21) Appl. No.: 14/234,662

(22) PCT Filed: Jul. 24, 2012
(86) PCT No.: PCT/EP2012/064515

§ 371 Date: Feb. 19, 2014

Related U.S. Application Data
(60) Provisional application No. 61/635,921, filed on Apr. 20, 2012.

(30) Foreign Application Priority Data
Jul. 28, 2011 (EP) 11175807.4

(51) Int. Cl. C07D 491/107; C07D 403/14; C07D 413/14; C07D 403/12

(52) U.S. Cl. CPC C07D 491/107; C07D 403/12; C07D 403/14; C07D 413/14
USPC 514/210.2; 544/324; 514/275; 544/122; 514/235.8

(57) ABSTRACT

The present invention relates to compounds of formula (I)

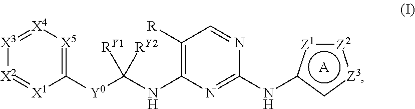

wherein X¹ to X⁵, Z¹ to Z³, Y⁰, R^{Y1}, R^{Y2} and R have the meaning as cited in the description and the claims. Said compounds are useful as JAK inhibitors for the treatment or prophylaxis of immunological, inflammatory, autoimmune, allergic disorders, and immunologically-mediated diseases. The invention also relates to pharmaceutical compositions including said compounds, the preparation of such compounds as well as the use as medicaments.

The present invention relates to a novel class of kinase inhibitors, including pharmaceutically acceptable salts, prodrugs and metabolites thereof, which are useful for modulating protein kinase activity for modulating cellular activities such as signal transduction, proliferation, and cytokine secretion. More specifically the invention provides compounds which inhibit, regulate and/or modulate kinase activity, in particular JAK3 activity, and signal transduction pathways relating to cellular activities as mentioned above. Furthermore, the present invention relates to pharmaceutical compositions comprising said compounds, for example for the treatment or prevention of an immunological, inflammatory, autoimmune, or allergic disorder or disease or a transplant rejection or a Graft-versus host disease and processes for preparing said compounds.

Kinases catalyze the phosphorylation of proteins, lipids, sugars, nucleosides and other cellular metabolites and play key roles in all aspects of eukaryotic cell physiology. Especially, protein kinases and lipid kinases participate in the signaling events which control the activation, growth, differentiation and survival of cells in response to extracellular mediators or stimuli such as growth factors, cytokines or chemokines. In general, protein kinases are classified in two groups, those that preferentially phosphorylate tyrosine residues and those that preferentially phosphorylate serine and/or threonine residues. The tyrosine kinases include membrane-spanning growth factor receptors such as the epidermal growth factor receptor (EGFR) and cytosolic non-receptor kinases such as Janus kinases (JAK).

Inappropriately high protein kinase activity is involved in many diseases including cancer, metabolic diseases, autoimmune or inflammatory disorders. This effect can be caused either directly or indirectly by the failure of control mechanisms due to mutation, overexpression or inappropriate activation of the enzyme. In all of these instances, selective inhibition of the kinase is expected to have a beneficial effect.

One group of kinases that has become a recent focus of drug discovery is the Janus kinase (JAK) family of non-receptor tyrosine kinases. In mammals, the family has four members, JAK1, JAK2, JAK3 and Tyrosine kinase 2 (TYK2). Each protein has a kinase domain and a catalytically inactive pseudo-kinase domain. The JAK proteins bind to cytokine receptors through their amino-terminal FERM (Band-4.1, ezrin, radixin, moesin) domains. After the binding of cytokines to their receptors, JAKs are activated and phosphorylate the receptors, thereby creating docking sites for signalling molecules, especially for members of the signal transducer and activator of transcription (Stat) family (Yamaoka et al., 2004. The Janus kinases (Jaks). Genome Biology 5(12): 253).

In mammals, JAK1, JAK2 and TYK2 are ubiquitously expressed. By contrast, the expression of JAK3 is predominantly in hematopoietic cells and it is highly regulated with cell development and activation (Musso et al., 1995. 181(4):1425-31).

The study of JAK-deficient cell lines and gene-targeted mice has revealed the essential, nonredundant functions of JAKs in cytokine signalling. JAK1 knockout mice display a perinatal lethal phenotype, probably related to the neurological effects that prevent them from sucking (Rodig et al., 1998. Cell 93(3):373-83). Deletion of the JAK2 gene results in embryonic lethality at embryonic day 12.5 as a result of a defect in erythropoiesis (Neubauer et al., 1998. Cell 93(3):397-409). Interestingly, JAK3 deficiency was first identified in humans with autosomal recessive severe combined immunodeficiency (SCID) (Macchi et al., 1995. Nature 377(6544):65-68). JAK3 knockout mice too exhibit SCID but do not display non-immune defects, suggesting that an inhibitor of JAK3 as an immunosuppressant would have restricted effects in vivo and therefore presents a promising drug for immunosuppression (Papageorgiou and Wikman 2004, Trends in Pharmacological Sciences 25(11):558-62).

Activating mutations for JAK3 have been observed in acute megakaryoblastic leukemia (AMKL) patients (Walters et al., 2006. Cancer Cell 10(1):65-75). These mutated forms of JAK3 can transform Ba/F3 cells to factor-independent growth and induce features of megakaryoblastic leukemia in a mouse model.

Diseases and disorders associated with JAK3 inhibition are further described, for example in WO 01/42246 and WO 2008/060301.

Several JAK3 inhibitors have been reported in the literature which may be useful in the medical field (O'Shea et al., 2004. Nat. Rev. Drug Discov. 3(7):555-64). A potent JAK3 inhibitor (CP-690,550) was reported to show efficacy in an animal model of organ transplantation (Changelian et al., 2003, Science 302(5646):875-888) and clinical trials (reviewed in: Pesu et al., 2008. Immunol. Rev. 223, 132-142). The CP-690,550 inhibitor is not selective for the JAK3 kinase and inhibits JAK2 kinase with almost equipotency (Jiang et al., 2008, J. Med. Chem. 51(24):8012-8018). It is expected that a selective JAK3 inhibitor that inhibits JAK3 with greater potency than JAK2 may have advantageous therapeutic properties, because inhibition of JAK2 can cause anemia (Ghoreschi et al., 2009. Nature Immunol. 4, 356-360).

Pyrimidine derivatives exhibiting JAK3 and JAK2 kinase inhibiting activities are described in WO-A 2008/009458. Pyrimidine compounds in the treatment of conditions in which modulation of the JAK pathway or inhibition of JAK kinases, particularly JAK3 are described in WO-A 2008/118822 and WO-A 2008/118823.

Fluoro substituted pyrimidine compounds as JAK3 inhibitors are described in WO-A 2010/118986. Heterocyclyl pyrazolopyrimidine analogues as JAK inhibitors WO-A 2011/048082.

WO-A 2008/129380 relates to sulfonyl amide derivatives for the treatment of abnormal cell growth. WO-A 2006/117560 and J. of Molecular Graphics and Modelling (29) 2010, 309-320 describe pyrazolylamino-substituted pyrimidines and their use in the treatment of cancer. EP 1 054 004 A1 describes pyrimidine derivatives and their use in inflammation.

Even though JAK inhibitors are known in the art there is a need for providing additional JAK inhibitors having at least partially more effective pharmaceutically relevant properties, like activity, selectivity especially over JAK2 kinase, and ADME properties.

Thus, an object of the present invention is to provide a new class of compounds as JAK inhibitors which preferably show selectivity over JAK2 and may be effective in the treatment or prophylaxis of disorders associated with JAK.

Accordingly, the present invention provides compounds of formula (I)

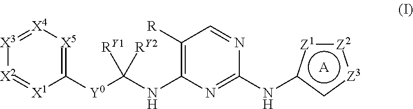

or a pharmaceutically acceptable salt or isotopic derivative thereof, wherein

R is H; F; Cl; Br; CN; CH₃; CF₃ or C(O)NH₂;

Ring A is a 5 membered aromatic heterocycle in which Z¹, Z², and Z³ are independently selected from the group consisting of C(R¹); N; and N(R¹), provided that at least one of Z¹, Z², Z³ is N or N(R¹);

Each R¹ is independently H, halogen; CN; C(O)OR²; OR²; C(O)R²; C(O)N(R²R^{2a}); S(O)₂N(R²R^{2a}); S(O)N(R²R^{2a}); S(O)₂R²; S(O)R²; N(R²)S(O)₂N(R^{2a}R^{2b}); N(R²)S(O)N(R^{2a}R^{2b}); SR²; N(R²R^{2a}); NO₂; OC(O)R²; N(R²)C(O)R^{2a}; N(R²)S(O)₂R^{2a}; N(R²)S(O)R^{2a}; N(R²)C(O)N(R^{2a}R^{2b}); N(R²)C(O)OR^{2a}; OC(O)N(R²R^{2a}); T¹; C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R³, which are the same or different;

R², R^{2a}, R^{2b} are independently selected from the group consisting of H; T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R³, which are the same or different;

R³ is halogen; CN; C(O)OR⁴; OR⁴; C(O)R⁴; C(O)N(R⁴R^{4a}); S(O)₂N(R⁴R^{4a}); S(O)N(R⁴R^{4a}); S(O)₂R⁴; S(O)R⁴; N(R⁴)S(O)₂N(R^{4a}R^{4b}); N(R⁴)S(O)N(R^{4a}R^{4b}); SR⁴; N(R⁴R^{4a}); NO₂; OC(O)R⁴; N(R⁴)C(O)R^{4a}; N(R⁴)S(O)₂R^{4a}; N(R⁴)S(O)R^{4a}; N(R⁴)C(O)N(R^{4a}R^{4b}); N(R⁴)C(O)OR^{4a}; OC(O)N(R⁴R^{4a}); or T¹;

R⁴, R^{4a}, R^{4b} are independently selected from the group consisting of H; T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁵, which are the same or different;

R⁵ is halogen; CN; C(O)OR^{5a}; OR^{5a}; C(O)R^{5a}; C(O)N(R^{5a}R^{5b}); S(O)₂N(R^{5a}R^{5b}); S(O)N(R^{5a}R^{5b}); S(O)₂R^{5a}; S(O)R^{5a}; N(R^{5a})S(O)₂N(R^{5b}R^{5c}); N(R^{5a})S(O)N(R^{5b}R^{5c}); SR^{5a}; N(R^{5a}R^{5b}); NO₂; OC(O)R^{5a}; N(R^{5a})C(O)R^{5a}; N(R^{5a})S(O)₂R^{5b}; N(R^{5a})S(O)R^{5b}; N(R^{5a})C(O)N(R^{5b}R^{5c}); N(R^{5a})C(O)OR^{5b}; or OC(O)N(R^{5a}R^{5b}); R^{5a}, R^{5b}, R^{5c} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

T¹ is C₃₋₇ cycloalkyl; saturated 4 to 7 membered heterocyclyl; or 7 to 11 membered heterobicyclyl, wherein T¹ is optionally substituted with one or more R¹⁰, which are the same or different;

Y⁰ is (CR^{Y3}R^{Y4})ₙ;

n is 0; or 1;

One of R^{Y1}; R^{Y2}; R^{Y3}; R^{Y4} is R^{Y0} and the others are selected from the group consisting of H; CH₃; and CF₃;

R^{Y0} is unsubstituted C₁₋₄ alkyl; CH₂CH₂OR^{Y5}; CH₂CH₂C(O)T^{Y1}; CH₂CH₂C(O)OR^{Y5}; CH₂CH₂OC(O)R^{Y5}; CH₂CH₂N(R^{Y5}R^{Y5a}); CH₂CH₂N(R^{Y5})C(O)R^{Y5a}; CH₂CH₂C(O)N(R^{Y5}R^{Y5a}); CH₂OR^{Y5}; CH₂C(O)T^{Y1}; CH₂C(O)OR^{Y5}; CH₂OC(O)R^{Y5}; CH₂N(R^{Y5}R^{Y5a}); CH₂N(R^{Y5})C(O)R^{Y5a}; CH₂C(O)N(R^{Y5}R^{Y5a}); C(O)T^{Y1}; C(O)OR^{Y5}; or C(O)N(R^{Y5}R^{Y5a});

R^{Y5}, R^{Y5a} are independently selected from the group consisting of H; T^{Y1}; and C₁₋₄ alkyl, wherein C₁₋₄ alkyl is optionally substituted with one or more R^{Y6}, which are the same or different;

R^{Y0} is halogen; OR^{Y7}; C(O)T^{Y1}; C(O)OR^{Y7}; OC(O)R^{Y7}; N(R^{Y7}R^{Y7a}); or N(R^{Y7})C(O)R^{Y7a};

R^{Y7}; R^{Y7a} are independently selected from the group consisting of H, C₁₋₄ alkyl; or T^{Y1}, wherein C₁₋₄ alkyl is optionally substituted with one or more halogen, which are the same or different;

T^{Y1} is unsubstituted C₃₋₇ cycloalkyl; unsubstituted saturated 4 to 7 membered heterocyclyl; or saturated 7 to 11 membered heterobicyclyl;

X¹ is C(R^{6a}) or N; X² is C(R^{6b}) or N; X³ is CH, CF, COH or N; X⁴ is C(R^{6a}) or N; X⁵ is C(R^{6d}) or N, provided that at most two of X¹, X², X⁴, X⁵ are N;

R^{6a}, R^{6b}, R^{6c}, R^{6d} are independently selected from the group consisting of H; halogen; CN; C(O)OR⁷; OR⁷; C(O)R⁷; C(O)N(R⁷R^{7a}); S(O)₂N(R⁷R^{7a}); S(O)N(R⁷R^{7a}); S(O)₂R⁷; S(O)R⁷; N(R⁷)S(O)₂N(R^{7a}R^{7b}); N(R⁷)S(O)N(R^{7a}R^{7b}); SR⁷; N(R⁷R^{7a}); NO₂; OC(O)R⁷; N(R⁷)C(O)R^{7a}; N(R⁷)S(O)₂R^{7a}; N(R⁷)S(O)R^{7a}; N(R⁷)C(O)N(R^{7a}R^{7b}); N(R⁷)C(O)OR^{7a}; OC(O)N(R⁷R^{7a}); T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹¹, which are the same or different;

Optionally the pair R^{6a}/R^{6b} is joined to form a ring T³;

R⁷, R^{7a}, R^{7b} are independently selected from the group consisting of H; CN; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁸, which are the same or different;

R⁸ is halogen; CN; C(O)OR⁹; OR⁹; C(O)R⁹; C(O)N(R⁹R^{9a}); S(O)₂N(R⁹R^{9a}); S(O)N(R⁹R^{9a}); S(O)₂R⁹; S(O)R⁹; N(R⁹)S(O)₂N(R^{9a}R^{9b}); N(R⁹)S(O)N(R^{9a}R^{9b}); SR⁹; N(R⁹R^{9a}); NO₂; OC(O)R⁹; N(R⁹)C(O)R^{9a}; N(R⁹)S(O)₂R^{9a}; N(R⁹)S(O)R^{9a}; N(R⁹)C(O)N(R^{9a}R^{9b}); N(R⁹)C(O)OR^{9a}; OC(O)N(R⁹R^{9a}); or T²;

R⁹, R^{9a}, R^{9b} are independently selected from the group consisting of H; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹², which are the same or different;

R¹⁰ is halogen; CN; C(O)OR¹³; OR¹³; oxo (═O), where the ring is at least partially saturated; C(O)R¹³; C(O)N(R¹³R^{13a}); S(O)₂N(R¹³R^{13a}); S(O)N(R¹³R^{13a}); S(O)₂R¹³; S(O)R¹³; N(R¹³)S(O)₂N(R^{13a}R^{13b}); SR¹³; N(R¹³R^{13a}); NO₂; OC(O)R¹³; N(R¹³)C(O)R^{13a}; N(R¹³)S(O)₂R^{13a}; N(R¹³)S(O)R^{13a}; N(R¹³)C(O)N(R^{13a}R^{13b}); N(R¹³)C(O)OR^{13a}; OC(O)N(R¹³R^{13a}); C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R¹⁴, which are the same or different;

R¹³, R^{13a}; R^{13b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹⁴, which are the same or different;

R¹¹, R¹² are independently selected from the group consisting of halogen; CN; C(O)OR¹⁵; OR¹⁵; C(O)R¹⁵; C(O)N(R¹⁵R^{15a}); S(O)₂N(R¹⁵R^{15a}); S(O)N(R¹⁵R^{15a}); S(O)₂R¹⁵; S(O)R′5; N(R¹⁵)S(O)₂N(R^{15a}R^{15b}); N(R¹⁵)S(O)N(R^{15a}R^{15b}); SR¹⁵; N(R¹⁵R^{15a}); NO₂; OC(O)R¹⁵; N(R¹⁵)C(O)R^{15a}; N(R¹⁵)S(O)₂R^{15a}; N(R¹⁵)S(O)R^{15a}; N(R¹⁵)C(O)N(R^{15a}R^{15b}); N(R¹⁵)C(O)OR^{15a}; OC(O)N(R¹⁵R^{15a}); or T²;

R¹⁵, R^{15a}, R^{15b} are independently selected from the group consisting of H; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

R¹⁴ is halogen; CN; C(O)OR¹⁶; OR¹⁶; C(O)R¹⁶; C(O)N(R¹⁶R^{16a}); S(O)₂N(R¹⁶R^{16a}); S(O)N(R¹⁶R^{16a}); S(O)₂R¹⁶; S(O)R¹⁶; N(R¹⁶)S(O)₂N(R^{16a}R^{16b}); N(R¹⁶)S(O)N(R^{16a}R^{16b}); SR¹⁶; N(R¹⁶R^{16a}); NO₂; OC(O)R¹⁶; N(R¹⁶)C(O)R^{16a}; N(R¹⁶)S(O)₂R^{16a}; N(R¹⁶)S(O)R^{16a}; N(R¹⁶)C(O)N(R^{16a}R^{16b}); N(R¹⁶)C(O)OR^{16a}; or OC(O)N(R¹⁶R^{16a});

R¹⁶, R^{16a}, R^{16b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

T² is phenyl; naphthyl; indenyl; indanyl; C₃₋₇ cycloalkyl; 4 to 7 membered heterocyclyl; or 7 to 11 membered heterobicyclyl, wherein T² is optionally substituted with one or more R′⁷, which are the same or different;

T³ is phenyl; C₃₋₇ cycloalkyl; or 4 to 7 membered heterocyclyl; or 7 to 11 membered heterobicyclyl, wherein T³ is optionally substituted with one or more R¹⁸ which are the same or different;

R′⁷, R¹⁸ are independently selected from the group consisting of halogen; CN; C(O)OR¹⁹; OR¹⁹; oxo (═O), where the ring is at least partially saturated; C(O)R¹⁹; C(O)N(R¹⁹R^{19a}); S(O)₂N(R¹⁹R^{19a}); S(O)N(R¹⁹R^{19a}); S(O)₂R¹⁹; S(O)R¹⁹; N(R¹⁹)S(O)₂N(R^{19a}R^{19b}); N(R¹⁹)S(O)N(R^{19a}R¹⁹); SR¹⁹; N(R¹⁹R^{19a}); NO₂; OC(O)R¹⁹; N(R¹⁹)C(O)R^{19a}; N(R¹⁹)S(O)₂R^{19a}; N(R¹⁹)S(O)R^{19a}; N(R¹⁹)C(O)N(R^{19a}R^{19b}); N(R¹⁹) C(O)OR^{19a}; OC(O)N(R¹⁹R^{19a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R²⁰, which are the same or different;

R¹⁹, R^{19a}, R^{19b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R²⁰, which are the same or different;

R²⁰ is halogen; CN; C(O)OR²¹; OR²¹; C(O)R²¹; C(O)N(R²¹R^{21a}); S(O)₂N(R²¹R^{21a}); S(O)N(R²¹R^{21a}); S(O)₂R²¹; S(O)R²¹; N(R²¹)S(O)₂N(R^{21a}R^{21b}); N(R²¹)S(O)N(R^{21a}R^{21b}); SR²¹; N(R²¹R^{21a}); NO₂; OC(O)R²¹; N(R²¹)C(O)R^{21a}; N(R²¹)S(O)₂R^{21a}; N(R²¹)S(O)R^{21a}; N(R²¹)C(O)N(R^{21a}R^{21b}); N(R²¹)C(O)OR^{21a}; or OC(O)N(R²¹R^{21a});

R²¹, R^{21a}, R^{21b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different, provided that the following compounds are excluded:

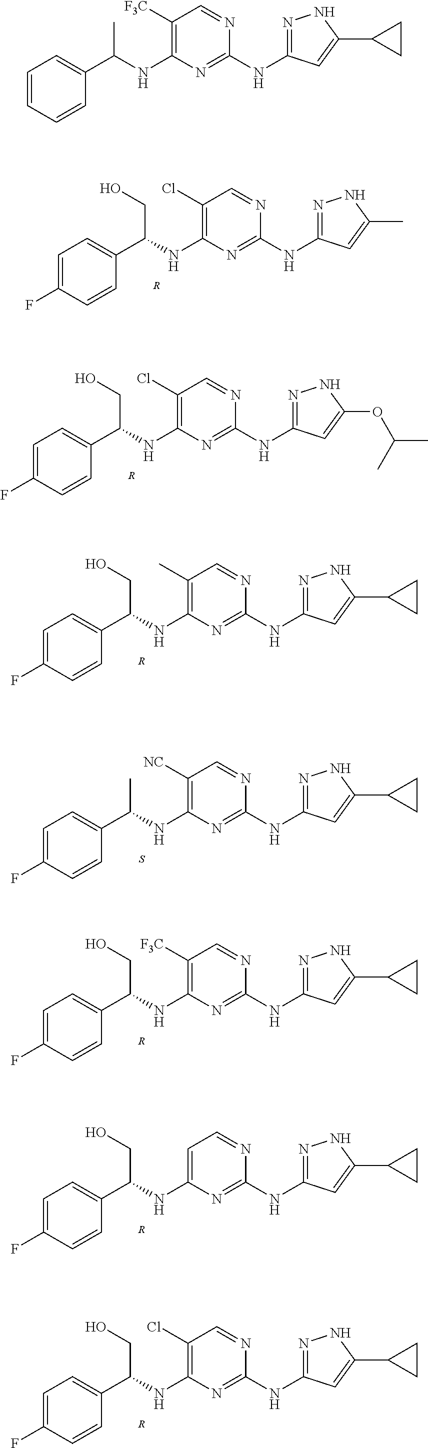

The compounds disclaimed from the scope of formula (I) are shown in WO 2006/117560 A as examples on pages 27 to 29.

In case a variable or substituent can be selected from a group of different variants and such variable or substituent occurs more than once the respective variants can be the same or different.

Within the meaning of the present invention the terms are used as follows:

The term “optionally substituted” means unsubstituted or substituted. Generally—but not limited to-, “one or more substituents” means one, two or three, preferably one or two and more preferably one. Generally these substituents can be the same or different.

“Alkyl” means a straight-chain or branched hydrocarbon chain. Each hydrogen of an alkyl carbon may be replaced by a substituent as further specified herein.

“Alkenyl” means a straight-chain or branched hydrocarbon chain that contains at least one carbon-carbon double bond. Each hydrogen of an alkenyl carbon may be replaced by a substituent as further specified herein.

“Alkynyl” means a straight-chain or branched hydrocarbon chain that contains at least one carbon-carbon triple bond. Each hydrogen of an alkynyl carbon may be replaced by a substituent as further specified herein.

“C₁₋₄ alkyl” means an alkyl chain having 1-4 carbon atoms, e.g. if present at the end of a molecule: methyl, ethyl, n-propyl, isopropyl, n-butyl, isobutyl, sec-butyl, tert-butyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group. Each hydrogen of a C₁₋₄ alkyl carbon may be replaced by a substituent as further specified herein. “C₂₋₄ alkyl” is defined accordingly.

“C₁₋₆ alkyl” means an alkyl chain having 1-6 carbon atoms, e.g. if present at the end of a molecule: C₁₋₄ alkyl, methyl, ethyl, n-propyl, isopropyl, n-butyl, isobutyl, sec-butyl; tert-butyl, n-pentyl, n-hexyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group. Each hydrogen of a C₁₋₆ alkyl carbon may be replaced by a substituent as further specified herein.

“C₂₋₆ alkenyl” means an alkenyl chain having 2 to 6 carbon atoms, e.g. if present at the end of a molecule: —CH═CH₂, —CH═CH—CH₃, —CH₂—CH═CH₂, —CH═CH—CH₂—CH₃, —CH═CH—CH═CH₂, or e.g. —CH═CH—, when two moieties of a molecule are linked by the alkenyl group. Each hydrogen of a C₂₋₆ alkenyl carbon may be replaced by a substituent as further specified herein.

“C₂₋₆ alkynyl” means an alkynyl chain having 2 to 6 carbon atoms, e.g. if present at the end of a molecule: —C≡CH, —CH₂—C≡CH, CH₂—CH₂—C≡CH, CH₂—C≡C—CH₃, or e.g. —C≡C— when two moieties of a molecule are linked by the alkynyl group. Each hydrogen of a C₂₋₆ alkynyl carbon may be replaced by a substituent as further specified herein.

“C₃₋₇ cycloalkyl” or “C₃₋₇ cycloalkyl ring” means a cyclic alkyl chain having 3-7 carbon atoms, e.g. cyclopropyl, cyclobutyl, cyclopentyl, cyclohexyl, cyclohexenyl, cycloheptyl. Preferably, cyloalkyl refers to cyclopropyl, cyclobutyl, cyclopentyl, cyclohexyl, or cycloheptyl. Each hydrogen of a cycloalkyl carbon may be replaced by a substituent as further specified herein. The term “C₃₋₅ cycloalkyl” or “C₃₋₅ cycloalkyl ring” is defined accordingly.

“Halogen” means fluoro, chloro, bromo or iodo. It is generally preferred that halogen is fluoro or chloro.

“4 to 7 membered heterocyclyl” or “4 to 7 membered heterocycle” means a ring with 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 4 to 7 membered heterocycles are azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine or homopiperazine. The term “5 to 6 membered heterocyclyl” or “5 to 6 membered heterocycle” is defined accordingly.

“Saturated 4 to 7 membered heterocyclyl” or “saturated 4 to 7 membered heterocycle” means fully saturated “4 to 7 membered heterocyclyl” or “4 to 7 membered heterocycle”.

“5 membered aromatic heterocyclyl” or “5 membered aromatic heterocycle” means a heterocycle derived from cyclopentadienyl, where at least one carbon atom is replaced by a heteoatom selected from the group consisting of sulfur (including —S(O)—, —S(O)₂—), oxygen and nitrogen (including ═N(O)—). Examples for such heterocycles are furan, thiophene, pyrrole, imidazole, pyrazole, oxazole, isoxazole, thiazole, isothiazole, thiadiazole, triazole, tetrazole.

“7 to 11 membered heterobicyclyl” or “7 to 11 membered heterobicycle” means a heterocyclic system of two rings with 7 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 7 to 11 membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquino line, de cahydroisoquinoline, tetrahydroisoquinoline, dihydroisoquinoline, benzazepine, purine or pteridine. The term 7 to 11 membered heterobicycle also includes spiro structures of two rings like 1,4-dioxa-8-azaspiro[4.5]decane or 2-oxa-6-azaspiro[3.3]heptan-6-yl or bridged heterocycles like 8-aza-bicyclo[3.2.1]octane or 2,5-diazabicyclo[2.2.2]octan-2-yl.

“Saturated 7 to 11 membered heterobicyclyl” or “saturated 7 to 11 membered heterobicycle” means fully saturated “7 to 11 membered heterobicyclyl” or “7 to 11 membered heterobicycle”.

Preferred compounds of formula (I) are those compounds in which one or more of the residues contained therein have the meanings given below, with all combinations of preferred substituent definitions being a subject of the present invention. With respect to all preferred compounds of the formula (I) the present invention also includes all tautomeric and stereoisomeric forms and mixtures thereof in all ratios, and their pharmaceutically acceptable salts.

In preferred embodiments of the present invention, the substituents mentioned below independently have the following meaning Hence, one or more of these substituents can have the preferred or more preferred meanings given below.

Preferably, n is 0.

Preferably, in formula (I) R^{Y1}, R^{Y2} and Y⁰ are defined to give formula (Ia) (Ib), (Ic), or (Id):

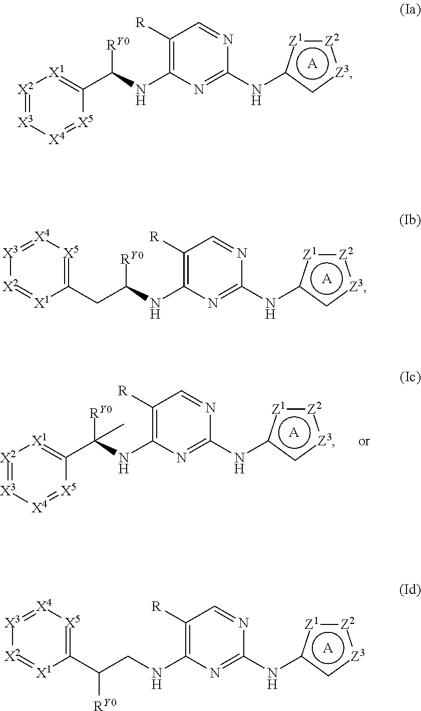

Preferably, Z² is N(R¹) in formulae (Ia) to (Id) with R¹ being other than H. Even more preferred is formula (Ia).

Preferably, R^{Y0} is unsubstituted C₂₋₄ alkyl; CH₂CH₂OR^{Y5}; CH₂CH₂C(O)T^{Y1}; CH₂CH₂C(O)OR^{Y5}; CH₂CH₂OC(O)R^{Y5}; CH₂CH₂N(R^{Y5}R^{Y5a}); CH₂CH₂N(R^{Y5})C(O)R^{Y5a}; CH₂CH₂C(O)N(R^{Y5}R^{Y5a}); CH₂OR^{Y5}; CH₂C(O)T^{Y1}; CH₂C(O)OR^{Y5}; CH₂OC(O)R^{Y5}; CH₂N(R^{Y5}R^{Y5a}); CH₂N(R^{Y5})C(O)R^{Y5a}; CH₂C(O)N(R^{Y5}R^{Y5a}); C(O)T^{Y1}; C(O)OR^{Y5}; or C(O)N(R^{Y5}R^{Y5a}). More preferably, R^{Y0} is CH₂CH₃; CH₂OR^{Y5}; C(O)OR^{Y5}; C(O)N(R^{Y5}R^{Y5a}); or C(O)T^{Y1}. Even more preferably, R^{Y0} is CH₂OR^{Y5}, especially CH₂OH. Even more preferably, R^{Y0} is CH₂OH; CH₂CH₃; C(O)OH; C(O)OCH₃; C(O)NHCH₃; C(O)N(CH₃)₂; C(O)NHCH₂CH₃; C(O)NHCH₂CH₂CH₂OCH₃; pyrrolidin-1-ylcarbonyl; or piperidin-1-ylcarbonyl. Even more preferably, R^{Y0} is CH₂OCH₃; cyclopentylaminocarbonyl; CH₂CH₂OH; or 2,2,2-trifluorethylaminocarbonyl.

Preferably, ring A is a pyrrolyl or pyrazolyl ring; more preferably a pyrazolyl ring. Even more preferred a ring selected from the group consisting of:

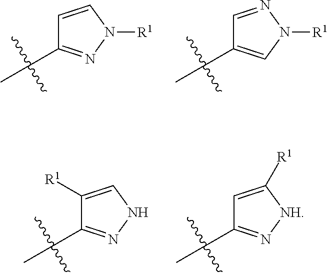

Even more preferred is

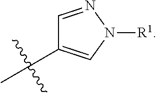

Preferably, ring A is unsubstituted. More preferably, ring A is substituted with one or two (preferably one) R¹, which are different from H and the same or different.

Preferably, one of Z¹, Z², Z³ is N(R¹) and R¹ is different from H.

Preferably, R¹ is C(O)OR²; C(O)R²; or C(O)N(R²R^{2a}) (preferably C(O)NHR²). Preferably, R¹ is morpholin-4-ylcarbonyl. Preferably, R¹ is N-methylpyrrolidin-2-on-3-yl.

Preferably, R¹ is unsubstituted C₁₋₄ alkyl (preferably, methyl); or C₁₋₄ alkyl, substituted with one or two (preferably one) R³, which are the same or different.

Preferably, R³ is halogen; OR⁴; C(O)OR⁴; C(O)T′; or C(O)N(R⁴R^{4a}). Also preferably, R³ is OR⁴; C(O)OR⁴; or C(O)N(R⁴R^{4a}). More preferably, R³ is NH₂, or halogen. More preferably, R³ is C(O)N(R⁴R^{4a}). Even more preferably R³ is OH; C(O)OC₁₋₄ alkyl (preferably ethyl or 2-propyl); C(O)NHC₁₋₄ alkyl (preferably methyl); or C(O)N(C₁₋₄ alkyl)₂ (preferably dimethyl). Even more preferably R³ is C(O)NH₂.

More preferably, R¹ is selected from the group CH₂CH₂OH; CH₂CH(OH)CH₃; CH₂C(O)OH; CH₂C(O)OC₁₋₄ alkyl (preferably ethyl or 2-propyl); CH₂C(O)NHC₁₋₄ alkyl (preferably methyl); or CH₂C(O)N(C₁₋₄ alkyl)₂ (preferably dimethyl). Even more preferably, R¹ is CH₂C(CH₃)₂OH; (CH₂)₃OH; cyclopropylaminocarbonylmethyl; CH₂C(O)N(CH₃)CH₂CN; C(CH₃)₂C(O)NH₂; CH₂C(O)NH(CH₂)₂N(CH₃)₂; CH₂C(O)NH(CH₂)₃N(CH₃)₂; morpholin-4-ylcarbonylmethyl; 3-aminopropyl; isopropyloxyethyl; CH₂C(O)NHCH(CH₃)₂; CH₂C(O)NHCH(CH₃)CH₂OH; or 2,2-difluoroethyl. Even more preferably, R¹ is CH₃ or CH₂CH₂OH. Even more preferably, R¹ is CH₂C(O)NH₂ or CH₂C(O)NHCH₃.

Preferably, R is F; Cl; CF₃; or CH₃. More preferably, R is Cl.

Preferably, none of X¹, X², X⁴, X⁵ is N. Preferably, X³ is CH. More preferably X¹, X², X⁴, X⁵ are CH.

Preferably, at most three (preferably at most two, even more preferably at most one) of R^{6a}, R^{6b}, R^{6c}, R^{6d} are other than H. Accordingly, in a preferred embodiment none of R^{6a}R^{6b}, R^{6c}, R^{6d} is other than H and in another preferred embodiment one of R^{6a}, R^{6b}, R^{6c}, R^{6d} is other than H.

Preferably, R^{6a}, R^{6b}, R^{6c}, R^{6d} are independently selected from the group consisting of H; halogen; CN; C(O)OR⁷; OR⁷; C(O)R⁷; C(O)N(R⁷R^{7a}); S(O)₂N(R⁷R^{7a}); S(O)N(R⁷R^{7a}); S(O)₂R⁷; S(O)R⁷; SR⁷; N(R⁷R^{7a}); NO₂; OC(O)R⁷; N(R⁷)C(O)R^{7a}; N(R⁷)C(O)N(R^{7a}R^{7b}); N(R⁷)C(O)OR^{7a}; OC(O)N(R⁷R^{7a}); T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹¹, which are the same or different.

Preferably, R^{6a}, R^{6b}, R^{6c}, R^{6d} are independently selected from the group consisting of H; halogen (preferably F); or T², like 2-oxa-6-azaspiro[3.3]heptan-6-yl, N-methylpyrazol-4-yl.

Preferably, R^{6a}, R^{6b}, R^{6c}, R^{6d} are independently selected from the group consisting of H; halogen; CF₃; OR⁷, like OCH₃; or T², like N-methylpyrazol-4-yl or morpholin-3-on-4-yl. More preferably, R^{6a}, R^{6b}, R^{6c}, R^{6d} are independently selected from the group consisting of H; and halogen (preferably F).

Compounds of formula (I) in which some or all of the above-mentioned groups have the preferred meanings are also an object of the present invention.

Further preferred compounds of the present invention are selected from the group consisting of
2-((5-chloro-2-((1-methyl-1H-pyrazol-4-yl)amino)pyrimidin-4-yl)amino)-2-phenylethanol;
2-((5-fluoro-2-((1-methyl-1H-pyrazol-4-yl)amino)pyrimidin-4-yl)amino)-2-phenylethanol;
(S)-2-((5-fluoro-2-((1-methyl-1H-pyrazol-4-yl)amino)pyrimidin-4-yl)amino)-2-phenylethanol;
(S)-2-((5-chloro-2-((1-methyl-1H-pyrazol-4-yl)amino)pyrimidin-4-yl)amino)-2-phenylethanol;
(R)-5-chloro-N2-(1-methyl-1H-pyrazol-4-yl)-N4-(1-phenylpropyl)pyrimidine-2,4-diamine;
(S)-2-((2-((1-methyl-1H-pyrazol-4-yl)amino)-5-(trifluoromethyl)pyrimidin-4-yl)amino)-2-phenylethanol;
2-((5-chloro-2-((1-methyl-1H-pyrazol-4-yl)amino)pyrimidin-4-yl)amino)-2-phenylpropan-1-ol;
2-((2-((1-methyl-1H-pyrazol-4-yl)amino)-5-(trifluoromethyl)pyrimidin-4-yl)amino)-2-phenylpropan-1-ol;
(S)-2-((5-chloro-2-((1-(2-hydroxyethyl)-1H-pyrazol-4-yl)amino)pyrimidin-4-yl)amino)-2-phenylethanol;
(S)-2-((5-fluoro-2-((1-(2-hydroxyethyl)-1H-pyrazol-4-yl)amino)pyrimidin-4-yl)amino)-2-phenylethanol;
(S)-2-((5-chloro-2-((1-methyl-1H-pyrazol-3-yl)amino)pyrimidin-4-yl)amino)-2-phenylethanol;
2-((2-((1-(2-hydroxyethyl)-1H-pyrazol-4-yl)amino)-5-methylpyrimidin-4-yl)amino)-2-phenylethanol;
(S)-(4-((5-chloro-4-((2-hydroxy-1-phenylethyl)amino)pyrimidin-2-yl)amino)-1H-pyrrol-2-yl)(morpholino)methanone;
2-((5-chloro-2-((1-methyl-1H-pyrazol-4-yl)amino)pyrimidin-4-yl)amino)-3-phenylpropan-1-ol;
methyl 2-((5-chloro-2-((1-(2-hydroxyethyl)-1H-pyrazol-4-yl)amino)pyrimidin-4-yl)amino)-2-phenylacetate;
methyl 2-((5-chloro-2-((1-methyl-1H-pyrazol-4-yl)amino)pyrimidin-4-yl)amino)-2-phenylacetate;
2-((5-chloro-2-((1-(2-hydroxyethyl)-1H-pyrazol-4-yl)amino)pyrimidin-4-yl)amino)-2-phenylacetic acid;
2-((5-chloro-2-((1-methyl-1H-pyrazol-4-yl)amino)pyrimidin-4-yl)amino)-2-phenylacetic acid;
2-((5-chloro-2-((1-(2-hydroxyethyl)-1H-pyrazol-4-yl)amino)pyrimidin-4-yl)amino)-3-phenylpropan-1-ol;
(S)-2-((5-chloro-2-((1-methyl-1H-pyrazol-4-yl)amino)pyrimidin-4-yl)amino)-N-methyl-2-phenylacetamide;
(S)-isopropyl 2-(4-((5-chloro-4-((2-hydroxy-1-phenylethyl)amino)pyrimidin-2-yl)amino)-1H-pyrazol-1-yl)acetate;
(S)-ethyl 2-(4-((5-chloro-4-((2-hydroxy-1-phenylethyl)amino)pyrimidin-2-yl)amino)-1H-pyrazol-1-yl)acetate;
(S)-2-(4-((5-chloro-4-((2-hydroxy-1-phenylethyl)amino)pyrimidin-2-yl)amino)-1H-pyrazol-1-yl)-N-methylacetamide;
(S)-2-(4-((5-chloro-4-((2-hydroxy-1-phenylethyl)amino)pyrimidin-2-yl)amino)-1H-pyrazol-1-yl)acetic acid;
(S)-2-((5-chloro-2-((1-methyl-1H-pyrazol-4-yl)amino)pyrimidin-4-yl)amino)-N,N-dimethyl-2-phenylacetamide;
(S)-2-((5-chloro-2-((1-(2-hydroxyethyl)-1H-pyrazol-4-yl)amino)pyrimidin-4-yl)amino)-N,N-dimethyl-2-phenylacetamide;
(S)-2-((5-chloro-2-((1-(2-hydroxyethyl)-1H-pyrazol-4-yl)amino)pyrimidin-4-yl)amino)-N-ethyl-2-phenylacetamide;
(S)-2-((5-chloro-2-((1-methyl-1H-pyrazol-4-yl)amino)pyrimidin-4-yl)amino)-N-ethyl-2-phenylacetamide;
(S)-2-((5-chloro-2-((1-methyl-1H-pyrazol-4-yl)amino)pyrimidin-4-yl)amino)-N-(2-methoxyethyl)-2-phenylacetamide;
(S)-2-(4-((5-chloro-4-((2-hydroxy-1-phenylethyl)amino)pyrimidin-2-yl)amino)-1H-pyrazol-1-yl)-N,N-dimethylacetamide;

2-(4-((5-chloro-4-((1-(2,6-difluorophenyl)-2-hydroxyethyl)amino)pyrimidin-2-yl)amino)-1H-pyrazol-1-yl)-N-methylacetamide;
2-(4-((5-chloro-4-((1-(3-fluorophenyl)-2-hydroxyethyl)amino)pyrimidin-2-yl)amino)-1H-pyrazol-1-yl)-N-methylacetamide;
2-(4-((5-chloro-4-((1-(2-fluorophenyl)-2-hydroxyethyl)amino)pyrimidin-2-yl)amino)-1H-pyrazol-1-yl)-N-methylacetamide;
(S)-2-((5-chloro-2-((1-methyl-1H-pyrazol-4-yl)amino)pyrimidin-4-yl)amino)-2-phenyl-1-(pyrrolidin-1-yl)ethanone;
(S)-2-((5-chloro-2-((1-(2-hydroxyethyl)-1H-pyrazol-4-yl)amino)pyrimidin-4-yl)amino)-2-phenyl-1-(pyrrolidin-1-yl)ethanone;
2-((5-chloro-2-((1-(2-hydroxyethyl)-1H-pyrazol-4-yl)amino)pyrimidin-4-yl)amino)-2-(2-fluorophenyl)ethanol;
   2-((5-chloro-2-((1-methyl-1H-pyrazol-4-yl)amino)pyrimidin-4-yl)amino)-2-(2-fluorophenyl)ethanol;
1-(4-((5-chloro-4-((1-(3-fluorophenyl)-2-hydroxyethyl)amino)pyrimidin-2-yl)amino)-1H-pyrazol-1-yl)propan-2-ol;
2-((5-chloro-2-((1-(2-hydroxyethyl)-1H-pyrazol-4-yl)amino)pyrimidin-4-yl)amino)-2-(3-fluorophenyl)ethanol;
2-((5-chloro-2-((1-methyl-1H-pyrazol-4-yl)amino)pyrimidin-4-yl)amino)-2-(3-fluorophenyl)ethanol;
2-((5-chloro-2-((1-(2-hydroxyethyl)-1H-pyrazol-3-yl)amino)pyrimidin-4-yl)amino)-2-(3-fluorophenyl)ethanol;
1-(4-((5-chloro-4-((1-(2,6-difluorophenyl)-2-hydroxyethyl)amino)pyrimidin-2-yl)amino)-1H-pyrazol-1-yl)propan-2-ol;
2-((5-chloro-2-((1-(2-hydroxyethyl)-1H-pyrazol-4-yl)amino)pyrimidin-4-yl)amino)-2-(2,6-difluorophenyl)ethanol;
2-((5-chloro-2-((1-methyl-1H-pyrazol-4-yl)amino)pyrimidin-4-yl)amino)-2-(2,6-difluorophenyl)ethanol;
2-((5-chloro-2-((1-(2-hydroxyethyl)-1H-pyrazol-3-yl)amino)pyrimidin-4-yl)amino)-2-(2,6-difluorophenyl)ethanol;
(S)-2-((5-chloro-2-((1-methyl-1H-pyrazol-4-yl)amino)pyrimidin-4-yl)amino)-N-(2-hydroxyethyl)-2-phenylacetamide;
(S)-2-((5-chloro-2-((1-(2-hydroxyethyl)-1H-pyrazol-4-yl)amino)pyrimidin-4-yl)amino)-N-(2-hydroxyethyl)-2-phenylacetamide;
(S)-2-((5-chloro-2-((1-(2-hydroxyethyl)-1H-pyrazol-4-yl)amino)pyrimidin-4-yl)amino)-N-(2-methoxyethyl)-2-phenylacetamide;
(S)-2-((5-chloro-2-((1-methyl-1H-pyrazol-4-yl)amino)pyrimidin-4-yl)amino)-2-phenyl-1-(piperidin-1-yl)ethanone;
2-((5-chloro-2-((1-(2-hydroxyethyl)-1H-pyrazol-3-yl)amino)pyrimidin-4-yl)amino)-2-(2-fluorophenyl)ethanol;
(S)-2-((5-chloro-2-((1-(2-hydroxyethyl)-1H-pyrazol-4-yl)amino)pyrimidin-4-yl)amino)-2-phenyl-1-(piperidin-1-yl)ethanone; and
2-(3-(2-oxa-6-azaspiro[3.3]heptan-6-yl)phenyl)-2-((5-chloro-2-((1-methyl-1H-pyrazol-4-yl)amino)pyrimidin-4-yl)amino)ethanol.

Further preferred compounds of the present invention are selected from the group consisting of
(S)-5-chloro-N4-(2-methoxy-1-phenylethyl)-N2-(1-methyl-1H-pyrazol-4-yl)pyrimidine-2,4-diamine;
(S)-2-(4-((5-chloro-4-((2-hydroxy-1-phenylethyl)amino)pyrimidin-2-yl)amino)-1H-pyrazol-1-yl)acetamide;
1-(4-((5-chloro-4-((1-(2-fluorophenyl)-2-hydroxyethyl)amino)pyrimidin-2-yl)amino)-1H-pyrazol-1-yl)-2-methylpropan-2-ol;
(S)-2-((5-chloro-2-((1-methyl-1H-pyrazol-4-yl)amino)pyrimidin-4-yl)amino)-2-(2-fluorophenyl)ethanol;
(S)-2-((5-chloro-2-((1-methyl-1H-pyrazol-4-yl)amino)pyrimidin-4-yl)amino)-N-cyclopentyl-2-phenylacetamide;
(S)-2-((5-chloro-2-((1-(2-hydroxyethyl)-1H-pyrazol-4-yl)amino)pyrimidin-4-yl)amino)-N-cyclopentyl-2-phenylacetamide;
1-(4-((5-chloro-4-((1-(3-fluorophenyl)-2-hydroxyethyl)amino)pyrimidin-2-yl)amino)-1H-pyrazol-1-yl)-2-methylpropan-2-ol;
1-(4-((5-chloro-4-((1-(2,6-difluorophenyl)-2-hydroxyethyl)amino)pyrimidin-2-yl)amino)-1H-pyrazol-1-yl)-2-methylpropan-2-ol;
3-(4-((5-chloro-4-((1-(2-fluorophenyl)-2-hydroxyethyl)amino)pyrimidin-2-yl)amino)-1H-pyrazol-1-yl)propan-1-ol;
(R)-3-((5-chloro-2-((1-(2-hydroxyethyl)-1H-pyrazol-4-yl)amino)pyrimidin-4-yl)amino)-3-phenylpropan-1-ol;
(R)-2-(4-((5-chloro-4-((3-hydroxy-1-phenylpropyl)amino)pyrimidin-2-yl)amino)-1H-pyrazol-1-yl)-N-methylacetamide;
(S)-2-(4-((5-chloro-4-((2-hydroxy-1-phenylethyl)amino)pyrimidin-2-yl)amino)-1H-pyrazol-1-yl)-N-isopropylacetamide;
(S)-2-(4-((5-chloro-4-((2-hydroxy-1-phenylethyl)amino)pyrimidin-2-yl)amino)-1H-pyrazol-1-yl)-N-cyclopropylacetamide;
2-((5-chloro-2-((1-methyl-1H-pyrazol-4-yl)amino)pyrimidin-4-yl)amino)-2-(3-(1-methyl-1H-pyrazol-4-yl)phenyl)ethanol;
(S)-2-(4-((5-chloro-4-((2-hydroxy-1-phenylethyl)amino)pyrimidin-2-yl)amino)-1H-pyrazol-1-yl)-N-(cyanomethyl)-N-methylacetamide;
2-((5-chloro-2-((1-methyl-1H-pyrazol-4-yl)amino)pyrimidin-4-yl)amino)-2-(4-fluorophenyl)ethanol;
2-((5-chloro-2-((1-(2-hydroxyethyl)-1H-pyrazol-4-yl)amino)pyrimidin-4-yl)amino)-2-(4-fluorophenyl)ethanol;
(S)-2-(4-((5-chloro-4-((1-(2-fluorophenyl)-2-hydroxyethyl)amino)pyrimidin-2-yl)amino)-1H-pyrazol-1-yl)-2-methylpropanamide;
(S)-2-((5-chloro-2-((1-methyl-1H-pyrazol-4-yl)amino)pyrimidin-4-yl)amino)-2-(2-methoxyphenyl)ethanol;
(S)-2-((2-((1H-pyrazol-4-yl)amino)-5-chloropyrimidin-4-yl)amino)-2-phenylethanol;
(S)-2-((5-chloro-2-((1-(2-hydroxyethyl)-1H-pyrazol-4-yl)amino)pyrimidin-4-yl)amino)-2-(2-methoxyphenyl)ethanol;
(S)-2-((5-chloro-2-((1-(2-hydroxyethyl)-1H-pyrazol-4-yl)amino)pyrimidin-4-yl)amino)-2-(2-fluorophenyl)ethanol;
(S)-2-(4-((5-chloro-4-((1-(2-fluorophenyl)-2-hydroxyethyl)amino)pyrimidin-2-yl)amino)-1H-pyrazol-1-yl)acetamide;
(S)-2-((2-((1H-pyrazol-4-yl)amino)-5-chloropyrimidin-4-yl)amino)-2-(2-fluorophenyl)ethanol;
(S)-2-((5-chloro-2-((1-(2-hydroxyethyl)-1H-pyrazol-4-yl)amino)pyrimidin-4-yl)amino)-2-(pyridin-2-yl)ethanol;
(S)-2-(4-((5-chloro-4-((2-hydroxy-1-(pyridin-2-yl)ethyl)amino)pyrimidin-2-yl)amino)-1H-pyrazol-1-yl)acetamide;
(S)-2-((5-chloro-2-((1-methyl-1H-pyrazol-4-yl)amino)pyrimidin-4-yl)amino)-2-(3-methoxyphenyl)ethanol;
(S)-2-((5-chloro-2-((1-(2-hydroxyethyl)-1H-pyrazol-4-yl)amino)pyrimidin-4-yl)amino)-2-(3-methoxyphenyl)ethanol;
(S)-2-(4-((5-chloro-4-((2-hydroxy-1-(3-methoxyphenyl)ethyl)amino)pyrimidin-2-yl)amino)-1H-pyrazol-1-yl)acetamide;
(S)-2-((5-chloro-2-((1-methyl-1H-pyrazol-4-yl)amino)pyrimidin-4-yl)amino)-2-(3-fluorophenyl)ethanol;
(S)-2-((5-chloro-2-((1-(2-hydroxyethyl)-1H-pyrazol-4-yl)amino)pyrimidin-4-yl)amino)-2-(3-fluorophenyl)ethanol;
(S)-2-(4-((5-chloro-4-((1-(3-fluorophenyl)-2-hydroxyethyl)amino)pyrimidin-2-yl)amino)-1H-pyrazol-1-yl)acetamide;
(S)-2-((2-((1H-pyrazol-4-yl)amino)-5-chloropyrimidin-4-yl)amino)-2-(3-fluorophenyl)ethanol;
(S)-2-(4-((5-chloro-4-((2-hydroxy-1-phenylethyl)amino)pyrimidin-2-yl)amino)-1H-pyrazol-1-yl)-N-(2-(dimethylamino)ethyl)acetamide;
(S)-2-(4-((5-chloro-4-((2-hydroxy-1-phenylethyl)amino)pyrimidin-2-yl)amino)-1H-pyrazol-1-yl)-N-(3-(dimethylamino)propyl)acetamide;
(S)-2-((5-chloro-2-((1-methyl-1H-pyrazol-4-yl)amino)pyrimidin-4-yl)amino)-2-(pyridin-3-yl)ethanol;
(S)-2-((5-chloro-2-((1-(2-hydroxyethyl)-1H-pyrazol-4-yl)amino)pyrimidin-4-yl)amino)-2-(pyridin-3-yl)ethanol;
2-(3-bromophenyl)-2-((5-chloro-2-((1-(2-hydroxyethyl)-1H-pyrazol-4-yl)amino)pyrimidin-4-yl)amino)ethanol;
2-((5-chloro-2-((1-(2-hydroxyethyl)-1H-pyrazol-4-yl)amino)pyrimidin-4-yl)amino)-2-(3-(1-methyl-1H-pyrazol-4-yl)phenyl)ethanol;
(S)-2-((5-chloro-2-((1-ethyl-1H-pyrazol-4-yl)amino)pyrimidin-4-yl)amino)-2-(2-methoxyphenyl)ethanol;
(S)-2-((5-chloro-2-((1-ethyl-1H-pyrazol-4-yl)amino)pyrimidin-4-yl)amino)-2-(3-methoxyphenyl)ethanol;
(S)-2-((5-chloro-2-((1-ethyl-1H-pyrazol-4-yl)amino)pyrimidin-4-yl)amino)-2-(pyridin-3-yl)ethanol;
(S)-2-(4-((5-chloro-4-((2-hydroxy-1-(2-methoxyphenyl)ethyl)amino)pyrimidin-2-yl)amino)-1H-pyrazol-1-yl)-N-methylacetamide;
(S)-2-(4-((5-chloro-4-((2-hydroxy-1-(3-methoxyphenyl)ethyl)amino)pyrimidin-2-yl)amino)-1H-pyrazol-1-yl)-N-methylacetamide;
(S)-2-(4-((5-chloro-4-((1-(2-fluorophenyl)-2-hydroxyethyl)amino)pyrimidin-2-yl)amino)-1H-pyrazol-1-yl)-N-methylacetamide;
(S)-2-(4-((5-chloro-4-((2-hydroxy-1-(2-methoxyphenyl)ethyl)amino)pyrimidin-2-yl)amino)-1H-pyrazol-1-yl)-1-morpholinoethanone;
(S)-2-(4-((5-chloro-4-((2-hydroxy-1-(3-methoxyphenyl)ethyl)amino)pyrimidin-2-yl)amino)-1H-pyrazol-1-yl)-1-morpholinoethanone;
(S)-2-(4-((5-chloro-4-((1-(2-fluorophenyl)-2-hydroxyethyl)amino)pyrimidin-2-yl)amino)-1H-pyrazol-1-yl)-1-morpholinoethanone;
(S)-2-(4-((5-chloro-4-((2-hydroxy-1-(pyridin-3-yl)ethyl)amino)pyrimidin-2-yl)amino)-1H-pyrazol-1-yl)-1-morpholinoethanone;
(S)-2-((5-chloro-2-((1-isopropyl-1H-pyrazol-4-yl)amino)pyrimidin-4-yl)amino)-2-(2-methoxyphenyl)ethanol;
(S)-2-((5-chloro-2-((1-isopropyl-1H-pyrazol-4-yl)amino)pyrimidin-4-yl)amino)-2-(3-methoxyphenyl)ethanol;
(S)-2-((5-chloro-2-((1-isopropyl-1H-pyrazol-4-yl)amino)pyrimidin-4-yl)amino)-2-(pyridin-3-yl)ethanol;
(S)-2-(4-((5-fluoro-4-((2-hydroxy-1-phenylethyl)amino)pyrimidin-2-yl)amino)-1H-pyrazol-1-yl)acetamide;
(S)-2-((5-chloro-2-((1-ethyl-1H-pyrazol-4-yl)amino)pyrimidin-4-yl)amino)-2-(2-fluorophenyl)ethanol;
(S)-2-((5-chloro-2-((1-isopropyl-1H-pyrazol-4-yl)amino)pyrimidin-4-yl)amino)-2-(2-fluorophenyl)ethanol;
(S)-2-(4-((5-fluoro-4-((2-hydroxy-1-phenylethyl)amino)pyrimidin-2-yl)amino)-1H-pyrazol-1-yl)-N-methylacetamide;
(S)-2-(4-((5-fluoro-4-((2-hydroxy-1-phenylethyl)amino)pyrimidin-2-yl)amino)-1H-pyrazol-1-yl)-1-morpholinoethanone;
(S)-2-((2-((1-(3-aminopropyl)-1H-pyrazol-4-yl)amino)-5-chloropyrimidin-4-yl)amino)-2-phenylethanol;
(S)-2-((5-chloro-2-((1-(2-isopropoxyethyl)-1H-pyrazol-4-yl)amino)pyrimidin-4-yl)amino)-2-phenylethanol;
(S)-2-(4-((5-chloro-4-((1-(2-fluorophenyl)-2-hydroxyethyl)amino)pyrimidin-2-yl)amino)-1H-pyrazol-1-yl)-N-isopropylacetamide;
2-(4-((5-chloro-4-(((S)-1-(2-fluorophenyl)-2-hydroxyethyl)amino)pyrimidin-2-yl)amino)-1H-pyrazol-1-yl)-N-((S)-1-hydroxypropan-2-yl)acetamide;
2-((5-chloro-2-((1-methyl-1H-pyrazol-4-yl)amino)pyrimidin-4-yl)amino)-2-(2-chlorophenyl)ethanol;
2-(4-((5-chloro-4-((1-(2-chlorophenyl)-2-hydroxyethyl)amino)pyrimidin-2-yl)amino)-1H-pyrazol-1-yl)acetamide;
2-(4-((5-chloro-4-((1-(2-chlorophenyl)-2-hydroxyethyl)amino)pyrimidin-2-yl)amino)-1H-pyrazol-1-yl)-N-methylacetamide;
2-((5-chloro-2-((1-(2-hydroxyethyl)-1H-pyrazol-4-yl)amino)pyrimidin-4-yl)amino)-2-(2-chlorophenyl)ethanol;
2-((5-chloro-2-((1-methyl-1H-pyrazol-4-yl)amino)pyrimidin-4-yl)amino)-2-(3,5-difluorophenyl)ethanol;
2-((5-chloro-2-((1-(2-hydroxyethyl)-1H-pyrazol-4-yl)amino)pyrimidin-4-yl)amino)-2-(3,5-difluorophenyl)ethanol;
2-(4-((5-chloro-4-((1-(3,5-difluorophenyl)-2-hydroxyethyl)amino)pyrimidin-2-yl)amino)-1H-pyrazol-1-yl)-N-methylacetamide;
4-(3-(1-((5-chloro-2-((1-methyl-1H-pyrazol-4-yl)amino)pyrimidin-4-yl)amino)-2-hydroxyethyl)phenyl)morpholin-3-one;
5-chloro-N4-(1-(2-fluorophenyl)propyl)-N2-(1-methyl-1H-pyrazol-4-yl)pyrimidine-2,4-diamine;
2-(4-((5-chloro-4-((1-(2-fluorophenyl)propyl)amino)pyrimidin-2-yl)amino)-1H-pyrazol-1-yl)acetamide;
2-((5-chloro-2-((1-(2-hydroxyethyl)-1H-pyrazol-4-yl)amino)pyrimidin-4-yl)amino)-2-(2-fluorophenyl)-N,N-dimethylacetamide;
2-((5-chloro-2-((1-(2-hydroxyethyl)-1H-pyrazol-4-yl)amino)pyrimidin-4-yl)amino)-2-(2-fluorophenyl)-N-(2,2,2-trifluoroethyl)acetamide;
2-((5-chloro-2-((1-methyl-1H-pyrazol-4-yl)amino)pyrimidin-4-yl)amino)-2-(2,5-difluorophenyl)ethanol;
2-(4-((5-chloro-4-((1-(2,5-difluorophenyl)-2-hydroxyethyl)amino)pyrimidin-2-yl)amino)-1H-pyrazol-1-yl)-N-methylacetamide;
2-(4-((5-chloro-4-((2-hydroxy-1-(2-(trifluoromethyl)phenyl)ethyl)amino)pyrimidin-2-yl)amino)-1H-pyrazol-1-yl)-N-methylacetamide;
2-(4-((5-chloro-4-((1-(2,5-difluorophenyl)-2-hydroxyethyl)amino)pyrimidin-2-yl)amino)-1H-pyrazol-1-yl)acetamide;
(S)-2-(4-((4-((2-hydroxy-1-phenylethyl)amino)-5-methylpyrimidin-2-yl)amino)-1H-pyrazol-1-yl)-N-methylacetamide;
(S)-2-(4-((4-((2-hydroxy-1-phenylethyl)amino)-5-methylpyrimidin-2-yl)amino)-1H-pyrazol-1-yl)-N,N-dimethylacetamide;
3-(4-((5-chloro-4-(((S)-2-hydroxy-1-phenylethyl)amino)pyrimidin-2-yl)amino)-1H-pyrazol-1-yl)-1-methylpyrrolidin-2-one;
(S)-2-((5-chloro-2-((1-methyl-1H-pyrazol-4-yl)amino)pyrimidin-4-yl)amino)-2-(3-(1-methyl-1H-pyrazol-4-yl)phenyl)ethanol;
(S)-2-((5-chloro-2-((1-methyl-1H-pyrazol-4-yl)amino)pyrimidin-4-yl)amino)-2-(3-fluoro-5-(1-methyl-1H-pyrazol-4-yl)phenyl)ethanol;
(S)-4-(3-(1-((5-chloro-2-((1-methyl-1H-pyrazol-4-yl)amino)pyrimidin-4-yl)amino)-2-hydroxyethyl)phenyl)morpholin-3-one;
(S)-2-((5-chloro-2-((1-methyl-1H-pyrazol-4-yl)amino)pyrimidin-4-yl)amino)-2-(2,5-difluorophenyl)ethanol;
(S)-2-(4-((5-chloro-4-((1-(2,5-difluorophenyl)-2-hydroxyethyl)amino)pyrimidin-2-yl)amino)-1H-pyrazol-1-yl)acetamide;
(S)-2-(4-((5-chloro-4-((1-(2,5-difluorophenyl)-2-hydroxyethyl)amino)pyrimidin-2-yl)amino)-1H-pyrazol-1-yl)-N-methylacetamide;
(S)-2-((5-chloro-2-((1-methyl-1H-pyrazol-4-yl)amino)pyrimidin-4-yl)amino)-2-(3,5-difluorophenyl)ethanol;
(S)-2-((5-chloro-2-((1-(2-hydroxyethyl)-1H-pyrazol-4-yl)amino)pyrimidin-4-yl)amino)-2-(3,5-difluorophenyl)ethanol;
(S)-2-(4-((5-chloro-4-((1-(3,5-difluorophenyl)-2-hydroxyethyl)amino)pyrimidin-2-yl)amino)-1H-pyrazol-1-yl)acetamide;
(S)-2-(4-((5-chloro-4-((1-(3,5-difluorophenyl)-2-hydroxyethyl)amino)pyrimidin-2-yl)amino)-1H-pyrazol-1-yl)-N-methylacetamide;
(S)-2-(2,5-difluorophenyl)-2-((5-fluoro-2-((1-methyl-1H-pyrazol-4-yl)amino)pyrimidin-4-yl)amino)ethanol;
(S)-2-(2,5-difluorophenyl)-2-((5-fluoro-2-((1-(2-hydroxyethyl)-1H-pyrazol-4-yl)amino)pyrimidin-4-yl)amino)ethanol;
(S)-2-(4-((4-((1-(2,5-difluorophenyl)-2-hydroxyethyl)amino)-5-fluoropyrimidin-2-yl)amino)-1H-pyrazol-1-yl)-N-methylacetamide;
(S)-2-(3-(1-methyl-1H-pyrazol-4-yl)phenyl)-2-((5-methyl-2-((1-methyl-1H-pyrazol-4-yl)amino)pyrimidin-4-yl)amino)ethanol;
(S)-2-((5-fluoro-2-((1-methyl-1H-pyrazol-4-yl)amino)pyrimidin-4-yl)amino)-2-(3-fluoro-5-(1-methyl-1H-pyrazol-4-yl)phenyl)ethanol;
(S)-2-((5-chloro-2-((1-(2-hydroxyethyl)-1H-pyrazol-4-yl)amino)pyrimidin-4-yl)amino)-2-(3-(1-methyl-1H-pyrazol-4-yl)phenyl)ethanol;
(S)-2-((5-chloro-2-((1-(2-hydroxyethyl)-1H-pyrazol-4-yl)amino)pyrimidin-4-yl)amino)-2-(3-fluoro-5-(1-methyl-1H-pyrazol-4-yl)phenyl)ethanol;
(S)-2-(4-((5-fluoro-2-((1-(2-hydroxyethyl)-1H-pyrazol-4-yl)amino)pyrimidin-4-yl)amino)-2-(3-fluoro-5-(1-methyl-1H-pyrazol-4-yl)phenyl)ethanol;
(S)-2-(4-((5-chloro-4-((2-hydroxy-1-(3-(1-methyl-1H-pyrazol-4-yl)phenyl)ethyl)amino)pyrimidin-2-yl)amino)-1H-pyrazol-1-yl)-N-methylacetamide;
(S)-2-(4-((5-fluoro-4-((2-hydroxy-1-(3-(1-methyl-1H-pyrazol-4-yl)phenyl)ethyl)amino)pyrimidin-2-yl)amino)-1H-pyrazol-1-yl)-N-methylacetamide;
(S)-2-(4-((5-chloro-4-((1-(3-fluoro-5-(1-methyl-1H-pyrazol-4-yl)phenyl)-2-hydroxyethyl)amino)pyrimidin-2-yl)amino)-1H-pyrazol-1-yl)-N-methylacetamide;
(S)-2-(4-((5-fluoro-4-((1-(3-fluoro-5-(1-methyl-1H-pyrazol-4-yl)phenyl)-2-hydroxyethyl)amino)pyrimidin-2-yl)amino)-1H-pyrazol-1-yl)-N-methylacetamide;
(S)-2-(3,5-difluorophenyl)-2-((5-fluoro-2-((1-methyl-1H-pyrazol-4-yl)amino)pyrimidin-4-yl)amino)ethanol;
(S)-2-(3,5-difluorophenyl)-2-((5-fluoro-2-((1-(2-hydroxyethyl)-1H-pyrazol-4-yl)amino)pyrimidin-4-yl)amino)ethanol;
(S)-2-(4-((4-((1-(3,5-difluorophenyl)-2-hydroxyethyl)amino)-5-fluoropyrimidin-2-yl)amino)-1H-pyrazol-1-yl)acetamide;
(S)-2-(4-((4-((1-(3,5-difluorophenyl)-2-hydroxyethyl)amino)-5-fluoropyrimidin-2-yl)amino)-1H-pyrazol-1-yl)-N-methylacetamide;
(S)-4-(3-(1-((5-chloro-2-((1-methyl-1H-pyrazol-4-yl)amino)pyrimidin-4-yl)amino)-2-hydroxyethyl)-2-fluorophenyl)morpholin-3-one;
(S)-2-(2,5-difluorophenyl)-2-((5-methyl-2-((1-methyl-1H-pyrazol-4-yl)amino)pyrimidin-4-yl)amino)ethanol;
(S)-2-(3,5-difluorophenyl)-2-((5-methyl-2-((1-methyl-1H-pyrazol-4-yl)amino)pyrimidin-4-yl)amino)ethanol;
(S)-2-((5-chloro-2-((1-(2,2-difluoro ethyl)-1H-pyrazol-4-yl)amino)pyrimidin-4-yl)amino)-2-(2-fluorophenyl)ethanol;
(S)-2-((2-((1-(2,2-difluoro ethyl)-1H-pyrazol-4-yl)amino)-5-methylpyrimidin-4-yl)amino)-2-(2,5-difluorophenyl)ethanol;
(S)-2-((2-((1-(2,2-difluoro ethyl)-1H-pyrazol-4-yl)amino)-5-methylpyrimidin-4-yl)amino)-2-(3,5-difluorophenyl)ethanol;
2-((2-((1-(2,2-difluoroethyl)-1H-pyrazol-4-yl)amino)-5-methylpyrimidin-4-yl)amino)-2-(2,6-difluorophenyl)ethanol;
2-(4-((4-((1-(2,6-difluorophenyl)-2-hydroxyethyl)amino)-5-methylpyrimidin-2-yl)amino)-1H-pyrazol-1-yl)-1-morpholinoethanone;
(S)-2-(4-((5-chloro-4-((2-hydroxy-1-(pyridin-3-yl)ethyl)amino)pyrimidin-2-yl)amino)-1H-pyrazol-1-yl)-N-methylacetamide;
(S)-2-(3-bromophenyl)-2-((5-chloro-2-((1-methyl-1H-pyrazol-4-yl)amino)pyrimidin-4-yl)amino)ethanol;
(S)-2-(3-bromo-5-fluorophenyl)-2-((5-chloro-2-((1-methyl-1H-pyrazol-4-yl)amino)pyrimidin-4-yl)amino)ethanol;
(S)-2-(3-bromophenyl)-2-((5-methyl-2-((1-methyl-1H-pyrazol-4-yl)amino)pyrimidin-4-yl)amino)ethanol;
(S)-2-(3-bromo-5-fluorophenyl)-2-((5-fluoro-2-((1-methyl-1H-pyrazol-4-yl)amino)pyrimidin-4-yl)amino)ethanol;
(S)-2-(3-bromophenyl)-2-((5-chloro-2-((1-(2-hydroxyethyl)-1H-pyrazol-4-yl)amino)pyrimidin-4-yl)amino)ethanol;
(S)-2-(3-bromo-5-fluorophenyl)-2-((5-chloro-2-((1-(2-hydroxyethyl)-1H-pyrazol-4-yl)amino)pyrimidin-4-yl)amino)ethanol;
(S)-2-(3-bromo-5-fluorophenyl)-2-((5-fluoro-2-((1-(2-hydroxyethyl)-1H-pyrazol-4-yl)amino)pyrimidin-4-yl)amino)ethanol;
(S)-2-(4-((4-((1-(3-bromophenyl)-2-hydroxyethyl)amino)-5-chloropyrimidin-2-yl)amino)-1H-pyrazol-1-yl)-N-methylacetamide;
(S)-2-(4-((4-((1-(3-bromophenyl)-2-hydroxyethyl)amino)-5-fluoropyrimidin-2-yl)amino)-1H-pyrazol-1-yl)-N-methylacetamide;
(S)-2-(4-((4-((1-(3-bromo-5-fluorophenyl)-2-hydroxyethyl)amino)-5-chloropyrimidin-2-yl)amino)-1H-pyrazol-1-yl)-N-methylacetamide;
(S)-2-(4-((4-((1-(3-bromo-5-fluorophenyl)-2-hydroxyethyl)amino)-5-fluoropyrimidin-2-yl)amino)-1H-pyrazol-1-yl)-N-methylacetamide; and 2-(3-bromophenyl)-2-((5-chloro-2-((1-methyl-1H-pyrazol-4-yl)amino)pyrimidin-4-yl)amino)ethanol.

Where tautomerism, e.g. keto-enol tautomerism, of compounds of general formula (I) may occur, the individual forms, e.g. the keto and enol form, are comprised separately and together as mixtures in any ratio. The same applies for stereoisomers, e.g. enantiomers, cis/trans isomers, conformers and the like.

Isotopic labeled compounds (“isotopic derivatives”) of formula (I) are also within the scope of the present invention. Methods for isotope labeling are known in the art. Preferred isotopes are those of the elements H, C, N, O and S.

If desired, isomers can be separated by methods well known in the art, e.g. by liquid chromatography. The same applies for enantiomers by using e.g. chiral stationary phases. Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of formula (I) may be obtained from stereoselective synthesis using optically pure starting materials.

The compounds of formula (I) may exist in crystalline or amorphous form. Furthermore, some of the crystalline forms of the compounds of formula (I) may exist as polymorphs, which are included within the scope of the present invention. Polymorphic forms of compounds of formula (I) may be characterized and differentiated using a number of conventional analytical techniques, including, but not limited to, X-ray powder diffraction (XRPD) patterns, infrared (IR) spectra, Raman spectra, differential scanning calorimetry (DSC), thermogravimetric analysis (TGA) and solid state nuclear magnetic resonance (ssNMR).

In case the compounds according to formula (I) contain one or more acidic or basic groups, the invention also comprises their corresponding pharmaceutically or toxicologically acceptable salts, in particular their pharmaceutically utilizable salts. Thus, the compounds of the formula (I) which contain acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the formula (I) which contain one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the formula (I) simultaneously contain acidic and basic groups in the molecule, the invention also includes, in addition to the salt forms mentioned, inner salts or betaines (zwitterions). The respective salts according to the formula (I) can be obtained by customary methods which are known to the person skilled in the art like, for example by contacting these with an organic or inorganic acid or base in a solvent or dispersant, or by anion exchange or cation exchange with other salts. The present invention also includes all salts of the compounds of the formula (I) which, owing to low physiological compatibility, are not directly suitable for use in pharmaceuticals but which can be used, for example, as intermediates for chemical reactions or for the preparation of pharmaceutically acceptable salts.

Throughout the invention, the term “pharmaceutically acceptable” means that the corresponding compound, carrier or molecule is suitable for administration to humans. Preferably, this term means approved by a regulatory agency such as the EMEA (Europe) and/or the FDA (US) and/or any other national regulatory agency for use in animals, preferably in humans.

The present invention furthermore includes all solvates of the compounds according to the invention.

According to the present invention “JAK” comprises all members of the JAK family (e.g. JAK1, JAK2, JAK3, and TYK2).

According to the present invention, the expression “JAK1” or “JAK1 kinase” means “Janus kinase 1”. The human gene encoding JAK1 is located on chromosome 1p31.3.

According to the present invention, the expression “JAK2” or “JAK2 kinase” means “Janus kinase 2”. The human gene encoding JAK2 is located on chromosome 9p24.

According to the present invention, the expression “JAK3” or “JAK3 kinase” means “Janus kinase 3”. The gene encoding JAK3 is located on human chromosome 19p13.1 and it is predominantly in hematopoietic cells. JAK3 is a cytoplasmic protein tyrosine kinase that associates with the gamma-chain of the interleukin 2 (IL-2) receptor. This chain also serves as a component for the receptors of several lymphotropic cytokines, including interleukins IL-4, IL-7, IL-9, IL-15 and IL-21 (Schindler et al., 2007. J. Biol. Chem. 282(28):20059-63). JAK3 plays a key role in the response of immune cells to cytokines, especially in mast cells, lymphocytes and macrophages Inhibition of JAK3 has shown beneficial effects in the prevention of transplant rejection (Changelian et al., 2003, Science 302(5646):875-888).

Moreover, according to the present invention, the expression “JAK3” or “JAK3 kinase” includes mutant forms of JAK3, preferably JAK3 mutants found in acute megakaryoblastic leukemia (AMKL) patients. More preferred, these mutants are single amino acid mutations. Activating JAK3 mutations were observed in acute megakaryoblastic leukemia (AMKL) patients (Walters et al., 2006. Cancer Cell 10(1):65-75). Therefore, in a preferred embodiment, the expression “JAK” also includes a JAK3 protein having a V7221 or P132T mutation.

According to the present invention, the expression “TYK2” or “TYK2 kinase” means “Protein-Tyrosine kinase 2”. The JAK3 and TYK2 genes are clustered on chromosome 19p13.1 and 19p13.2, respectively.

As shown in the examples, compounds of the invention were tested for their selectivity for JAK3 over JAK2 kinases. As shown, all tested compounds bind JAK3 more selectively than, JAK2 (see table 8 below).

Consequently, the compounds of the present invention are considered to be useful for the prevention or treatment of diseases and disorders associated with JAK, for example immunological, inflammatory, autoimmune, or allergic disorders, transplant rejection, Graft-versus-Host-Disease or proliferative diseases such as cancer.

In a preferred embodiment, the compounds of the present invention are selective JAK3 inhibitors.

Equally preferred are dual JAK1/JAK3 inhibitors.

The compounds of the present invention may be further characterized by determining whether they have an effect on JAK3, for example on its kinase activity (Changelian et al., 2003, Science 302(5646):875-888 and online supplement; Yang et al., 2007. Bioorg. Med. Chem. Letters 17(2): 326-331).

Briefly, JAK3 kinase activity can be measured using a recombinant GST-JAK3 fusion protein comprising the catalytic domain (JH1 catalytic domain). JAK3 kinase activity is measured by ELISA as follows: Plates are coated overnight with a random L-glutamic acid and tyrosine co-polymer (4:1; 100 μg/ml) as a substrate. The plates are washed and recombinant JAK3 JHLGST protein (100 ng/well) with or without inhibitors is incubated at room temperature for 30 minutes. The a HPR-conjugated PY20 anti-phosphotyrosine antibody (ICN) is added and developed by TMB (3,3′,5,5′-tetramethylbenzidine) (Changelian et al., 2003, Science 302 (5646): 875-888 and online supplement).

A cell-based assays (TF-1 cell proliferation) was described to assess the inhibitory activity of small molecule drugs toward JAK2 or JAK3-dependent signal transduction (Chen et al., 2006. Bioorg. Med. Chem. Letters 16(21): 5633-5638).

The present invention provides pharmaceutical compositions comprising a compound of formula (I) or a pharmaceutically acceptable salt or isotopic derivative thereof as active ingredient together with a pharmaceutically acceptable carrier, optionally in combination with one or more other pharmaceutical compositions.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

A pharmaceutical composition of the present invention may comprise one or more additional compounds as active ingredients like one or more compounds of formula (I) not being the first compound in the composition or other JAK inhibitors. Further bioactive compounds may be steroids, leukotriene antagonists, cyclosporine or rapamycin.

The compounds of the present invention or pharmaceutically acceptable salt(s) or isotopic derivative(s) thereof and the other pharmaceutically active agent(s) may be administered together or separately and, when administered separately, this may occur separately or sequentially in any order. When combined in the same formulation it will be appreciated that the two compounds must be stable and compatible with each other and the other components of the formulation. When formulated separately they may be provided in any convenient formulation, conveniently in such manner as are known for such compounds in the art.

It is further included within the present invention that the compound of formula (I), or a pharmaceutically acceptable salt or isotopic derivative thereof, or a pharmaceutical composition comprising a compound of formula (I) is administered in combination with another drug or pharmaceutically active agent and/or that the pharmaceutical composition of the invention further comprises such a drug or pharmaceutically active agent.

In this context, the term “drug or pharmaceutically active agent” includes a drug or pharmaceutical agent that will elicit the biological or medical response of a tissue, system, animal or human that is being sought, for instance, by a researcher or clinician.

“Combined” or “in combination” or “combination” should be understood as a functional coadministration, wherein some or all compounds may be administered separately, in different formulations, different modes of administration (for example subcutaneous, intravenous or oral) and different times of administration. The individual compounds of such combinations may be administered either sequentially in separate pharmaceutical compositions as well as simultaneously in combined pharmaceutical compositions.

For example, in rheumatoid arthritis therapy, combination with other chemotherapeutic or antibody agents is envisaged. Suitable examples of pharmaceutically active agents which may be employed in combination with the compounds of the present invention and their salts for rheumatoid arthritis therapy include: immunosuppresants such as amtolmetin guacil, mizoribine and rimexolone; anti-TNFα agents such as etanercept, infliximab, Adalimumab, Anakinra, Abatacept, Rituximab; tyrosine kinase inhibitors such as leflunomide; kallikrein antagonists such as subreum; interleukin 11 agonists such as oprelvekin; interferon beta 1 agonists; hyaluronic acid agonists such as NRD-101 (Aventis); interleukin 1 receptor antagonists such as anakinra; CD8 antagonists such as amiprilose hydrochloride; beta amyloid precursor protein antagonists such as reumacon; matrix metalloprotease inhibitors such as cipemastat and other disease modifying anti-rheumatic drugs (DMARDs) such as methotrexate, sulphasalazine, cyclosporin A, hydroxychoroquine, auranofin, aurothioglucose, gold sodium thiomalate and penicillamine.

In particular, the treatment defined herein may be applied as a sole therapy or may involve, in addition to the compounds of the invention, conventional surgery or radiotherapy or chemotherapy. Accordingly, the compounds of the invention can also be used in combination with existing therapeutic agents for the treatment proliferative diseases such as cancer. Suitable agents to be used in combination include:

(i) antiproliferative/antineoplastic drugs and combinations thereof, as used in medical oncology such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and taxotere); and topoisomerase inhibitors (for example epipodophyllotoxins like etoposide and teniposide, amsacrine, topotecan and camptothecins);

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxy-quinazoline (AZD0530) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825), and metalloproteinase inhibitors like marimastat and inhibitors of urokinase plasminogen activator receptor function);

(iv) inhibitors of growth factor function: for example such inhibitors include growth factor antibodies and growth factor receptor antibodies (for example the anti-erbB2 antibody trastuzumab [Herceptin™] and the anti-erbB1 antibody cetuximab [C225]); such inhibitors also include, for example, tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD 1839), Λ/-(3-ethynylphenyl)-6,7-bis(2-methoxyethoxy)quinazolin-4-amine (erlotinib, OSI-774) and 6-acrylamido-Λ/-(3-chloro-4-fluorophenyl)-7-(3-morpholinopropoxy)-quinazolin-4-amine (CI 1033) and erbB2 tyrosine kinase inhibitors such as lapatinib), inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)) and inhibitors of cell signalling through MEK and/or Akt kinases;

(v) antiangiogenic agents such as those which inhibit the effects of vascular endothelial growth factor, for example the anti-vascular endothelial cell growth factor antibody bevacizumab (Avastin™) and VEGF receptor tyrosine kinase inhibitors such as 4-(4-bromo-2-fiuoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), and compounds that work by other mechanisms (for example linomide, inhibitors of integrin αvβ3 function and angio statin);

(vi) vascular damaging agents such as combretastatin A4 and compounds disclosed in International Patent Application WO 99/02166;

(vii) antisense therapies, for example those which are directed to the targets listed above, such as ISIS 2503, an anti-ras antisense agent;

(viii) gene therapy approaches, including approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and (ix) immunotherapeutic approaches, including ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Further combination treatments are described in WO-A 2009/008992 and WO-A 2007/107318), incorporated herein by reference.

Accordingly, the individual compounds of such combinations may be administered either sequentially in separate pharmaceutical compositions as well as simultaneously in combined pharmaceutical compositions.

The pharmaceutical compositions of the present invention include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of formula (I) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally, for example, as liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula (I) may also be administered parenterally. Solutions or suspensions of these active compounds can be prepared in water suitably mixed with a surfactant such as hydroxypropyl-cellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of formula (I) are administered orally.

The effective dosage of active ingredient employed may vary depending on the particular compound employed, the mode of administration, the condition being treated and the severity of the condition being treated. Such dosage may be ascertained readily by a person skilled in the art.

A therapeutically effective amount of a compound of the present invention will normally depend upon a number of factors including, for example, the age and weight of the animal, the precise condition requiring treatment and its severity, the nature of the formulation, and the route of administration. However, an effective amount of a compound of formula (I) for the treatment of an inflammatory disease, for example rheumatoid arthritis (RA), will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a pharmaceutically acceptable salt, prodrug or metabolite thereof, may be determined as a proportion of the effective amount of the compound of formula (I) per se. It is envisaged that similar dosages would be appropriate for treatment of the other conditions referred to above.

As used herein, the term “effective amount” means that amount of a drug or pharmaceutical agent that will elicit the biological or medical response of a tissue, system, animal or human that is being sought, for instance, by a researcher or clinician.

Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Another aspect of the present invention is a compound of the present invention or a pharmaceutically acceptable salt or isotopic derivative thereof for use as a medicament.

Another aspect of the present invention is a compound of the present invention or a pharmaceutically acceptable salt or isotopic derivative thereof for use in a method of treating or preventing a disease or disorder associated with JAK.

In the context of the present invention, a disease or disorder associated with JAK is defined as a disease or disorder where JAK is involved.

In a preferred embodiment, wherein the diseases or disorder is associated with JAK is an immunological, inflammatory, autoimmune, or allergic disorder or disease of a transplant rejection or a Graft-versus host disease.

Consequently, another aspect of the present invention is a compound or a pharmaceutically acceptable salt thereof of the present invention for use in a method of treating or preventing an immunological, inflammatory, autoimmune, or allergic disorder or disease of a transplant rejection or a Graft-versus host disease.

Inflammation of tissues and organs occurs in a wide range of disorders and diseases and in certain variations, results from activation of the cytokine family of receptors. Exemplary inflammatory disorders associated with activation of JAK include, in a non-limiting manner, skin inflammation due radiation exposure, asthma, allergic inflammation and chronic inflammation.

According to the present invention, an autoimmune disease is a disease which is at least partially provoked by an immune reaction of the body against own components, for example proteins, lipids or DNA. Examples of organ-specific autoimmune disorders are insulin-dependent diabetes (Type I) which affects the pancreas, Hashimoto's thyroiditis and Graves' disease which affect the thyroid gland, pernicious anemia which affects the stomach, Cushing's disease and Addison's disease which affect the adrenal glands, chronic active hepatitis which affects the liver; polycystic ovary syndrome (PCOS), celiac disease, psoriasis, inflammatory bowel disease (IBD) and ankylosing spondylitis. Examples of non-organ-specific autoimmune disorders are rheumatoid arthritis, multiple sclerosis, systemic lupus and myasthenia gravis.

Type I diabetes ensues from the selective aggression of autoreactive T-cells against insulin secreting beta-cells of the islets of Langerhans. Targeting JAK3 in this disease is based on the observation that multiple cytokines that signal through the JAK pathway are known to participate in the T-cell mediated autoimmune destruction of beta-cells. Indeed, a JAK3 inhibitor, JANEX-1 was shown to prevent spontaneous autoimmune diabetes development in the NOD mouse model of type I diabetes.

In a preferred embodiment, the autoimmune disease is selected from the group consisting of rheumatoid arthritis (RA), inflammatory bowel disease (IBD; Crohn's disease and ulcerative colitis), psoriasis, systemic lupus erythematosus (SLE), and multiple sclerosis (MS).

Rheumatoid arthritis (RA) is a chronic progressive, debilitating inflammatory disease that affects approximately 1% of the world's population. RA is a symmetric polyarticular arthritis that primarily affects the small joints of the hands and feet. In addition to inflammation in the synovium, the joint lining, the aggressive front of tissue called pannus invades and destroys local articular structures (Firestein 2003, Nature 423:356-361).

Inflammatory bowel disease (IBD) is characterized by a chronic relapsing intestinal inflammation. IBD is subdivided into Crohn's disease and ulcerative colitis phenotypes. Crohn disease involves most frequently the terminal ileum and colon, is transmural and discontinuous. In contrast, in ulcerative colitis, the inflammation is continuous and limited to rectal and colonic mucosal layers. In approximately 10% of cases confined to the rectum and colon, definitive classification of Crohn's disease or ulcerative colitis cannot be made and are designated ‘indeterminate colitis.’ Both diseases include extraintestinal inflammation of the skin, eyes, or joints. Neutrophil-induced injuries may be prevented by the use of neutrophils migration inhibitors (Asakura et al., 2007, World J Gastroenterol. 13(15):2145-9).

Psoriasis is a chronic inflammatory dermatosis that affects approximately 2% of the population. It is characterized by red, scaly skin patches that are usually found on the scalp, elbows, and knees, and may be associated with severe arthritis. The lesions are caused by abnormal keratinocyte proliferation and infiltration of inflammatory cells into the dermis and epidermis (Scholl et al., 2005, New Engl. J. Med. 352:1899-1912).

Systemic lupus erythematosus (SLE) is a chronic inflammatory disease generated by T cell-mediated B-cell activation, which results in glomerulonephritis and renal failure. Human SLE is characterized at early stages by the expansion of long-lasting autoreactive CD4+ memory cells (D′Cruz et al., 2007, Lancet 369(9561):587-596).

Multiple sclerosis (MS) is an inflammatory and demyelating neurological disease. It has bee considered as an autoimmune disorder mediated by CD4+ type 1 T helper cells, but recent studies indicated a role of other immune cells (Hemmer et al., 2002, Nat. Rev. Neuroscience 3, 291-301).

Mast cells express JAK3 and JAK3 is a key regulator of the IgE mediated mast cell responses including the release of inflammatory mediators. JAK3 was shown to be a valid target in the treatment of mast cell mediated allergic reaction. Allergic disorders associated with mast cell activation include Type I immediate hypersensitivity reactions such as allergic rhinitis (hay fever), allergic urticaria (hives), angioedema, allergic asthma and anaphylaxis, for example anaphylatic shock. These disorders may be treated or prevented by inhibition of JAK3 activity, for example, by administration of a JAK3 inhibitor according to the present invention.

Transplant rejection (allograft transplant rejection) includes, without limitation, acute and chronic allograft rejection following for example transplantation of kidney, heart, liver, lung, bone marrow, skin and cornea. It is known that T cells play a central role in the specific immune response of allograft rejection. Hyperacute, acute and chronic organ transplant rejection may be treated. Hyperacute rejection occurs within minutes of transplantation. Acute rejection generally occurs within six to twelve months of the transplant. Hyperacute and acute rejections are typically reversible where treated with immunosuppressant agents. Chronic rejection, characterized by gradual loss of organ function, is an ongoing concern for transplant recipients because it can occur anytime after transplantation.

Graft-versus-host disease (GVDH) is a major complication in allogeneic bone marrow transplantation (BMT). GVDH is caused by donor T cells that recognize and react to recipient differences in the histocompatibility complex system, resulting in significant morbidity and mortality. JAK3 plays a key role in the induction of GVHD and treatment with a JAK3 inhibitor, JANEX-1, was shown to attenuate the severity of GVHD (reviewed in Cetkovic-Cvrlje and Ucken, 2004).

In a preferred embodiment, the inflammatory disease is an eye disease.

Dry eye syndrome (DES, also known as keratoconjunctivitis sicca) is one of the most common problems treated by eye physicians. Sometimes DES is referred to as dysfunctional tear syndrome (Jackson, 2009. Canadian Journal Ophthalmology 44(4), 385-394). DES affects up to 10% of the population between the ages of 20 to 45 years, with this percentage increasing with age. Although a wide variety of artificial tear products are available, these products provide only transitory relief of symptoms. As such, there is a need for agents, compositions and therapeutic methods to treat dry eye.

As used herein, “dry eye disorder” is intended to encompass the disease states summarized in a recent official report of the Dry Eye Workshop (DEWS), which defined dry eye as “a multifactorial disease of the tears and ocular surface that results in symptoms of discomfort, visual disturbance, and tear film instability with potential damage to the ocular surface. It is accompanied by increased osmolality of the tear film and inflammation of the ocular surface.” (Lemp, 2007. “The Definition and Classification of Dry Eye Disease: Report of the Definition and Classification Subcommittee of the International Dry Eye Workshop”, The Ocular Surface, 5(2), 75-92). Dry eye is also sometimes referred to as keratoconjunctivitis sicca. In some embodiments, the treatment of the dry eye disorder involves ameliorating a particular symptom of dry eye disorder, such as eye discomfort, visual disturbance, tear film instability, tear hyperosmolarity, and inflammation of the ocular surface.

Uveitis is the most common form of intraocular inflammation and remains a significant cause of visual loss. Current treatments for uveitis employs systemic medications that have severe side effects and are globally immunosuppressive. Clinically, chronic progressive or relapsing forms of non-infectious uveitis are treated with topical and/or systemic corticosteroids. In addition, macrolides such as cyclosporine and rapamycin are used, and in some cases cytotoxic agents such as cyclophosphamide and chlorambucil, and antimetabolites such as azathioprine, methotrexate, and leflunomide (Srivastava et al., 2010. Uveitis: Mechanisms and recent advances in therapy. Clinica Chimica Acta, doi:10.1016/j.cca.2010.04.017).

Further eye diseases, combination treatments and route of administration are described for example in WO-A 2010/039939, which is hereby incorporated herein by reference.

In a further preferred embodiment, the disease or disorder associated with JAK is a proliferative disease, especially cancer.

Diseases and disorders associated especially with JAK are proliferative disorders or diseases, especially cancer.

Therefore, another aspect of the present invention is a compound or a pharmaceutically acceptable salt or isotopic derivative thereof of the present invention for use in a method of treating or preventing a proliferative disease, especially cancer.

Cancer comprises a group of diseases characterized by uncontrolled growth and spread of abnormal cells. All types of cancers generally involve some abnormality in the control of cell growth, division and survival, resulting in the malignant growth of cells. Key factors contributing to said malignant growth of cells are independence from growth signals, insensitivity to anti-growth signals, evasion of apoptosis, limitless replicative potential, sustained angiogenesis, tissue invasion and metastasis, and genome instability (Hanahan and Weinberg, 2000. The Hallmarks of Cancer. Cell 100, 57-70).

Typically, cancers are classified as hematological cancers (for example leukemias and lymphomas) and solid cancers such as sarcomas and carcinomas (for example cancers of the brain, breast, lung, colon, stomach, liver, pancreas, prostate, ovary).

The JAK inhibitors of the present invention may also useful in treating certain malignancies, including skin cancer and hematological malignancy such as lymphomas and leukemias.

Especially cancers in which the JAK-STAT signal transduction pathway is activated, for example due to activation of JAK3 are expected to respond to treatment with JAK3 inhibitors. Examples of cancers harboring JAK3 mutations are acute megakaryoblastic leukemia (AMKL) (Walters et al., 2006. Cancer Cell 10(1):65-75) and breast cancer (Jeong et al., 2008. Clin. Cancer Res. 14, 3716-3721).

Proliferative diseases or disorders comprise a group of diseases characterized by increased cell multiplication as observed in myeloprolifetative disorders (MPD) such as polycythemia vera (PV).

Yet another aspect of the present invention is the use of a compound of the present invention or a pharmaceutically acceptable salt or isotopic derivative thereof for the manufacture of a medicament for the treatment or prophylaxis of diseases and disorders associated with JAK.

Yet another aspect of the present invention is the use of a compound of the present invention or a pharmaceutically acceptable salt or isotopic derivative thereof for the manufacture of a medicament for treating or preventing an immunological, inflammatory, autoimmune, or allergic disorder or disease or a transplant rejection or a Graft-versus host disease.

Yet another aspect of the present invention is the use of a compound of the present invention or a pharmaceutically acceptable salt or isotopic derivative thereof for the manufacture of a medicament for treating or preventing a proliferative disease, especially cancer.

In the context of these uses of the invention, diseases and disorders associated with JAK are as defined above.

Yet another aspect of the present invention is a method for treating, controlling, delaying or preventing in a mammalian patient in need thereof one or more conditions selected from the group consisting of diseases and disorders associated with JAK, wherein the method comprises the administration to said patient a therapeutically effective amount of a compound according to present invention or a pharmaceutically acceptable salt or isotopic derivative thereof.

Yet another aspect of the present invention is a method for treating, controlling, delaying or preventing in a mammalian patient in need thereof one or more conditions selected from the group consisting of an immunological, inflammatory, autoimmune, or allergic disorder or disease or a transplant rejection or a Graft-versus host disease, wherein the method comprises the administration to said patient a therapeutically effective amount of a compound according to present invention or a pharmaceutically acceptable salt or isotopic derivative thereof.

Yet another aspect of the present invention is a method for treating, controlling, delaying or preventing in a mammalian patient in need thereof a proliferative disease, especially cancer, wherein the method comprises the administration to said patient a therapeutically effective amount of a compound according to present invention or a pharmaceutically acceptable salt or isotopic derivative thereof.

In the context of these methods of the invention, diseases and disorders associated with JAK are as defined above.

As used herein, the term “treating” or “treatment” is intended to refer to all processes, wherein there may be a slowing, interrupting, arresting, or stopping of the progression of a disease, but does not necessarily indicate a total elimination of all symptoms.

All embodiments discussed above with respect to the pharmaceutical composition of the invention also apply to the above mentioned first or second medical uses or methods of the invention.

General methods for the preparation of compounds of the present invention are known (like from WO 2006/117560 A1) in the art. In the following experimental section preparation methods are described which also can be used in analogues methods using methods known to the skilled person in the art, especially methods for protecting reactive functional groups or activating functional groups.

### Analytical Methods

LCMS was carried out on an Agilent 1100, UPLCMS was carried out on a Waters UPBINARY, water and ACN (0.1% formic acid—low pH, 0.1% ammonia—high pH) with an injection volume of 3 μL. Wavelengths were 254 and 210 nm

Method A UPLC Low pH,

Method B UPLC High pH

Column: Waters Acquity UPLC BEH C18, 30×2.1 mm, 1.7 mm. Flow rate 0.5 mL/min

TABLE 1

| Time (min) | Water (%) | ACN (%) |
|---|---|---|
| 0.00 | 95.0 | 5.0 |
| 0.20 | 95.0 | 5.0 |
| 1.00 | 5.0 | 95.0 |
| 1.50 | 5.0 | 95.0 |
| 1.70 | 95.0 | 5.0 |
| 2.70 | 95.0 | 5.0 |

Method C LCMS Low pH

Method D LCMS High pH

Column: Phenomenex Gemini-C18, 3×30 mm, 3 microns. Flow rate: 1.2 mL/min

TABLE 2

| Time (min) | Water (%) | ACN (%) |
|---|---|---|
| 0.00 | 95.0 | 5.0 |
| 3.00 | 5.0 | 95.0 |
| 4.50 | 5.0 | 95.0 |
| 4.60 | 95.0 | 5.0 |

| | |
|---|---|
| 5.00 | STOP |

Method E LCMS Low pH 16 minute

Column: Phenomenex Gemini-C18, 4.6×150 mm, 5 microns. Flow rate: 1.0 mL/min. LOW pH

TABLE 3

| | water | |
|---|---|---|
| Time (min) | (%) | ACN (%) |
| 0.00 | 95.0 | 5.0 |
| 11.00 | 5.0 | 95.0 |
| 13.00 | 5.0 | 95.0 |
| 13.01 | 95.0 | 5.0 |
| 16.00 | 95.0 | 5.0 |

Method F: UPLC High pH 6 minute

Column: Waters Acquity UPLC BEH C18, 2.1×50 mm, 1.7 microns. Flow rate: 0.5 mL/min. LOW pH

TABLE 4

| | Water | |
|---|---|---|
| Time (min) | (%) | ACN (%) |
| 0.00 | 95.0 | 5.0 |
| 0.20 | 95.0 | 5.0 |
| 4.20 | 5.0 | 95.0 |
| 4.70 | 5.0 | 95.0 |
| 4.75 | 95.0 | 5.0 |
| 6.00 | 95.0 | 5.0 |

| | |
|---|---|
| ACN | Acetonitrile |
| Ar | Aryl |
| Aq | Aqueous |
| Boc | Tert-Butoxycarbonyl |
| brs | Broad singlet |
| d | Doublet |
| dd | Double doublets |
| DCM | Dichloromethane |
| DIPEA | Diisopropylethylamine |
| DMF | N,N′-Dimethylformamide |
| DMSO | N,N′- dimethylsulfoxide |
| DP | Drug pulldown |
| DTT | Dithiothreitol |
| EDTA | Ethylenediaminetetraacetic acid |
| EtOAc | Ethyl acetate |
| eq | Equivalents |
| g | Grams |
| h | Hours |
| HATU | N,N,N′,N′-Tetramethyl-O-(7-azabenzotriazol-1- |
| | yl)uronium hexafluorophosphate |
| HCl | Hydrochloric acid |
| H₂O | Water |
| HPLC | High performance liquid chromatography |
| Hz | Hertz |
| IC₅₀ | 50% Maximium inhibition concentration |
| IPA | Propan-2-ol |
| iPr | Isopropyl |
| J | Coupling Constant |
| L | Litres |
| LC-MS | Liquid chromatography mass spectroscopy |
| m | Multiplet |
| M | Molar |
| MeOH | Methanol |
| mg | Milligrams |
| MgSO₄ | Magnesium Sulphate |
| Min | Minutes |
| mL | Millilitres |
| Mm | Millimetres |
| Mmol | Millimoles |
| mol % | Molar percent |
| μL | Microlitres |
| NaHCO₃ | Sodium Hydrogen Carbonate |
| Nm | Nanometres |
| PBS | Phosphate buffered saline |
| Rpm | Revolutions per minute |
| RT | Retention time |
| sat. | Saturated |
| s | Singlet |
| td | Triplet doublets |
| t | Triplet |

### EXPERIMENTAL

A brief description of exemplary routes for the synthesis of compounds of the present invention is given below in Schemes A1 to A4.

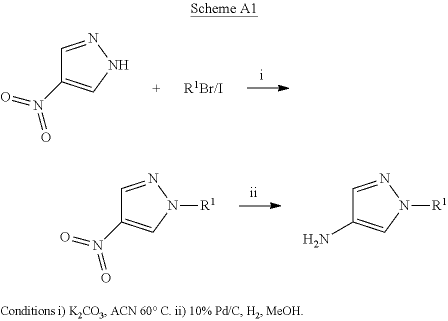

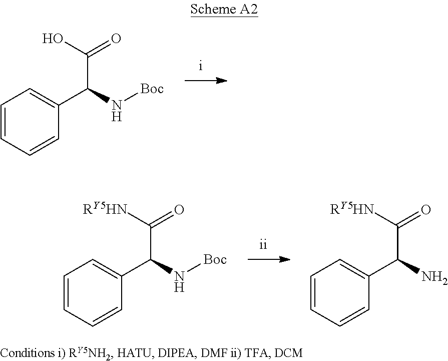

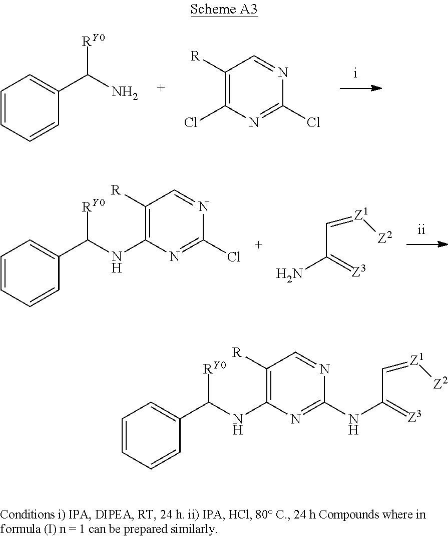

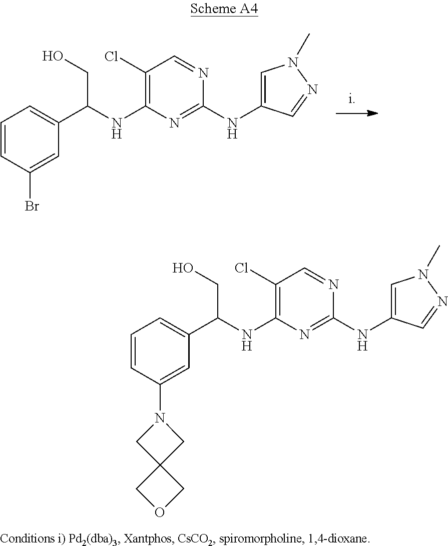 [SMILES: Cn1cc(Nc2ncc(Cl)c(NC(CO)c3cccc(Br)c3)n2)cn1 ; Cn1cc(Nc2ncc(Cl)c(NC(CO)c3cccc(N4CC5(COC5)C4)c3)n2)cn1]

### General Procedure for the Synthesis of 4-Amino-1-N-alkylated-pyrazoles

### Scheme A1, Step 1

A solution of 4-nitropyrazole (1.0 eq), potassium carbonate (2.0 eq) and the alkylating reagent (1.1 eq) in acetonitrile (10 vols) was heated at 60° C. for 18 h. After cooling to room temperature the mixture was diluted with EtOAc and washed with water. The organic phase was collected, dried (MgSO₄) and concentrated in vacuo.

### Step 2

The crude nitro residue was dissolved in methanol (50 vols), palladium on carbon (10% wt) was added and the reaction was stirred under an atmosphere of H₂ for 18 h. The resulting mixture was filtered through Celite and the filtrate concentrated in vacuo to give the desired product.

### General Procedure for the Preparation of Amide Substituted Benzylamines

### Scheme A2, Step 1

To a stirred solution of N-(tert-butoxycarbonyl)-L-2-phenylglycine (1 eq) in DMF (2 ml) was added the relevant amine (1.1 eq), HATU (1.3 eq) and DIPEA (2 eq). The reaction was then stirred for 1 h at room temperature. The reaction was diluted with DCM and washed with water, dried using a hydrophobic frit and concentrated in vacuo. Reverse phase flash chromatography (30 g C18 column, 5%-95% ACN with formic acid in water with formic acid) gave the desired amide.

### Scheme A2, Step 2

The BOC-aminomethylamide was stirred for 1 h at room temperature in TFA/DCM (1:4). The reaction was loaded on to a tosic acid SPE cartridge and after washing the product was eluted with 2 N ammonia in methanol. The organics were removed in vacuo to give the desired product.

### Scheme A3, Step 1

General synthesis of intermediate N-(2-chloro-5-fluoropyrimidin-4-yl)-benzylamines

To a solution of 2,4-dichloro-5-fluoropyrimidine (1.0 eq) and DIPEA (2.0 eq) in propan-2-ol (10 vols) at 0° C. was added drop wise a substituted benzylamine (1.0 eq), the resultant mixture was stirred overnight at room temperature. The reaction was then diluted with EtOAc (20 vols) and water (20 vols) and brine (10 vols). The organic layer was collected, dried (MgSO₄) concentrated under reduced pressure.

### General synthesis of intermediate N-(2-chloro-5-chloropyrimidin-4-yl)-benzylamines

To a solution of 2,4,5-trichloropyrimidine (1.0 eq) and DIPEA (2.0 eq) in propan-2-ol (10 vols) at 0° C. was added drop wise a substituted benzylamine (1.0 eq), the resultant mixture was stirred overnight at room temperature. The reaction was then diluted with EtOAc (20 vols) and water (20 vols) and brine (10 vols). The organic layer was collected dried (MgSO₄) concentrated under reduced pressure to provide the desired product.

### General synthesis of intermediate N-(2-chloro-5-methylpyrimidin-4-yl)-benzylamines

To a solution of 2,4-dichloro-5-methylpyrimidine (1.0 eq) and DIPEA (2.0 eq) in propan-2-ol (10 vols) at 0° C. was added drop wise a substituted benzylamine (1.0 eq), the resultant mixture was stirred overnight at room temperature. The reaction was then diluted with EtOAc (20 vols) and water (20 vols) and brine (10 vols). The organic layer was collected dried (MgSO₄) concentrated under reduced pressure to provide the desired product.

### General Synthesis of Test Compounds

### Scheme A3, Step 2

A mixture of N-(2-chloro-5-chloropyrimidin-4-yl)-benzylamine, substituted 1H-pyrazol-4-amine (1.0 eq) and 4M HC1 in dioxane (0.1 eq) were stirred at 80° C. for 18 h in propan-2-ol (5 vols). The reaction was then diluted with EtOAc (20 vols) and NaHCO₃ (10 vols) the organic phase was collected dried (MgSO₄) and evaporated to provide the desired product. The products were further purified by Flash chromatography (EtOAc/Petrol) or HPLC where necessary.

### General Procedure for the Preparation of Amine Substituted Test Compounds

### Scheme A4

A suspension of the aryl bromide (1.0 eq), spiromorpholine (1.5 eq), Pd₂(dba)₃ (0.01 eq), XANTPHOS (0.05 eq) and cesium carbonate (3.0 eq) was refluxed in degassed 1,4-dioxane (10 vols) overnight. The mixture was cooled and filtered the filtrate was passed though PS-SH cartridge then solvent removed in vacuo. Purification can be achieved by reverse phase chromatography.

A more detailed description of exemplary routes for the synthesis of compounds of the present invention is given below.

### General Procedure for the Synthesis of 4-Amino-1-N-alkylated-pyrazoles 1.3

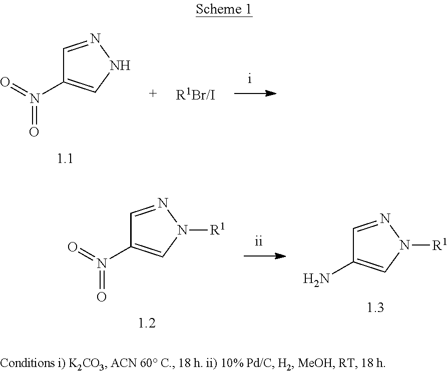

### Step 1

A solution of 4-nitropyrazole (1.0 eq), potassium carbonate (2.0 eq) and the alkylating reagent (1.1 eq) in ACN (10 vols) was heated at 60° C. for 18 h. After cooling to room temperature the mixture was diluted with EtOAc and washed with water. The organic phase was collected, dried (MgSO₄) and concentrated in vacuo.

### Step 2

The alkylated nitropyrazole 1.2 was dissolved in MeOH (50 vols), palladium on carbon (10% wt) was added and the reaction was stirred under an atmosphere of H₂ for 18 h. The resulting mixture was filtered through Celite and the filtrate concentrated in vacuo to give the desired product.

### General Procedure for the Preparation of Amide Substituted Benzylamines 2.3

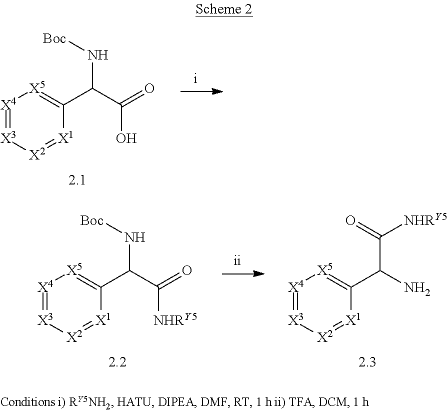

### Step 1

To a stirred solution of 2.1 (1.0 eq) in DMF (2 mL) was added the relevant amine R^{Y5}NH₂ (1.1 eq), HATU (1.3 eq) and DIPEA (2.0 eq). The reaction was then stirred for 1 h at room temperature. The reaction was diluted with DCM and washed with water, dried using a hydrophobic frit and concentrated in vacuo. Reverse phase flash chromatography (30 g C18 column, 5%-95% ACN with formic acid in water with formic acid) gave the desired amide 2.2.

### Step 2

Amide 2.2 was stirred for 1 h at room temperature in TFA/DCM (1:4). The reaction was loaded on to a tosic acid SPE cartridge and after washing with MeOH, the product was eluted with 2 N ammonia in MeOH. The organics were removed in vacuo to give the desired product 2.3.

### General Procedure for the Preparation of Morpholinone Substituted Benzylamines 3.2

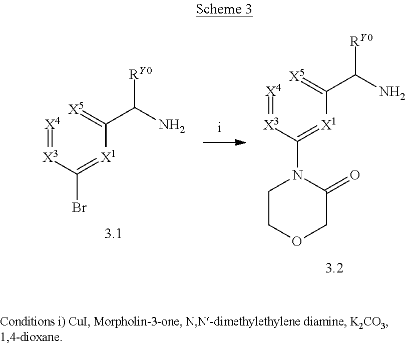

The aryl bromide 3.1 (1.0 eq), morpholin-3-one (1.25 eq), CuI (0.2 eq), N,N′-dimethylethylene diamine (0.4 eq) and K₂CO₃ (2.0 eq) were heated to 110° C. in dioxane for 18 h. The reaction mixture was cooled, diluted with water and EtOAC. The organic layer was rinsed (water, brine), dried (MgSO₄) and concentrated to yield 3.2

### General Synthesis of Intermediates 4.3

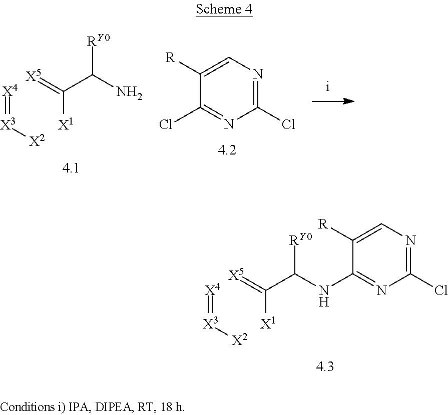

To a solution of 2,4-dichloro-5-substituted pyrimidine 4.2 (1.0 eq) and DIPEA (2.0 eq) in IPA (10 vols) at 0° C. was added drop wise an alpha substituted benzylamine 4.1 (1.0 eq), the resultant mixture was stirred overnight at room temperature. The reaction was either diluted with water and the resultant precipitate of 4.3 collected by filtration, or diluted with EtOAc (20 vols) and water (20 vols) and brine (10 vols). The organic layer was collected, dried (MgSO₄) and concentrated under reduced pressure to give 4.3. Where required, purification was carried out by silica flash chromatography (EtOAc-Petroleum ether gradient).

### Alternative Procedure for the Preparation of Amide Substituted Intermediates 5.4

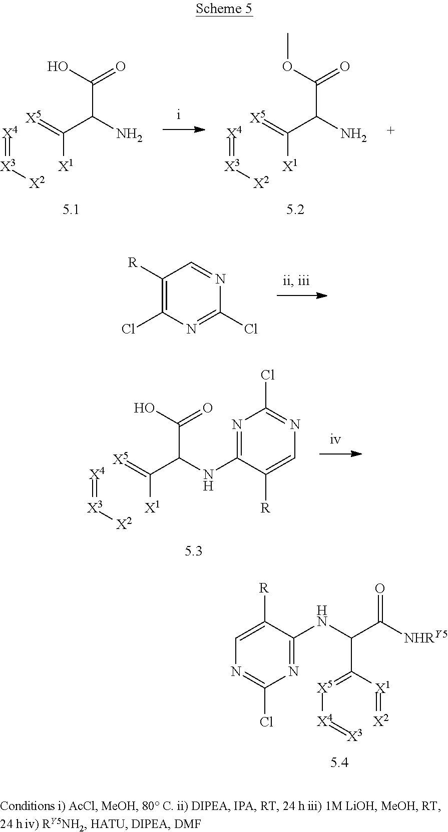

### Step 1

To a stirred solution of an amino acid 5.1 (1.0 eq) in MeOH (2 mL) was added acetyl chloride (2.0 eq). The reaction was stirred for 15 min at room temperature then at 80° C. overnight. The reaction was concentrated in vacuo and the residue was neutralized with sat. NaHCO₃ and extracted into EtOAc to yield the amino ester 5.2

### Step 2

The amino ester 5.2 (1.2 eq) was added to a stirred, cooled (0° C.) solution of 2,4-dichloro-5-substituted pyrimidine (1.0 eq) and DIPEA (2.5 eq) in IPA. The reaction was allowed to warm to room temperature and stirred overnight. The reaction mixture was diluted with water and EtOAc. The organic layer was rinsed (brine), dried (MgSO4) and concentrated in vacuo to give a residue that was used without further purification

### Step 3

The residue from Step 2 was dissolved in MeOH and treated at room temperature with 1 M LiOH (3.0 eq). The reaction mixture was stirred overnight at room temperature then neutralized with 2 M HCl, concentrated and triturated with ether to give the acid 5.3 as an off-white solid.

### Step 4

To a stirred solution of the acid 5.3 (1.0 eq) in DMF (2 mL) was added the relevant amine

R^{Y5}NH2 (1.1 eq), HATU (1.3 eq) and DIPEA (2.0 eq). The reaction was then stirred for 1 h at room temperature. The reaction mixture was concentrated in vacuo. Reverse phase flash chromatography (30 g C18 column, 5%-95% ACN with ammonia in water with ammonia) gave the desired amide 5.4.

### General Synthesis of Test Compounds 6.1

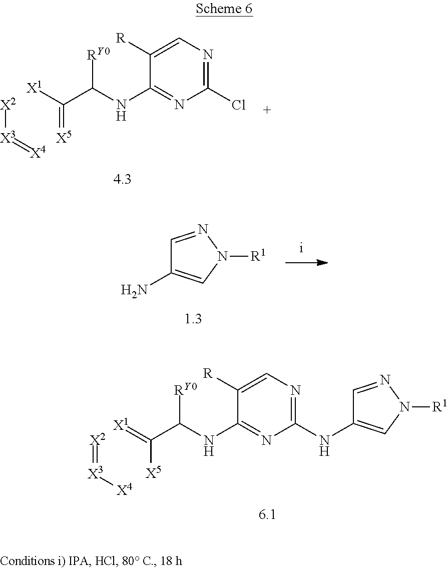

A mixture of the 2-chloropyrimidine intermediate 4.3 (1.0 eq), substituted 1H-pyrazol-4-amine 1.3 (1.0 eq) and 4M HCl in dioxane (0.1 eq) were stirred at 80° C. for 18 h in IPA (5 vols). The reaction was then diluted with EtOAc (20 vols) and NaHCO₃ (10 vols) the organic phase was collected, dried (MgSO₄) and concentrated to yield the desired test compound 6.1, which was further purified by Flash chromatography (EtOAc/Petrol) or HPLC where necessary.

### General Synthesis of Test Compounds 7.2

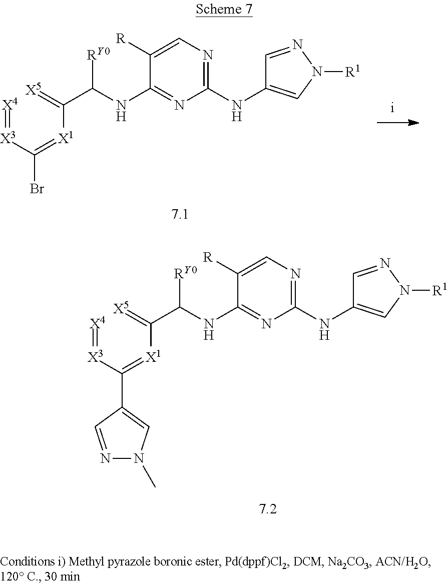

The aryl bromide 7.1 (1.0 eq), 1-methylpyrazole-4-boronic acid, pinacol ester (1.2 eq), 2M Na₂CO₃ (2.0 eq) and Pd(dppf)Cl₂DCM (0.05 eq) in ACN were irradiated in the microwave at 130° C. for 30 minutes. The reaction mixture was diluted with MeOH and filtered. The filtrates were concentrated and purified by prep HPLC to yield 7.2.

### General Synthesis of Test Compounds 8.1

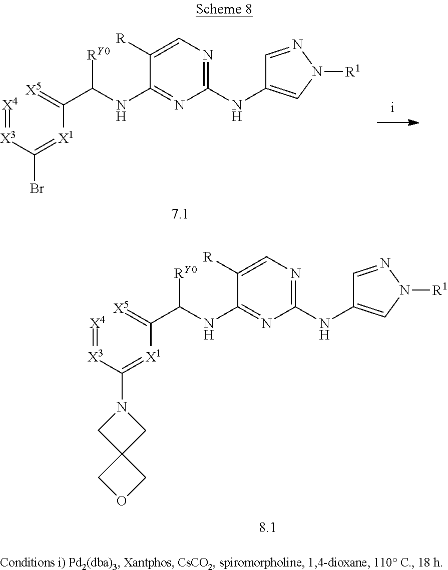

A suspension of the aryl bromide 7.1 (1.0 eq), spiromorpholine (1.5 eq), Pd₂(dba)₃ (0.01 eq), XANTPHOS (0.05 eq) and cesium carbonate (3.0 eq) was refluxed in degassed 1,4-dioxane (10 vols) overnight. The mixture was cooled and filtered. The filtrate was passed though PS-SH cartridge then solvent removed in vacuo. Purification can be achieved by reverse phase chromatography to yield test compound 8.1.

### General Synthesis of Test Compounds 9.2

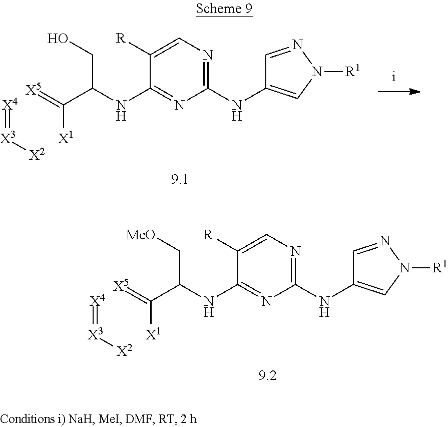

To a solution of compound 9.1 (20 mg) in DMF (2 mL) was added NaH (1.0 eq) followed by MeI (1.5 eq). The reaction mixture was stirred at room temperature for 2 h then quenched with MeOH, concentrated and purified by prep HPLC to yield test compound 9.2.

### General Synthesis of 10.2 and Conversion to Amides 10.3

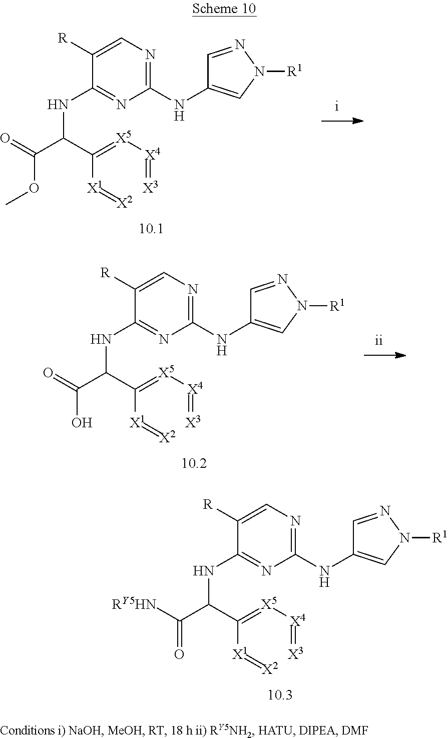

### Step 1

To a solution of compound 10.1 in MeOH was added 1M NaOH (2 eq). The reaction mixture was stirred overnight at room temperature then neutralized with 2 M HCl, concentrated and triturated with ether to give the acid 10.2.

### Step 2

To a stirred solution of 10.2 (1.0 eq) in DMF (2 mL) was added the relevant amine R^{Y5}NH₂ (1.1 eq), HATU (1.3 eq) and DIPEA (2.0 eq). The reaction was then stirred for 1 h at room temperature. The reaction was diluted with DCM, washed (brine), dried (MgSO₄) and concentrated in vacuo. Reverse phase flash chromatography (30 g C18 column, 5%-95% ACN with formic acid in water with formic acid) gave the desired amide 10.3.

### General Synthesis of Test Compounds 11.1

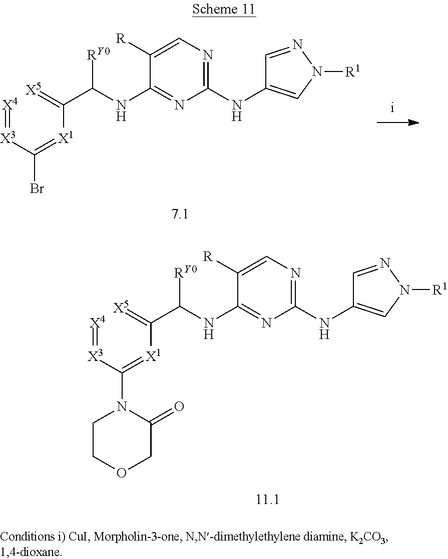

The aryl bromide 7.1 (1.0 eq), morpholin-3-one (1.25 eq), CuI (0.2 eq), N,N′-dimethylethylene diamine (0.4 eq) and K₂CO₃ (2.0 eq) were heated to 110° C. in dioxane for 18 h. The reaction mixture was cooled, diluted with water and EtOAC. The organic layer was rinsed (water, brine), dried (MgSO₄) and concentrated to yield 11.1. Purification was carried out by reverse phase chromatography.

It is clear to a practitioner in the art to combine or adjust such routes, especially in combination with the introduction of activating or protecting chemical groups.

TABLE 5
Benzylamine intermediates

| | | | | LCMS | | |
|---|---|---|---|---|---|---|
| N^{o} | Structure | Formula | MWt | RT | m/z | Method |
| 1 | 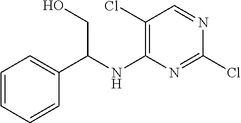 [SMILES: OCC(Nc1nc(Cl)ncc1Cl)c1ccccc1] | C₁₂H₁₁Cl₂N₃O | 284.1 | 2.11 | 284 | C |
| 2 | 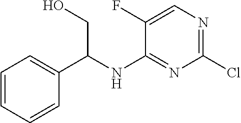 [SMILES: OCC(Nc1nc(Cl)ncc1F)c1ccccc1] | C₁₂H₁₁ClFN₃O | 267.7 | 2.06 | 268 | C |
| 3 | 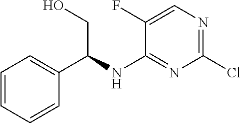 [SMILES: OC[C@@H](Nc1nc(Cl)ncc1F)c1ccccc1] | C₁₂H₁₁ClFN₃O | 267.7 | 2.06 | 268 | C |
| 4 | 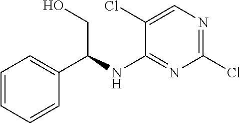 [SMILES: OC[C@@H](Nc1nc(Cl)ncc1Cl)c1ccccc1] | C₁₂H₁₁Cl₂N₃O | 284.1 | 0.98 | 284 | B |
| 5 | 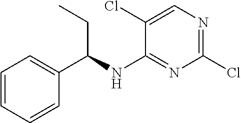 [SMILES: CC[C@@H](Nc1nc(Cl)ncc1Cl)c1ccccc1] | C₁₃H₁₃Cl₂N₃ | 282.2 | 2.95 | 282 | C |
| 6 | 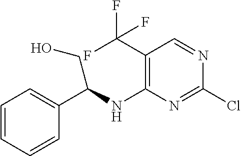 [SMILES: OC[C@@H](Nc1nc(Cl)ncc1C(F)(F)F)c1ccccc1] | C₁₃H₁₁ClF₃N₃O | 317.7 | 2.48 + 2.63 (regio- isomers) | 318 | C |
| 7 | 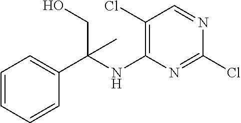 [SMILES: CC(CO)(Nc1nc(Cl)ncc1Cl)c1ccccc1] | C₁₃H₁₃Cl₂N₃O | 298.2 | 2.45 | 298 | C |
| 8 | 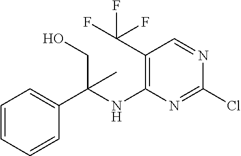 [SMILES: CC(CO)(Nc1nc(Cl)ncc1C(F)(F)F)c1ccccc1] | C₁₄H₁₃ClF₃N₃O | 331.7 | 2.66 + 2.76 (regio- isomers) | 332 | C |
| 9 | 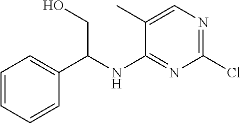 [SMILES: Cc1cnc(Cl)nc1NC(CO)c1ccccc1] | C₁₃H₁₄ClN₃O | 263.7 | 2.06 | 264 | C |
| 10 | 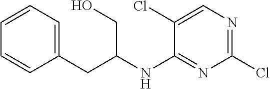 [SMILES: OCC(Cc1ccccc1)Nc1nc(Cl)ncc1Cl] | C₁₃H₁₃Cl₂N₃O | 298.2 | 2.36 | 298 | C |
| 11 | 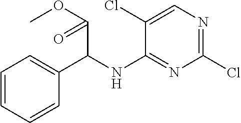 [SMILES: COC(=O)C(Nc1nc(Cl)ncc1Cl)c1ccccc1] | C₁₃H₁₁Cl₂N₃O₂ | 312.2 | 2.68 | 312 | C |
| 12 | 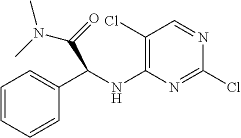 [SMILES: CN(C)C(=O)[C@@H](Nc1nc(Cl)ncc1Cl)c1ccccc1] | C₁₄H₁₄Cl₂N₄O | 325.2 | 2.57 | 325 | C |
| 13 | 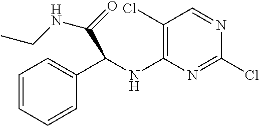 [SMILES: CCNC(=O)[C@@H](Nc1nc(Cl)ncc1Cl)c1ccccc1] | C₁₄H₁₄Cl₂N₄O | 325.2 | 2.47 | 325 | C |
| 14 | 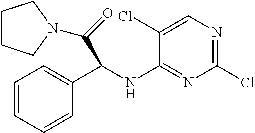 [SMILES: O=C([C@@H](Nc1nc(Cl)ncc1Cl)c1ccccc1)N1CCCC1] | C₁₆H₁₆Cl₂N₄O | 351.2 | 2.66 | 351 | C |
| 15 | 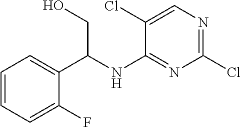 [SMILES: OCC(Nc1nc(Cl)ncc1Cl)c1ccccc1F] | C₁₂H₁₀Cl₂FN₃O | 302.1 | 1.04 | 302 | A |
| 16 | 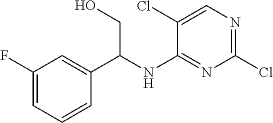 [SMILES: OCC(Nc1nc(Cl)ncc1Cl)c1cccc(F)c1] | C₁₂H₁₀Cl₂FN₃O | 302.1 | 1.04 | 302 | A |
| 17 | 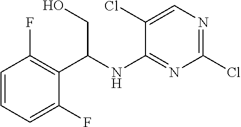 [SMILES: OCC(Nc1nc(Cl)ncc1Cl)c1c(F)cccc1F] | C₁₂H₉Cl₂F₂N₃O | 320.1 | 1.06 | 320 | A |
| 18 | 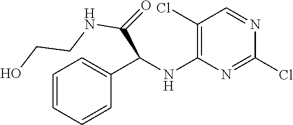 [SMILES: O=C(NCCO)[C@@H](Nc1nc(Cl)ncc1Cl)c1ccccc1] | C₁₄H₁₄Cl₂N₄O₂ | 341.2 | 2.1 | 341 | C |
| 19 | 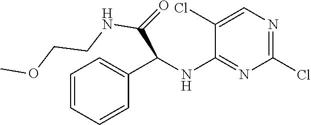 [SMILES: COCCNC(=O)[C@@H](Nc1nc(Cl)ncc1Cl)c1ccccc1] | C₁₅H₁₆Cl₂N₄O₂ | 355.2 | 2.38 | 355 | C |
| 20 | 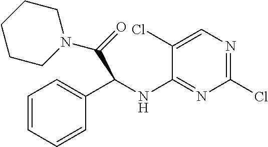 [SMILES: O=C([C@@H](Nc1nc(Cl)ncc1Cl)c1ccccc1)N1CCCCC1] | C₁₇H₁₈Cl₂N₄O | 365.3 | 2.91 | 365 | C |
| 21 | 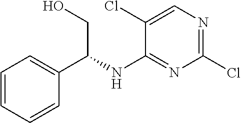 [SMILES: OC[C@H](Nc1nc(Cl)ncc1Cl)c1ccccc1] | C₁₂H₁₁Cl₂N₃O | 284.1 | 0.98 | 284 | B |
| 22 | 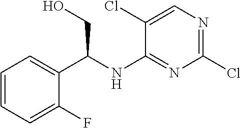 [SMILES: OC[C@@H](Nc1nc(Cl)ncc1Cl)c1ccccc1F] | C₁₂H₁₀Cl₂FN₃O | 302.1 | 1.04 | 302 | B |
| 23 | 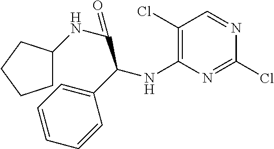 [SMILES: O=C(NC1CCCC1)[C@@H](Nc1nc(Cl)ncc1Cl)c1ccccc1] | C₁₇H₁₈Cl₂N₄O | 365.2 | 2.78 | 365 | C |
| 24 | 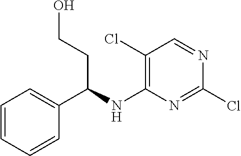 [SMILES: OCC[C@@H](Nc1nc(Cl)ncc1Cl)c1ccccc1] | C₁₃H₁₃Cₗ₂N₃O | 298.2 | 1.05 | 298 | B |
| 25 | 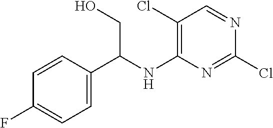 [SMILES: OCC(Nc1nc(Cl)ncc1Cl)c1ccc(F)cc1] | C₁₂H₁₀Cl₂FN₃O | 302.1 | 1.04 | 302 | B |
| 26 | 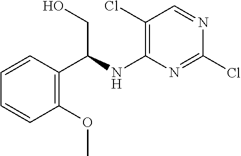 [SMILES: COc1ccccc1[C@@H](CO)Nc1nc(Cl)ncc1Cl] | C₁₃H₁₃Cl₂N₃O₂ | 314.2 | 1.05 | 314 | B |
| 27 | 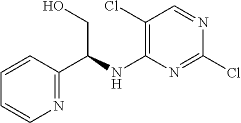 [SMILES: OC[C@@H](Nc1nc(Cl)ncc1Cl)c1ccccn1] | C₁₁H₁₀Cl₂N₄O | 285.1 | 0.91 | 285 | B |
| 28 | 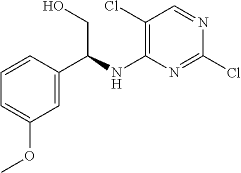 [SMILES: COc1cccc([C@@H](CO)Nc2nc(Cl)ncc2Cl)c1] | C₁₃H₁₃Cl₂N₃O₂ | 314.2 | 1.02 | 314 | B |
| 29 | 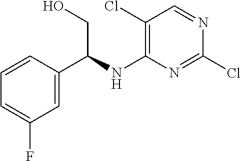 [SMILES: OC[C@@H](Nc1nc(Cl)ncc1Cl)c1cccc(F)c1] | C₁₂H₁₀Cl₂FN₃O | 302.1 | 1.04 | 302 | B |
| 30 | 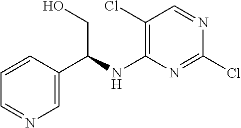 [SMILES: OC[C@@H](Nc1nc(Cl)ncc1Cl)c1cccnc1] | C₁₁H₁₀Cl₂N₄O | 285.1 | 0.84 | 285 | B |
| 31 | 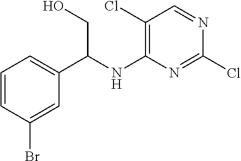 [SMILES: OCC(Nc1nc(Cl)ncc1Cl)c1cccc(Br)c1] | C₁₂H₁₀BrCl₂N₃O | 363.0 | 1.11 | 362 | B |
| 32 | 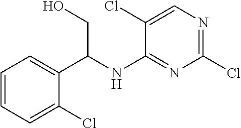 [SMILES: OCC(Nc1nc(Cl)ncc1Cl)c1ccccc1Cl] | C₁₂H₁₀Cl₃N₃O | 318.6 | 1.04 | 318 | B |
| 33 | 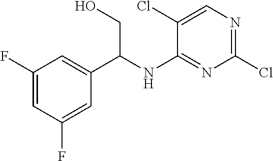 [SMILES: OCC(Nc1nc(Cl)ncc1Cl)c1cc(F)cc(F)c1] | C₁₂H₉Cl₂F₂N₃O | 320.1 | 1.05 | 320 | B |
| 34 | 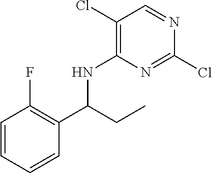 [SMILES: CCC(Nc1nc(Cl)ncc1Cl)c1ccccc1F] | C₁₃H₁₂Cl₂FN₃ | 300.2 | 1.23 | 300 | B |
| 35 | 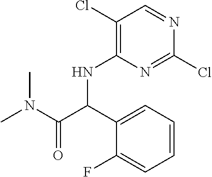 [SMILES: CN(C)C(=O)C(Nc1nc(Cl)ncc1Cl)c1ccccc1F] | C₁₄H₁₃Cl₂FN₄O | 343.2 | 1.03 | 344 | B |
| 36 | 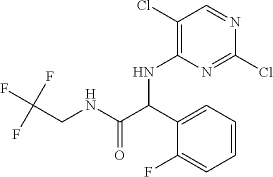 [SMILES: O=C(NCC(F)(F)F)C(Nc1nc(Cl)ncc1Cl)c1ccccc1F] | C₁₄H₁₀Cl₂F₄N₄O | 397.2 | 1.07 | 397 | B |
| 37 | 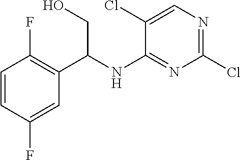 [SMILES: OCC(Nc1nc(Cl)ncc1Cl)c1cc(F)ccc1F] | C₁₆H₁₅ClF₂N₆O | 380.8 | 1.03 | 320 | B |
| 38 | 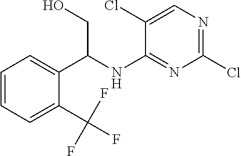 [SMILES: OCC(Nc1nc(Cl)ncc1Cl)c1ccccc1C(F)(F)F] | C₁₃H₁₀Cl₂F₃N₃O | 352.1 | 1.08 | 352 | B |
| 39 | 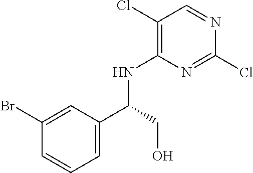 [SMILES: OC[C@@H](Nc1nc(Cl)ncc1Cl)c1cccc(Br)c1] | C₁₂H₁₀BrCl₂N₃O | 363.0 | 1.08 | 364 | B |
| 40 | 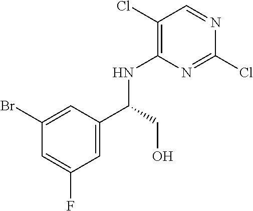 [SMILES: OC[C@@H](Nc1nc(Cl)ncc1Cl)c1cc(F)cc(Br)c1] | C₁₂H₉BrCl₂FN₃O | 381.0 | 1.11 | 382 | B |
| 41 | 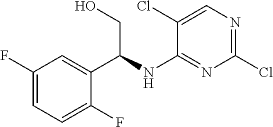 [SMILES: OC[C@@H](Nc1nc(Cl)ncc1Cl)c1cc(F)ccc1F] | C₁₂H₉Cl₂F₂N₃O | 320.1 | 1.03 | 320 | B |
| 42 | 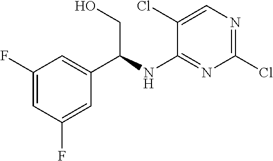 [SMILES: OC[C@@H](Nc1nc(Cl)ncc1Cl)c1cc(F)cc(F)c1] | C₁₂H₉Cl₂F₂N₃O | 320.1 | 1.05 | 320 | B |
| 43 | 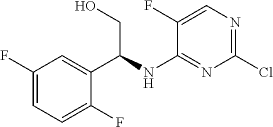 [SMILES: OC[C@@H](Nc1nc(Cl)ncc1F)c1cc(F)ccc1F] | C₁₂H₉ClF₃N₃O | 303.7 | 0.98 | 304 | B |
| 44 | 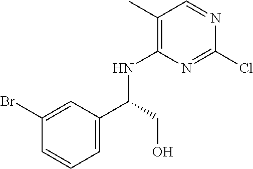 [SMILES: Cc1cnc(Cl)nc1N[C@H](CO)c1cccc(Br)c1] | C₁₃H₁₃BrClN₃O | 342.6 | 1.02 | 344 | B |
| 45 | 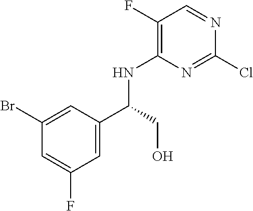 [SMILES: OC[C@@H](Nc1nc(Cl)ncc1F)c1cc(F)cc(Br)c1] | C₁₂H₉BrClF₂N₃O | 364.6 | 1.06 | 366 | B |
| 46 | 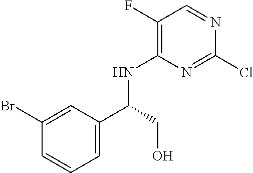 [SMILES: OC[C@@H](Nc1nc(Cl)ncc1F)c1cccc(Br)c1] | C₁₂H₁₀BrClFN₃O | 346.6 | 1.03 | 348 | B |
| 47 | 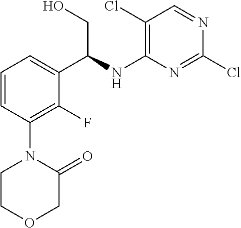 [SMILES: O=C1COCCN1c1cccc([C@@H](CO)Nc2nc(Cl)ncc2Cl)c1F] | C₁₆H₁₅Cl₂FN₄O₃ | 401.2 | 0.87 | 401 | B |
| 48 | 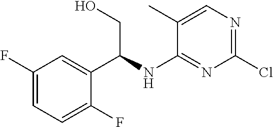 [SMILES: Cc1cnc(Cl)nc1N[C@H](CO)c1cc(F)ccc1F] | C₁₃H₁₂ClF₂N₃O | 299.7 | 0.87 | 401 | B |
| 49 | 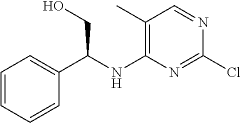 [SMILES: Cc1cnc(Cl)nc1N[C@H](CO)c1ccccc1] | C₁₃H₁₄ClN₃O | 263.7 | 2.14 | 264 | D |
| 50 | 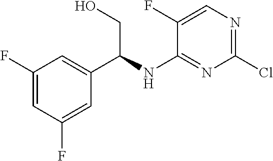 [SMILES: OC[C@@H](Nc1nc(Cl)ncc1F)c1cc(F)cc(F)c1] | C₁₂H₉ClF₃N₃O | 303.7 | 0.99 | 304 | B |
| 51 | 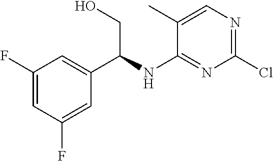 [SMILES: Cc1cnc(Cl)nc1N[C@H](CO)c1cc(F)cc(F)c1] | C₁₃H₁₂ClF₂N₃O | 299.7 | 0.98 | 300 | B |
| 52 | 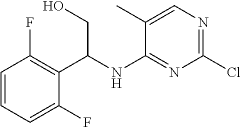 [SMILES: Cc1cnc(Cl)nc1NC(CO)c1c(F)cccc1F] | C₁₃H₁₂ClF₂N₃O | 299.7 | 0.96 | 300 | B |

TABLE 6
Test compounds

| | | | | LCMS | | |
|---|---|---|---|---|---|---|
| N^{o} | Structure | Formula | MWt | RT | m/z | Method |
| 1 | 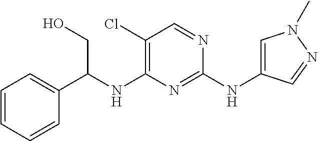 [SMILES: Cn1cc(Nc2ncc(Cl)c(NC(CO)c3ccccc3)n2)cn1] | C₁₆H₁₇ClN₆O | 344.8 | 1.66 | 345 | C |
| 2 | 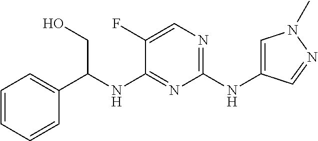 [SMILES: Cn1cc(Nc2ncc(F)c(NC(CO)c3ccccc3)n2)cn1] | C₁₆H₁₇FN₆O | 328.3 | 1.4 | 329 | C |
| 3 | 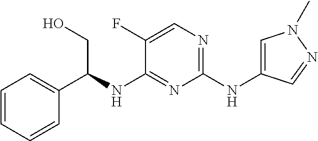 [SMILES: Cn1cc(Nc2ncc(F)c(N[C@H](CO)c3ccccc3)n2)cn1] | C₁₆H₁₇FN₆O | 328.3 | 1.41 | 329 | C |
| 4 | 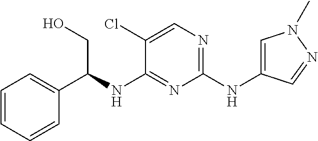 [SMILES: Cn1cc(Nc2ncc(Cl)c(N[C@H](CO)c3ccccc3)n2)cn1] | C₁₆H₁₇ClN₆O | 344.8 | 1.59 | 345 | C |
| 5 | 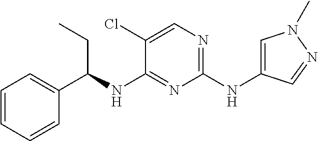 [SMILES: CC[C@@H](Nc1nc(Nc2cnn(C)c2)ncc1Cl)c1ccccc1] | C₁₇H₁₉ClN₆ | 342.8 | 1.97 | 343 | C |
| 6 | 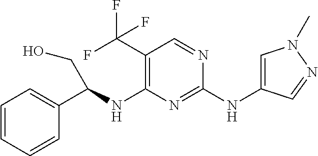 [SMILES: Cn1cc(Nc2ncc(C(F)(F)F)c(N[C@H](CO)c3ccccc3)n2)cn1] | C₁₇H₁₇F₃N₆O | 378.3 | 1.97 | 379 | C |
| 7 | 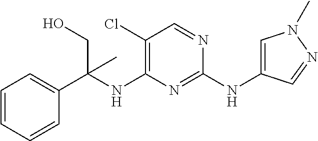 [SMILES: Cn1cc(Nc2ncc(Cl)c(NC(C)(CO)c3ccccc3)n2)cn1] | C₁₇H₁₉ClN₆O | 358.8 | 2.21 | 359 | D |
| 8 | 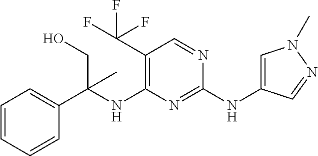 [SMILES: Cn1cc(Nc2ncc(C(F)(F)F)c(NC(C)(CO)c3ccccc3)n2)cn1] | C₁₈H₁₉F₃N₆O | 392.4 | 2.41 | 393 | D |
| 9 | 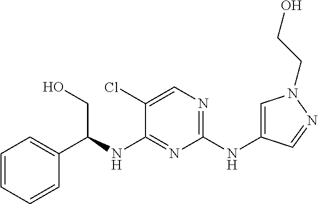 [SMILES: OCCn1cc(Nc2ncc(Cl)c(N[C@H](CO)c3ccccc3)n2)cn1] | C₁₇H₁₉ClN₆O₂ | 374.8 | 1.49 | 375 | D |
| 10 | 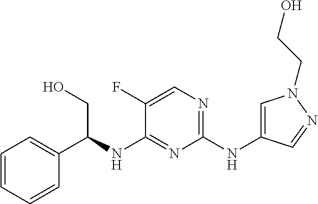 [SMILES: OCCn1cc(Nc2ncc(F)c(N[C@H](CO)c3ccccc3)n2)cn1] | C₁₇H₁₉FN₆O₂ | 358.4 | 1.37 | 359 | C |
| 11 | 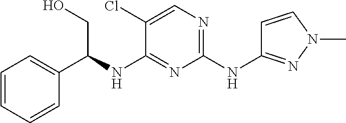 [SMILES: Cn1ccc(Nc2ncc(Cl)c(N[C@H](CO)c3ccccc3)n2)n1] | C₁₆H₁₇ClN₆O | 344.8 | 0.96 | 345 | B |
| 12 | 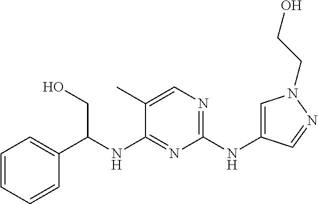 [SMILES: Cc1cnc(Nc2cnn(CCO)c2)nc1NC(CO)c1ccccc1] | C₁₈H₂₂N₆O₂ | 354.4 | 1.41 | 355 | C |
| 13 | 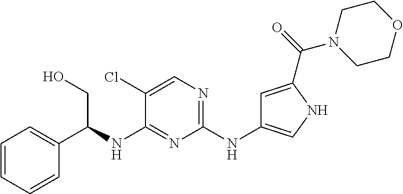 [SMILES: O=C(c1cc(Nc2ncc(Cl)c(N[C@H](CO)c3ccccc3)n2)c[nH]1)N1CCOCC1] | C₂₁H₂₃ClN₆O₃ | 442.9 | 0.92 | 443 | B |
| 14 | 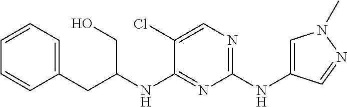 [SMILES: Cn1cc(Nc2ncc(Cl)c(NC(CO)Cc3ccccc3)n2)cn1] | C₁₇H₁₉ClN₆O | 358.8 | 1.65 | 359 | C |
| 15 | 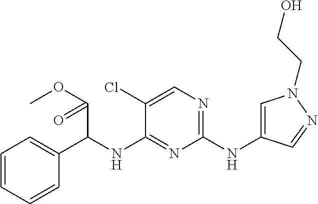 [SMILES: COC(=O)C(Nc1nc(Nc2cnn(CCO)c2)ncc1Cl)c1ccccc1] | C₁₈H₁₉ClN₆O₃ | 402.8 | 0.89 | 403 | A |
| 16 | 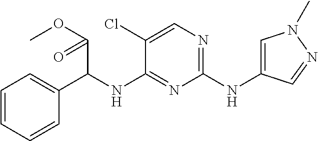 [SMILES: COC(=O)C(Nc1nc(Nc2cnn(C)c2)ncc1Cl)c1ccccc1] | C₁₇H₁₇ClN₆O₂ | 372.8 | 2.06 | 373 | C |
| 17 | 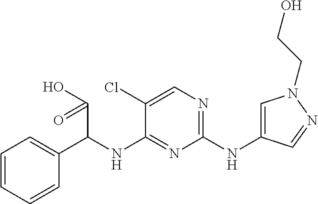 [SMILES: O=C(O)C(Nc1nc(Nc2cnn(CCO)c2)ncc1Cl)c1ccccc1] | C₁₇H₁₇ClN₆O₃ | 388.8 | 1.63 | 389 | C |
| 18 | 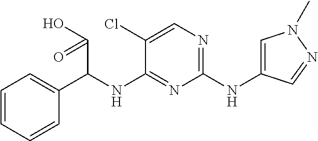 [SMILES: Cn1cc(Nc2ncc(Cl)c(NC(C(=O)O)c3ccccc3)n2)cn1] | C₁₆H₁₅ClN₆O₂ | 358.8 | 1.72 | 359 | C |
| 19 | 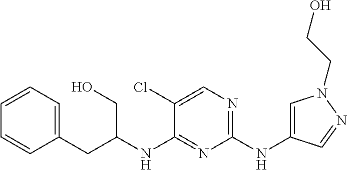 [SMILES: OCCn1cc(Nc2ncc(Cl)c(NC(CO)Cc3ccccc3)n2)cn1] | C₁₈H₂₁ClN₆O₂ | 388.9 | 1.54 | 389 | C |
| 20 | 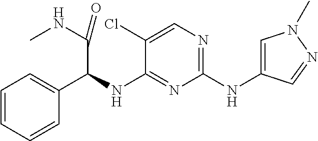 [SMILES: CNC(=O)[C@@H](Nc1nc(Nc2cnn(C)c2)ncc1Cl)c1ccccc1] | C₁₇H₁₈ClN₇O | 371.8 | 0.83 | 372 | A |
| 21 | 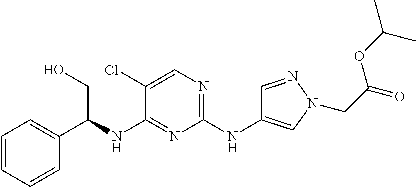 [SMILES: CC(C)OC(=O)Cn1cc(Nc2ncc(Cl)c(N[C@H](CO)c3ccccc3)n2)cn1] | C₂₀H₂₃ClN₆O₃ | 430.9 | 1.02 | 431 | B |
| 22 | 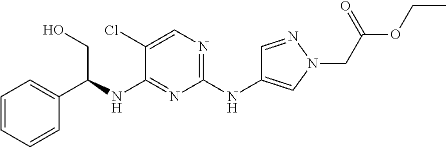 [SMILES: CCOC(=O)Cn1cc(Nc2ncc(Cl)c(N[C@H](CO)c3ccccc3)n2)cn1] | C₁₉H₂₁ClN₆O₃ | 416.9 | 0.98 | 417 | B |
| 23 | 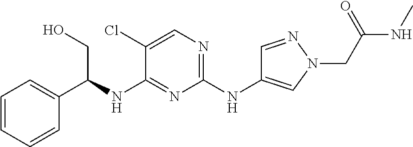 [SMILES: CNC(=O)Cn1cc(Nc2ncc(Cl)c(N[C@H](CO)c3ccccc3)n2)cn1] | C₁₈H₂₀ClN₇O₂ | 401.9 | 0.86 | 402 | B |
| 24 | 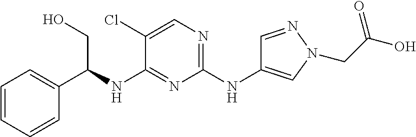 [SMILES: O=C(O)Cn1cc(Nc2ncc(Cl)c(N[C@H](CO)c3ccccc3)n2)cn1] | C₁₇H₁₇ClN₆O₃ | 388.8 | 0.74 | 389 | B |
| 25 | 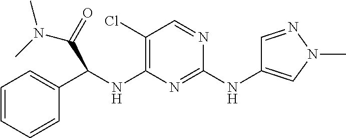 [SMILES: CN(C)C(=O)[C@@H](Nc1nc(Nc2cnn(C)c2)ncc1Cl)c1ccccc1] | C₁₈H₂₀ClN₇O | 385.9 | 5.94 | 386 | E |
| 26 | 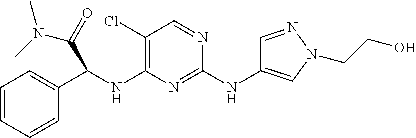 [SMILES: CN(C)C(=O)[C@@H](Nc1nc(Nc2cnn(CCO)c2)ncc1Cl)c1ccccc1] | C₁₉H₂₂ClN₇O₂ | 415.9 | 5.57 | 416 | E |
| 27 | 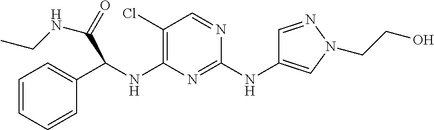 [SMILES: CCNC(=O)[C@@H](Nc1nc(Nc2cnn(CCO)c2)ncc1Cl)c1ccccc1] | C₁₉H₂₂ClN₇O₂ | 415.9 | 5.81 | 416 | E |
| 28 | 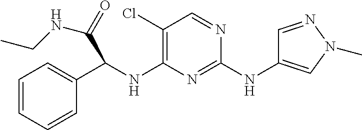 [SMILES: CCNC(=O)[C@@H](Nc1nc(Nc2cnn(C)c2)ncc1Cl)c1ccccc1] | C₁₈H₂₀ClN₇O | 385.9 | 6.34 | 386 | E |
| 29 | 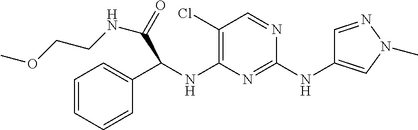 | C₁₉H₂₂ClN₇O₂ | 415.9 | 6.14 | 416 | E |
| 30 | 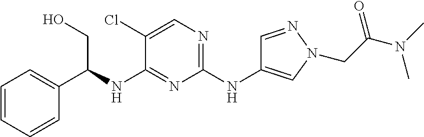 [SMILES: CN(C)C(=O)Cn1cc(Nc2ncc(Cl)c(N[C@H](CO)c3ccccc3)n2)cn1] | C₁₉H₂₂ClN₇O₂ | 415.9 | 0.89 | 416 | B |
| 31 | 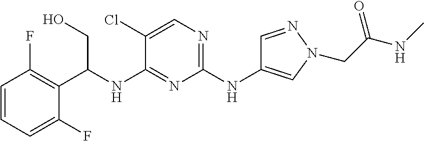 [SMILES: CNC(=O)Cn1cc(Nc2ncc(Cl)c(NC(CO)c3c(F)cccc3F)n2)cn1] | C₁₈H₁₈ClF₂N₇O₂ | 437.8 | 0.9 | 438 | B |
| 32 | 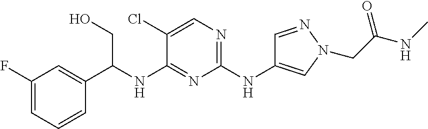 [SMILES: CNC(=O)Cn1cc(Nc2ncc(Cl)c(NC(CO)c3cccc(F)c3)n2)cn1] | C₁₈H₁₉ClFN₇O₂ | 419.8 | 0.88 | 420 | B |
| 33 | 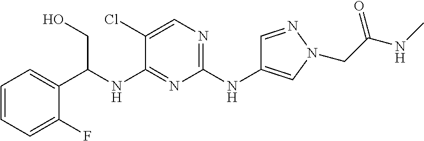 [SMILES: CNC(=O)Cn1cc(Nc2ncc(Cl)c(NC(CO)c3ccccc3F)n2)cn1] | C₁₈H₁₉ClFN₇O₂ | 419.8 | 0.87 | 420 | B |
| 34 | 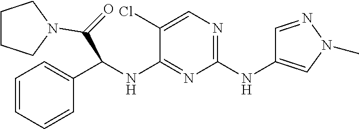 [SMILES: Cn1cc(Nc2ncc(Cl)c(N[C@H](C(=O)N3CCCC3)c3ccccc3)n2)cn1] | C₂₀H₂₂ClN₇O | 411.9 | 6.44 | 412 | E |
| 35 | 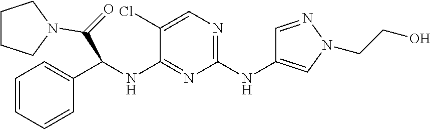 [SMILES: O=C([C@@H](Nc1nc(Nc2cnn(CCO)c2)ncc1Cl)c1ccccc1)N1CCCC1] | C₂₁H₂₄ClN₇O₂ | 441.9 | 5.89 | 442 | E |
| 36 | 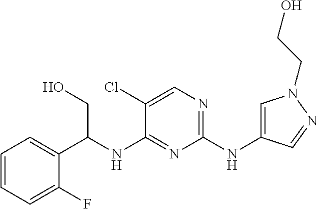 [SMILES: OCCn1cc(Nc2ncc(Cl)c(NC(CO)c3ccccc3F)n2)cn1] | C₁₇H₁₈ClFN₆O₂ | 392.8 | 4.93 | 393 | E |
| 37 | 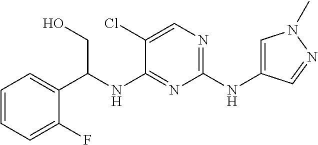 [SMILES: Cn1cc(Nc2ncc(Cl)c(NC(CO)c3ccccc3F)n2)cn1] | C₁₆H₁₆ClFN₆O | 362.8 | 5.38 | 363 | E |
| 38 | 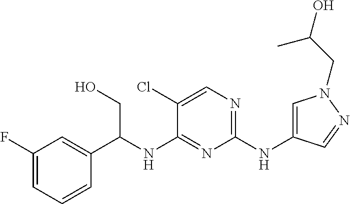 [SMILES: CC(O)Cn1cc(Nc2ncc(Cl)c(NC(CO)c3cccc(F)c3)n2)cn1] | C₁₈H₂₀ClFN₆O₂ | 406.8 | 5.28 | 407 | E |
| 39 | 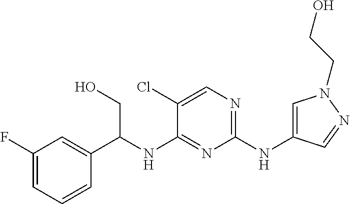 [SMILES: OCCn1cc(Nc2ncc(Cl)c(NC(CO)c3cccc(F)c3)n2)cn1] | C₁₇H₁₈ClFN₆O₂ | 392.8 | 5.02 | 393 | E |
| 40 | 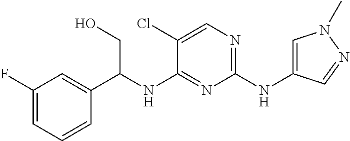 [SMILES: Cn1cc(Nc2ncc(Cl)c(NC(CO)c3cccc(F)c3)n2)cn1] | C₁₆H₁₆ClFN₆O | 362.8 | 5.42 | 363 | E |
| 41 | 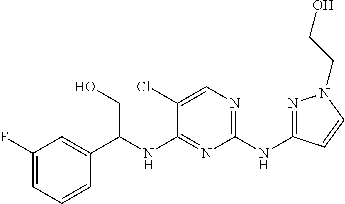 [SMILES: OCCn1ccc(Nc2ncc(Cl)c(NC(CO)c3cccc(F)c3)n2)n1] | C₁₇H₁₈ClFN₆O₂ | 392.8 | 5.2 | 393 | E |
| 42 | 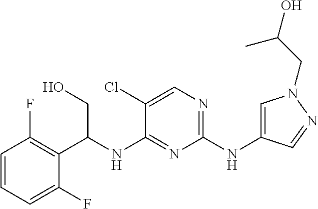 [SMILES: CC(O)Cn1cc(Nc2ncc(Cl)c(NC(CO)c3c(F)cccc3F)n2)cn1] | C₁₈H₁₉ClF₂N₆O₂ | 424.8 | 5.52 | 425 | E |
| 43 | 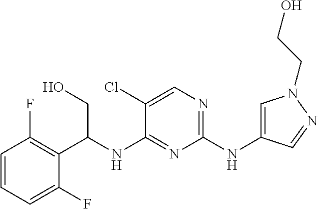 [SMILES: OCCn1cc(Nc2ncc(Cl)c(NC(CO)c3c(F)cccc3F)n2)cn1] | C₁₇H₁₇ClF₂N₆O₂ | 410.8 | 5.23 | 411 | E |
| 44 | 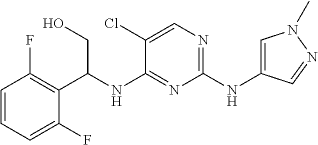 [SMILES: Cn1cc(Nc2ncc(Cl)c(NC(CO)c3c(F)cccc3F)n2)cn1] | C₁₆H₁₅ClF₂N₆O | 380.8 | 5.64 | 381 | E |
| 45 | 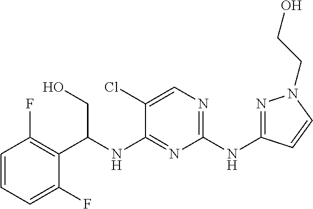 [SMILES: OCCn1ccc(Nc2ncc(Cl)c(NC(CO)c3c(F)cccc3F)n2)n1] | C₁₇H₁₇ClF₂N₆O₂ | 410.8 | 5.28 | 411 | E |
| 46 | 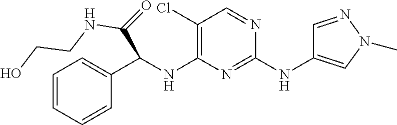 [SMILES: Cn1cc(Nc2ncc(Cl)c(N[C@H](C(=O)NCCO)c3ccccc3)n2)cn1] | C₁₈H₂₀ClN₇O₂ | 401.8 | 5.33 | 402 | E |
| 47 | 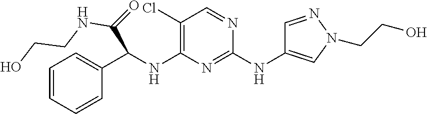 [SMILES: O=C(NCCO)[C@@H](Nc1nc(Nc2cnn(CCO)c2)ncc1Cl)c1ccccc1] | C₁₉H₂₂ClN₇O₃ | 431.9 | 5.05 | 432 | E |
| 48 | 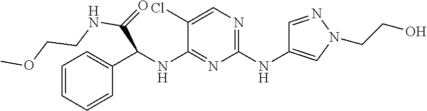 [SMILES: COCCNC(=O)[C@@H](Nc1nc(Nc2cnn(CCO)c2)ncc1Cl)c1ccccc1] | C₂₀H₂₄ClN₇O₃ | 445.9 | 5.59 | 446 | E |
| 49 | 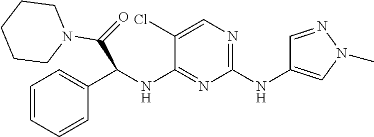 [SMILES: Cn1cc(Nc2ncc(Cl)c(N[C@H](C(=O)N3CCCCC3)c3ccccc3)n2)cn1] | C₂₁H₂₄ClN₇O | 425.9 | 7.21 | 426 | E |
| 50 | 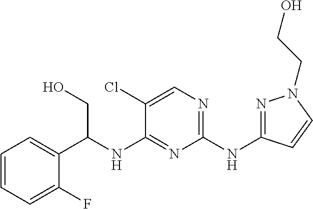 [SMILES: OCCn1ccc(Nc2ncc(Cl)c(NC(CO)c3ccccc3F)n2)n1] | C₁₇H₁₈ClFN₆O₂ | 392.8 | 4.93 | 393 | E |
| 51 | 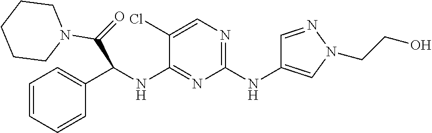 [SMILES: O=C([C@@H](Nc1nc(Nc2cnn(CCO)c2)ncc1Cl)c1ccccc1)N1CCCCC1] | C₁₇H₁₇ClF₂N₆O₈ | 455.9 | 6.54 | 456 | E |
| 52 | 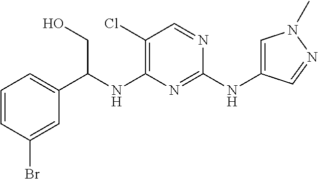 [SMILES: Cn1cc(Nc2ncc(Cl)c(NC(CO)c3cccc(Br)c3)n2)cn1] | C₁₆H₁₆BrClN₆O | 423.7 | 0.85 | 423 | A |
| 53 | 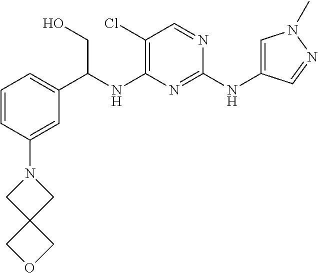 [SMILES: Cn1cc(Nc2ncc(Cl)c(NC(CO)c3cccc(N4CC5(COC5)C4)c3)n2)cn1] | C₂₁H₂₄ClN₇O₂ | 441.9 | 5.01 | 442 | E |
| 54 | 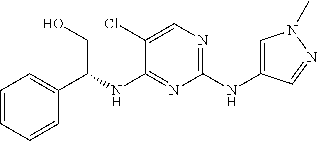 [SMILES: Cn1cc(Nc2ncc(Cl)c(N[C@@H](CO)c3ccccc3)n2)cn1] | C₁₆H₁₇ClN₆O | 344.8 | 1.59 | 345 | C |
| 55 | 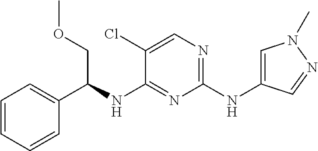 [SMILES: COC[C@@H](Nc1nc(Nc2cnn(C)c2)ncc1Cl)c1ccccc1] | C17H19ClN6O | 358.8 | 2.49 | 359 | D |
| 56 | 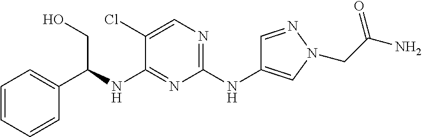 [SMILES: NC(=O)Cn1cc(Nc2ncc(Cl)c(N[C@H](CO)c3ccccc3)n2)cn1] | C₁₇H₁₈ClN₇O₂ | 387.8 | 0.84 | 388 | B |
| 57 | 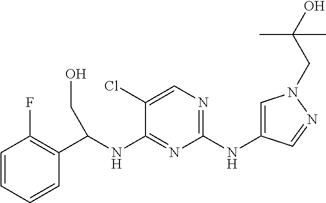 [SMILES: CC(C)(O)Cn1cc(Nc2ncc(Cl)c(NC(CO)c3ccccc3F)n2)cn1] | C₁₉H₂₂ClFN₆O₂ | 420.9 | 5.38 | 421 | E |
| 58 | 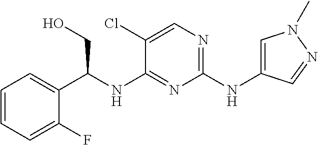 [SMILES: Cn1cc(Nc2ncc(Cl)c(N[C@H](CO)c3ccccc3F)n2)cn1] | C₁₆H₁₆ClFN₆O | 362.8 | 5.43 | 363 | E |
| 59 | 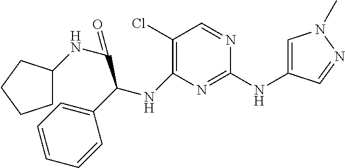 [SMILES: Cn1cc(Nc2ncc(Cl)c(N[C@H](C(=O)NC3CCCC3)c3ccccc3)n2)cn1] | C₂₁H₂₄ClN₇O | 425.9 | 7.8 | 426 | E |
| 60 | 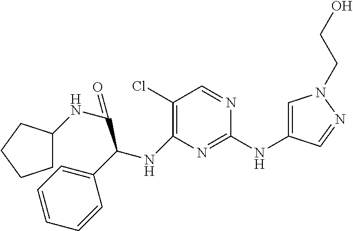 [SMILES: O=C(NC1CCCC1)[C@@H](Nc1nc(Nc2cnn(CCO)c2)ncc1Cl)c1ccccc1] | C₂₂H₂₆ClN₇O₂ | 455.9 | 6.98 | 456 | E |
| 61 | 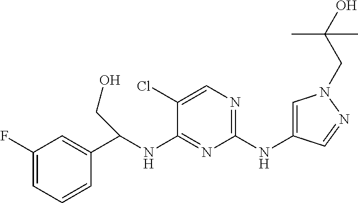 [SMILES: CC(C)(O)Cn1cc(Nc2ncc(Cl)c(NC(CO)c3cccc(F)c3)n2)cn1] | C₁₉H₂₂ClFN₆O₂ | 420.9 | 5.67 | 421 | E |
| 62 | 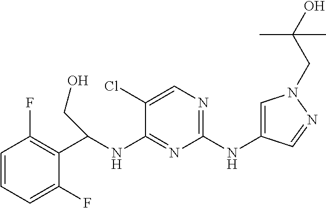 [SMILES: CC(C)(O)Cn1cc(Nc2ncc(Cl)c(NC(CO)c3c(F)cccc3F)n2)cn1] | C₁₉H₂₁ClF₂N₆O₂ | 438.9 | 5.86 | 439 | E |
| 63 | 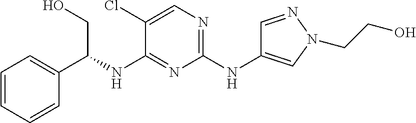 [SMILES: OCCn1cc(Nc2ncc(Cl)c(N[C@@H](CO)c3ccccc3)n2)cn1] | C₁₇H₁₉ClN₆O₂ | 374.8 | 0.94 | 375 | B |
| 64 | 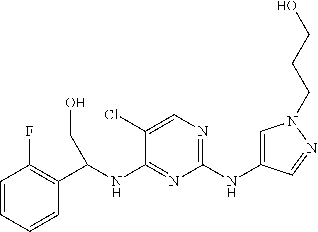 [SMILES: OCCCn1cc(Nc2ncc(Cl)c(NC(CO)c3ccccc3F)n2)cn1] | C₁₈H₂₀ClFN₆O₂ | 406.8 | 5.21 | 407 | E |
| 65 | 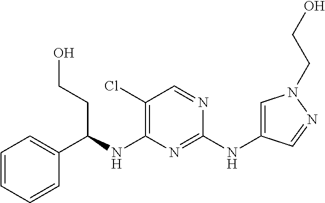 [SMILES: OCC[C@@H](Nc1nc(Nc2cnn(CCO)c2)ncc1Cl)c1ccccc1] | C₁₈H₂₁ClN₆O₂ | 388.9 | 4.91 | 389 | E |
| 66 | 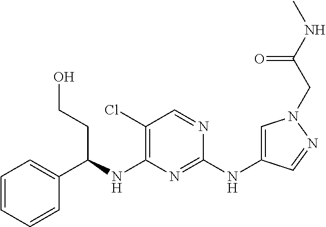 [SMILES: CNC(=O)Cn1cc(Nc2ncc(Cl)c(N[C@H](CCO)c3ccccc3)n2)cn1] | C₁₉H₂₂ClN₇O₂ | 415.9 | 4.96 | 416 | E |
| 67 | 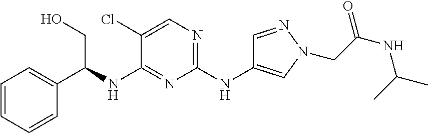 [SMILES: CC(C)NC(=O)Cn1cc(Nc2ncc(Cl)c(N[C@H](CO)c3ccccc3)n2)cn1] | C₂₀H₂₄ClN₇O₂ | 429.9 | 0.94 | 430 | B |
| 68 | 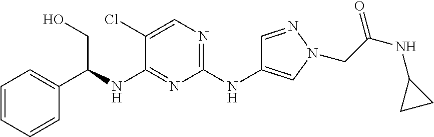 [SMILES: O=C(Cn1cc(Nc2ncc(Cl)c(N[C@H](CO)c3ccccc3)n2)cn1)NC1CC1] | C₂₀H₂₂ClN₇O₂ | 427.9 | 0.91 | 428 | B |
| 69 | 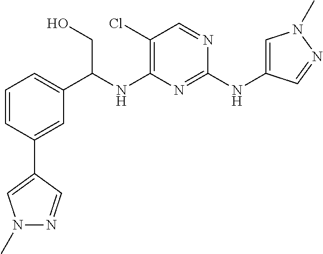 [SMILES: Cn1cc(Nc2ncc(Cl)c(NC(CO)c3cccc(-c4cnn(C)c4)c3)n2)cn1] | C₂₀H₂₁ClN₈O | 424.9 | 5.17 | 425 | E |
| 70 | 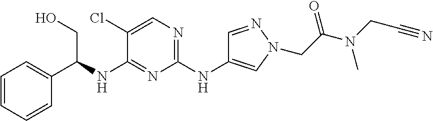 [SMILES: CN(CC#N)C(=O)Cn1cc(Nc2ncc(Cl)c(N[C@H](CO)c3ccccc3)n2)cn1] | C₂₀H₂₁ClN₈O₂ | 440.9 | 0.91 | 441 | B |
| 71 | 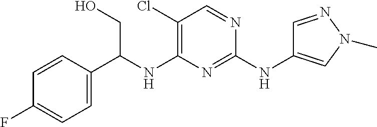 [SMILES: Cn1cc(Nc2ncc(Cl)c(NC(CO)c3ccc(F)cc3)n2)cn1] | C₁₆H₁₆ClFN₆O | 362.8 | 5.83 | 363 | E |
| 72 | 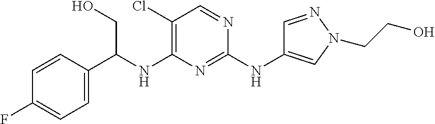 [SMILES: OCCn1cc(Nc2ncc(Cl)c(NC(CO)c3ccc(F)cc3)n2)cn1] | C₁₇H₁₈ClFN₆O₂ | 392.8 | 5.07 | 393 | E |
| 73 | 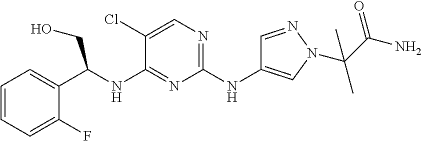 [SMILES: CC(C)(C(N)=O)n1cc(Nc2ncc(Cl)c(N[C@H](CO)c3ccccc3F)n2)cn1] | C₁₉H₂₁ClFN₇O₂ | 433.9 | 5.41 | 434 | E |
| 74 | 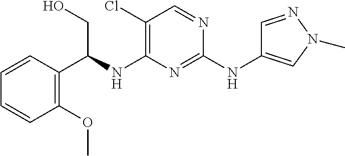 [SMILES: COc1ccccc1[C@@H](CO)Nc1nc(Nc2cnn(C)c2)ncc1Cl] | C₁₇H₁₉ClN₆O₂ | 374.8 | 5.49 | 375 | E |
| 75 | 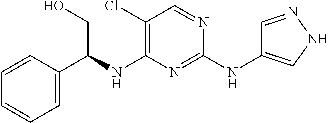 [SMILES: OC[C@@H](Nc1nc(Nc2cn[nH]c2)ncc1Cl)c1ccccc1] | C₁₅H₁₅ClN₆O | 330.8 | 4.7 | 331 | E |
| 76 | 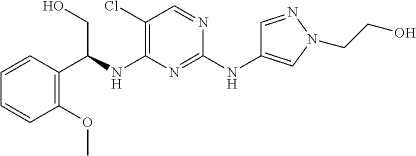 [SMILES: COc1ccccc1[C@@H](CO)Nc1nc(Nc2cnn(CCO)c2)ncc1Cl] | C₁₈H₂₁ClN₆O₃ | 404.9 | 5.16 | 405 | E |
| 77 | 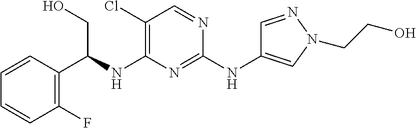 [SMILES: OCCn1cc(Nc2ncc(Cl)c(N[C@H](CO)c3ccccc3F)n2)cn1] | C₁₇H₁₈ClFN₆O₂ | 392.8 | 5 | 393 | E |
| 78 | 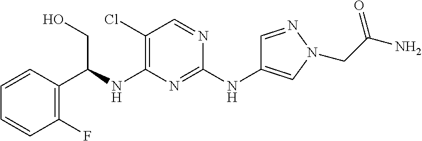 [SMILES: NC(=O)Cn1cc(Nc2ncc(Cl)c(N[C@H](CO)c3ccccc3F)n2)cn1] | C₁₇H₁₇ClFN₇O₂ | 405.8 | 4.86 | 406 | E |
| 79 | 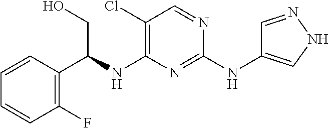 [SMILES: OC[C@@H](Nc1nc(Nc2cn[nH]c2)ncc1Cl)c1ccccc1F] | C₁₅H₁₄ClFN₆O | 348.8 | 4.9 | 349 | E |
| 80 | 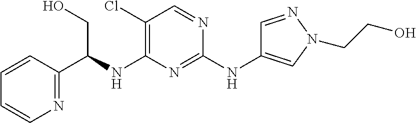 [SMILES: OCCn1cc(Nc2ncc(Cl)c(N[C@H](CO)c3ccccn3)n2)cn1] | C₁₆H₁₈ClN₇O₂ | 375.8 | 3.97 | 376 | E |
| 81 | 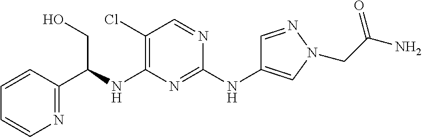 [SMILES: NC(=O)Cn1cc(Nc2ncc(Cl)c(N[C@H](CO)c3ccccn3)n2)cn1] | C₁₆H₁₇ClN₈O₂ | 388.8 | 3.78 | 389 | E |
| 82 | 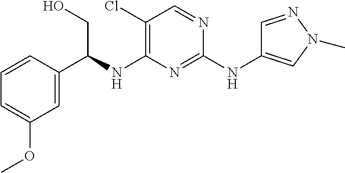 [SMILES: COc1cccc([C@@H](CO)Nc2nc(Nc3cnn(C)c3)ncc2Cl)c1] | C₁₇H₁₉ClN₆O₂ | 374.8 | 5.26 | 375 | E |
| 83 | 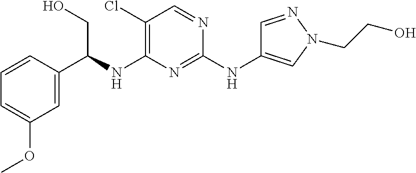 [SMILES: COc1cccc([C@@H](CO)Nc2nc(Nc3cnn(CCO)c3)ncc2Cl)c1] | C₁₈H₂₁ClN₆O₃ | 404.9 | 5.01 | 405 | E |
| 84 | 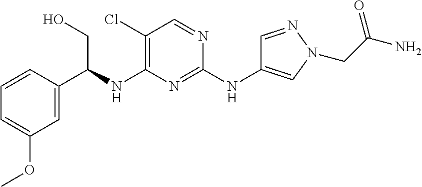 [SMILES: COc1cccc([C@@H](CO)Nc2nc(Nc3cnn(CC(N)=O)c3)ncc2Cl)c1] | C₁₈H₂₀ClN₇O₃ | 417.8 | 4.83 | 418 | E |
| 85 | 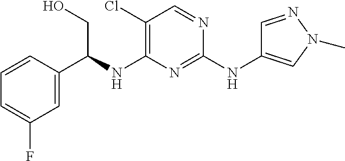 [SMILES: Cn1cc(Nc2ncc(Cl)c(N[C@H](CO)c3cccc(F)c3)n2)cn1] | C₁₆H₁₆ClFN₆O | 362.8 | 5.51 | 363 | E |
| 86 | 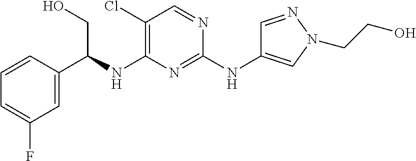 [SMILES: OCCn1cc(Nc2ncc(Cl)c(N[C@H](CO)c3cccc(F)c3)n2)cn1] | C₁₇H₁₈ClFN₆O₂ | 392.8 | 5.03 | 393 | E |
| 87 | 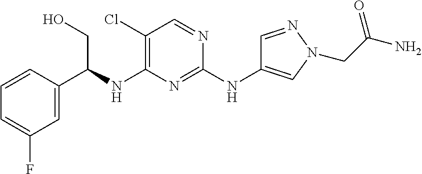 [SMILES: NC(=O)Cn1cc(Nc2ncc(Cl)c(N[C@H](CO)c3cccc(F)c3)n2)cn1] | C₁₇H₁₇ClFN₇O₂ | 405.8 | 4.95 | 406 | E |
| 88 | 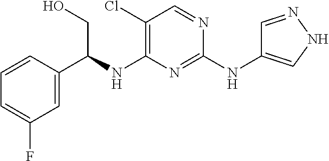 [SMILES: OC[C@@H](Nc1nc(Nc2cn[nH]c2)ncc1Cl)c1cccc(F)c1] | C₁₅H₁₆ClFN₆O | 348.8 | 4.98 | 349 | E |
| 89 | 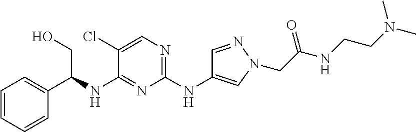 [SMILES: CN(C)CCNC(=O)Cn1cc(Nc2ncc(Cl)c(N[C@H](CO)c3ccccc3)n2)cn1] | C₂₁H₂₇ClN₈O₂ | 459.0 | 0.91 | 459 | B |
| 90 | 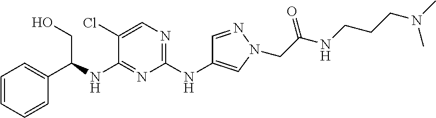 [SMILES: CN(C)CCCNC(=O)Cn1cc(Nc2ncc(Cl)c(N[C@H](CO)c3ccccc3)n2)cn1] | C₂₂H₂₉ClN₈O₂ | 473.0 | 0.93 | 473 | B |
| 91 | 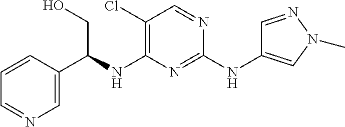 [SMILES: Cn1cc(Nc2ncc(Cl)c(N[C@H](CO)c3cccnc3)n2)cn1] | C₁₅H₁₆ClN₇O | 345.8 | 6.12 | 346 | E |
| 92 | 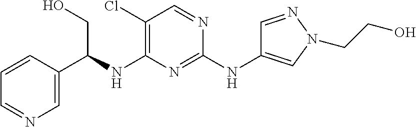 [SMILES: OCCn1cc(Nc2ncc(Cl)c(N[C@H](CO)c3cccnc3)n2)cn1] | C₁₆H₁₈ClN₇O₂ | 375.8 | 5.73 | 376 | B |
| 93 | 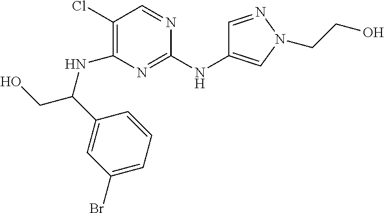 [SMILES: OCCn1cc(Nc2ncc(Cl)c(NC(CO)c3cccc(Br)c3)n2)cn1] | C₁₇H₁₈BrClN₆O₂ | 453.7 | 5.64 | 455 | E |
| 94 | 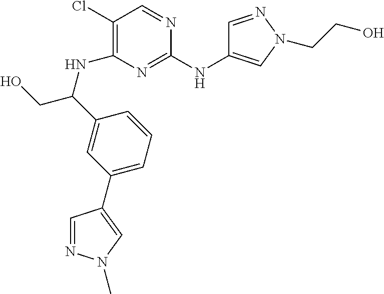 [SMILES: Cn1cc(-c2cccc(C(CO)Nc3nc(Nc4cnn(CCO)c4)ncc3Cl)c2)cn1] | C₂₁H₂₃ClN₈O₂ | 454.9 | 4.87 | 455 | E |
| 95 | 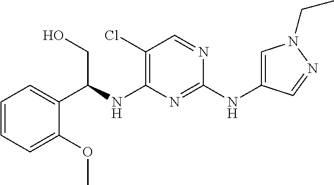 [SMILES: CCn1cc(Nc2ncc(Cl)c(N[C@H](CO)c3ccccc3OC)n2)cn1] | C₁₈H₂₁ClN₆O₂ | 388.9 | 2.27 | 389 | F |
| 96 | 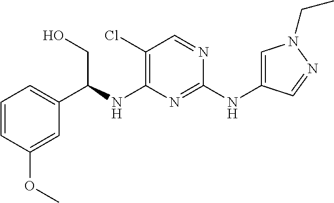 [SMILES: CCn1cc(Nc2ncc(Cl)c(N[C@H](CO)c3cccc(OC)c3)n2)cn1] | C₁₈H₂₁ClN₆O₂ | 388.9 | 2.15 | 389 | F |
| 97 | 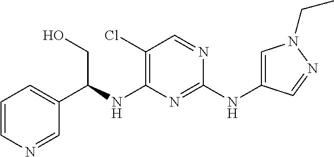 [SMILES: CCn1cc(Nc2ncc(Cl)c(N[C@H](CO)c3cccnc3)n2)cn1] | C₁₆H₁₈ClN₇O | 359.8 | 1.64 | 360 | F |
| 98 | 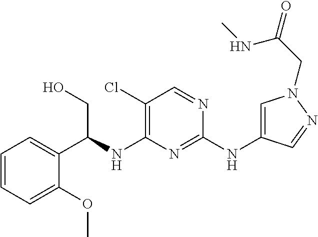 [SMILES: CNC(=O)Cn1cc(Nc2ncc(Cl)c(N[C@H](CO)c3ccccc3OC)n2)cn1] | C₁₉H₂₂ClN₇O₃ | 431.9 | 1.93 | 432 | F |
| 99 | 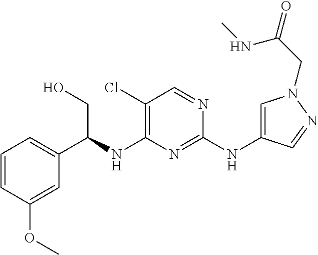 [SMILES: CNC(=O)Cn1cc(Nc2ncc(Cl)c(N[C@H](CO)c3cccc(OC)c3)n2)cn1] | C₁₉H₂₂ClN₇O₃ | 431.9 | 1.81 | 432 | F |
| 100 | 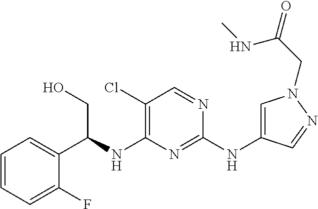 [SMILES: CNC(=O)Cn1cc(Nc2ncc(Cl)c(N[C@H](CO)c3ccccc3F)n2)cn1] | C₁₈H₁₉ClFN₇O₂ | 419.8 | 1.8 | 420 | F |
| 101 | 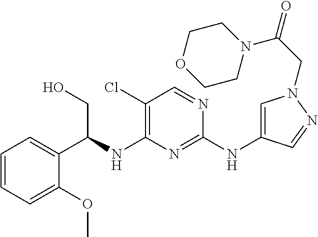 [SMILES: COc1ccccc1[C@@H](CO)Nc1nc(Nc2cnn(CC(=O)N3CCOCC3)c2)ncc1Cl] | C₂₂H₂₆ClN₇O₄ | 487.9 | 2.02 | 488 | F |
| 102 | 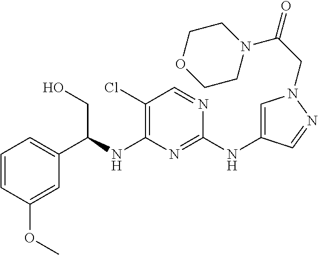 [SMILES: COc1cccc([C@@H](CO)Nc2nc(Nc3cnn(CC(=O)N4CCOCC4)c3)ncc2Cl)c1] | C₂₂H₂₆ClN₇O₄ | 487.9 | 1.89 | 488 | F |
| 103 | 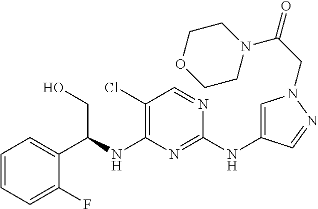 [SMILES: O=C(Cn1cc(Nc2ncc(Cl)c(N[C@H](CO)c3ccccc3F)n2)cn1)N1CCOCC1] | C₂₁H₂₃ClFN₇O₃ | 475.9 | 1.89 | 476 | F |
| 104 | 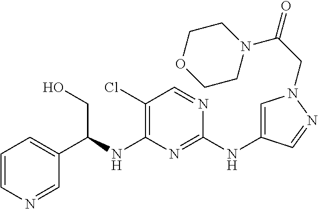 [SMILES: O=C(Cn1cc(Nc2ncc(Cl)c(N[C@H](CO)c3cccnc3)n2)cn1)N1CCOCC1] | C₂₀H₂₃ClN₈O₃ | 458.9 | 1.47 | 459 | F |
| 105 | 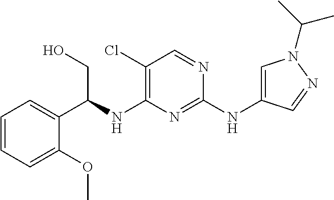 [SMILES: COc1ccccc1[C@@H](CO)Nc1nc(Nc2cnn(C(C)C)c2)ncc1Cl] | C₁₉H₂₃ClN₆O₂ | 402.8 | 2.43 | 403 | F |
| 106 | 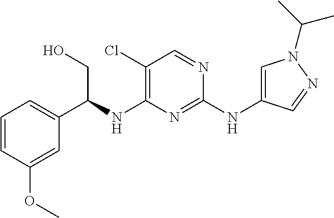 [SMILES: COc1cccc([C@@H](CO)Nc2nc(Nc3cnn(C(C)C)c3)ncc2Cl)c1] | C₁₉H₂₃ClN₆O₂ | 402.8 | 2.29 | 403 | F |
| 107 | 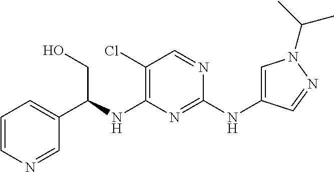 [SMILES: CC(C)n1cc(Nc2ncc(Cl)c(N[C@H](CO)c3cccnc3)n2)cn1] | C₁₇H₂₀ClN₇O | 373.8 | 1.76 | 374 | F |
| 108 | 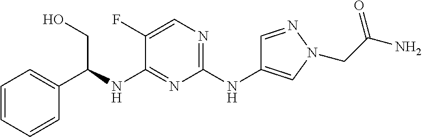 [SMILES: NC(=O)Cn1cc(Nc2ncc(F)c(N[C@H](CO)c3ccccc3)n2)cn1] | C₁₇H₁₈FN₇O₂ | 371.4 | 0.79 | 372 | B |
| 109 | 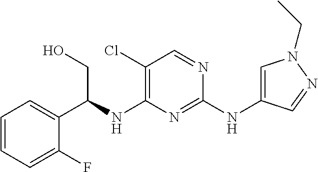 [SMILES: CCn1cc(Nc2ncc(Cl)c(N[C@H](CO)c3ccccc3F)n2)cn1] | C₁₇H₁₈ClFN₆O | 376.8 | 6.01 | 377 | E |
| 110 | 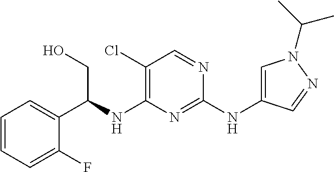 [SMILES: CC(C)n1cc(Nc2ncc(Cl)c(N[C@H](CO)c3ccccc3F)n2)cn1] | C₁₈H₂₀ClFN₆O | 390.8 | 6.74 | 391 | E |
| 111 | 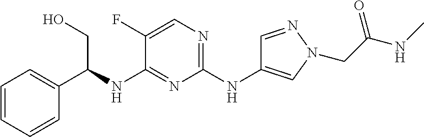 [SMILES: CNC(=O)Cn1cc(Nc2ncc(F)c(N[C@H](CO)c3ccccc3)n2)cn1] | C₁₈H₂₀FN₇O₂ | 385.4 | 0.81 | 386 | B |
| 112 | 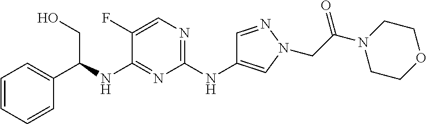 [SMILES: O=C(Cn1cc(Nc2ncc(F)c(N[C@H](CO)c3ccccc3)n2)cn1)N1CCOCC1] | C₂₁H₂₄FN₇O₃ | 441.5 | 0.83 | 442 | B |
| 113 | 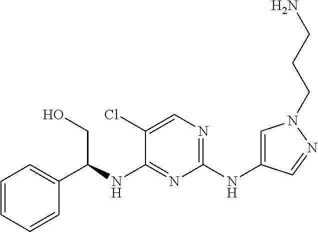 [SMILES: NCCCn1cc(Nc2ncc(Cl)c(N[C@H](CO)c3ccccc3)n2)cn1] | C₁₈H₂₂ClN₇O | 387.9 | 2.1 | 388 | D |
| 114 | 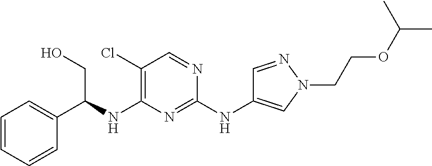 [SMILES: CC(C)OCCn1cc(Nc2ncc(Cl)c(N[C@H](CO)c3ccccc3)n2)cn1] | C₂₀H₂₅ClN₆O₂ | 416.9 | 6.32 | 417 | E |
| 115 | 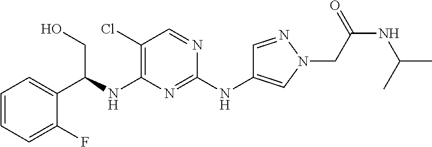 [SMILES: CC(C)NC(=O)Cn1cc(Nc2ncc(Cl)c(N[C@H](CO)c3ccccc3F)n2)cn1] | C₂₀H₂₃ClFN₇O₂ | 447.9 | 5.73 | 448 | E |
| 116 | 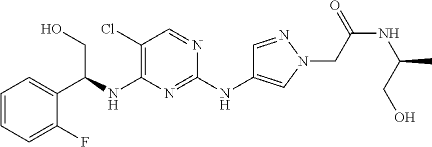 [SMILES: C[C@@H](CO)NC(=O)Cn1cc(Nc2ncc(Cl)c(N[C@H](CO)c3ccccc3F)n2)cn1] | C₂₀H₂₃ClFN₇O₃ | 463.9 | 5.03 | 464 | E |
| 117 | 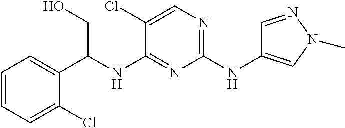 [SMILES: Cn1cc(Nc2ncc(Cl)c(NC(CO)c3ccccc3Cl)n2)cn1] | C₁₆H₁₆Cl₂N₆O | 379.2 | 0.93 | 379 | B |
| 118 | 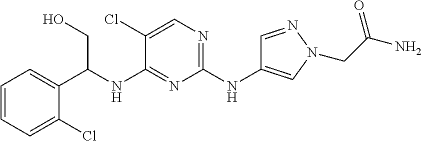 [SMILES: NC(=O)Cn1cc(Nc2ncc(Cl)c(NC(CO)c3ccccc3Cl)n2)cn1] | C₁₇H₁₇Cl₂N₇O₂ | 422.3 | 0.84 | 422 | B |
| 119 | 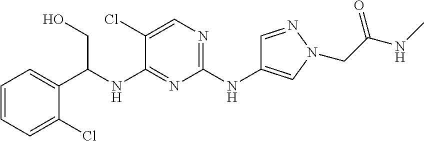 [SMILES: CNC(=O)Cn1cc(Nc2ncc(Cl)c(NC(CO)c3ccccc3Cl)n2)cn1] | C₁₈H₁₉Cl₂N₇O₂ | 436.3 | 0.87 | 436 | B |
| 120 | 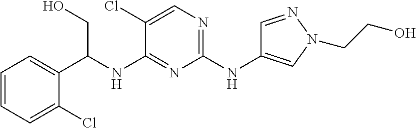 [SMILES: OCCn1cc(Nc2ncc(Cl)c(NC(CO)c3ccccc3Cl)n2)cn1] | C₁₇H₁₈Cl₂N₆O₂ | 409.3 | 0.88 | 409 | B |
| 121 | 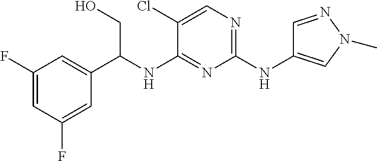 [SMILES: Cn1cc(Nc2ncc(Cl)c(NC(CO)c3cc(F)cc(F)c3)n2)cn1] | C₁₆H₁₅ClF₂N₆O | 380.8 | 0.94 | 381 | B |
| 122 | 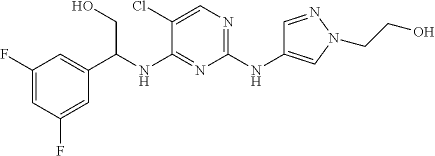 [SMILES: OCCn1cc(Nc2ncc(Cl)c(NC(CO)c3cc(F)cc(F)c3)n2)cn1] | C₁₇H₁₇ClF₂N₆O₂ | 410.8 | 0.89 | 411 | B |
| 123 | 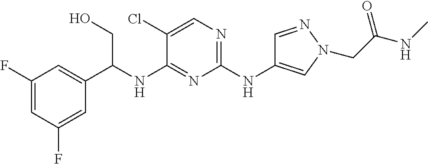 [SMILES: CNC(=O)Cn1cc(Nc2ncc(Cl)c(NC(CO)c3cc(F)cc(F)c3)n2)cn1] | C₁₈H₁₈ClF₂N₇O₂ | 437.8 | 0.88 | 438 | B |
| 124 | 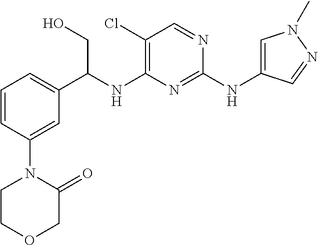 [SMILES: Cn1cc(Nc2ncc(Cl)c(NC(CO)c3cccc(N4CCOCC4=O)c3)n2)cn1] | C₂₀H₂₂ClN₇O₃ | 443.9 | 4.42 | 444 | E |
| 125 | 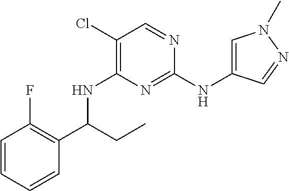 [SMILES: CCC(Nc1nc(Nc2cnn(C)c2)ncc1Cl)c1ccccc1F] | C₁₇H₁₈ClFN₆ | 360.8 | 1.11 | 362 | B |
| 126 | 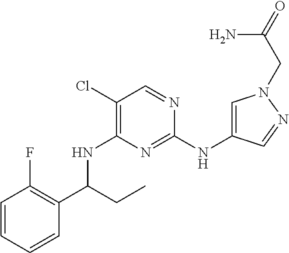 [SMILES: CCC(Nc1nc(Nc2cnn(CC(N)=O)c2)ncc1Cl)c1ccccc1F] | C₁₈H₁₉ClFN₇O | 403.8 | 0.99 | 404 | B |
| 127 | 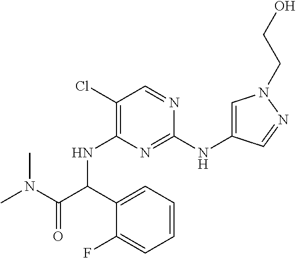 [SMILES: CN(C)C(=O)C(Nc1nc(Nc2cnn(CCO)c2)ncc1Cl)c1ccccc1F] | C₁₉H₂₁ClFN₇O₂ | 433.9 | 5.72 | 434 | E |
| 128 | 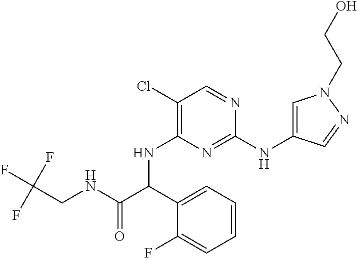 [SMILES: O=C(NCC(F)(F)F)C(Nc1nc(Nc2cnn(CCO)c2)ncc1Cl)c1ccccc1F] | C₁₉H₁₈ClF₄N₇O₂ | 487.8 | 6.93 | 488 | E |
| 129 | 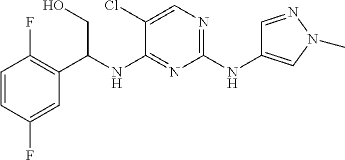 [SMILES: Cn1cc(Nc2ncc(Cl)c(NC(CO)c3cc(F)ccc3F)n2)cn1] | C₁₆H₁₅ClF₂N₆O | 380.8 | 0.93 | 381 | B |
| 130 | 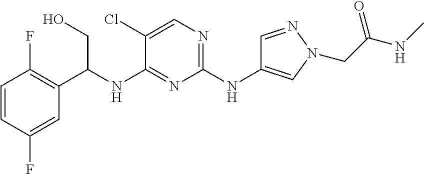 [SMILES: CNC(=O)Cn1cc(Nc2ncc(Cl)c(NC(CO)c3cc(F)ccc3F)n2)cn1] | C₁₈H₁₈ClF₂N₇O₂ | 437.8 | 0.87 | 438 | B |
| 131 | 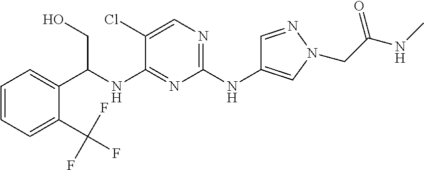 [SMILES: CNC(=O)Cn1cc(Nc2ncc(Cl)c(NC(CO)c3ccccc3C(F)(F)F)n2)cn1] | C₁₉H₁₉ClF₃N₇O₂ | 469.8 | 0.92 | 470 | B |
| 132 | 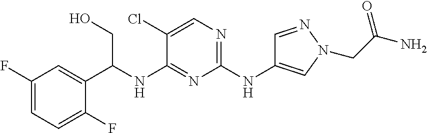 [SMILES: NC(=O)Cn1cc(Nc2ncc(Cl)c(NC(CO)c3cc(F)ccc3F)n2)cn1] | C₁₇H₁₆ClF₂N₇O₂ | 423.8 | 0.85 | 424 | B |
| 133 | 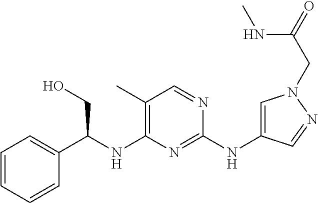 [SMILES: CNC(=O)Cn1cc(Nc2ncc(C)c(N[C@H](CO)c3ccccc3)n2)cn1] | C₁₉H₂₃N₇O₂ | 381.4 | 1.59 | 382 | F |
| 134 | 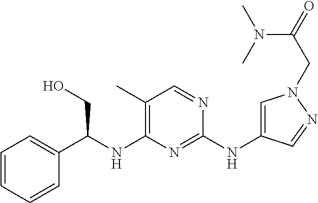 [SMILES: Cc1cnc(Nc2cnn(CC(=O)N(C)C)c2)nc1N[C@H](CO)c1ccccc1] | C₂₀H₂₅N₇O₂ | 395.5 | 1.68 | 396 | F |
| 135 | 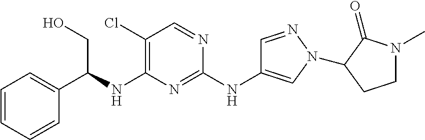 [SMILES: CN1CCC(n2cc(Nc3ncc(Cl)c(N[C@H](CO)c4ccccc4)n3)cn2)C1=O] | C₂₀H₂₂ClN₇O₂ | 427.9 | 5.22 | 428 | E |
| 136 | 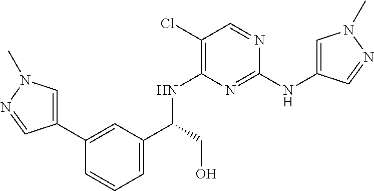 [SMILES: Cn1cc(Nc2ncc(Cl)c(N[C@H](CO)c3cccc(-c4cnn(C)c4)c3)n2)cn1] | C₂₀H₂₁ClN₈O | 424.9 | 5.21 | 425 | E |
| 137 | 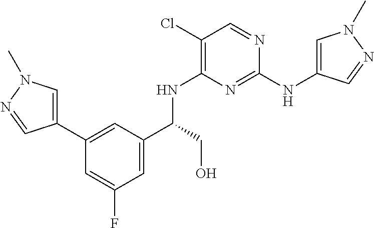 [SMILES: Cn1cc(Nc2ncc(Cl)c(N[C@H](CO)c3cc(F)cc(-c4cnn(C)c4)c3)n2)cn1] | C₂₀H₂₀ClFN₈O | 442.9 | 5.6 | 443 | E |
| 138 | 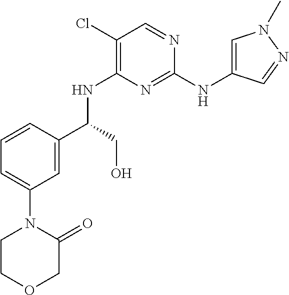 [SMILES: Cn1cc(Nc2ncc(Cl)c(N[C@H](CO)c3cccc(N4CCOCC4=O)c3)n2)cn1] | C₂₀H₂₂ClN₇O₃ | 443.9 | 4.51 | 444 | E |
| 139 | 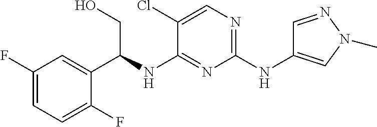 [SMILES: Cn1cc(Nc2ncc(Cl)c(N[C@H](CO)c3cc(F)ccc3F)n2)cn1] | C₁₆H₁₅ClF₂N₆O | 380.8 | 0.93 | 381 | B |
| 140 | 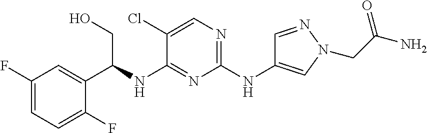 [SMILES: NC(=O)Cn1cc(Nc2ncc(Cl)c(N[C@H](CO)c3cc(F)ccc3F)n2)cn1] | C₁₇H₁₆ClF₂N₇O₂ | 423.8 | 0.85 | 424 | B |
| 141 | 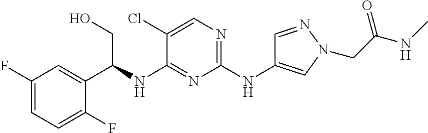 [SMILES: CNC(=O)Cn1cc(Nc2ncc(Cl)c(N[C@H](CO)c3cc(F)ccc3F)n2)cn1] | C₁₈H₁₈ClF₂N₇O₂ | 437.8 | 0.87 | 438 | B |
| 142 | 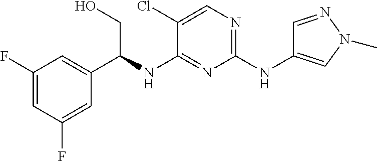 [SMILES: Cn1cc(Nc2ncc(Cl)c(N[C@H](CO)c3cc(F)cc(F)c3)n2)cn1] | C₁₆H₁₅ClF₂N₆O | 380.8 | 0.94 | 381 | B |
| 143 | 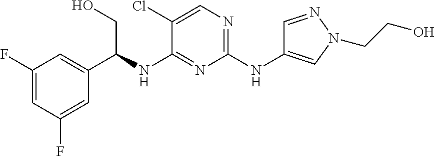 [SMILES: OCCn1cc(Nc2ncc(Cl)c(N[C@H](CO)c3cc(F)cc(F)c3)n2)cn1] | C₁₇H₁₇ClF₂N₆O₂ | 410.8 | 0.89 | 411 | B |
| 144 | 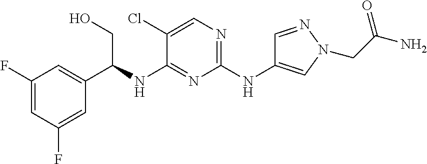 [SMILES: NC(=O)Cn1cc(Nc2ncc(Cl)c(N[C@H](CO)c3cc(F)cc(F)c3)n2)cn1] | C₁₇H₁₆ClF₂N₇O₂ | 423.8 | 0.86 | 424 | B |
| 145 | 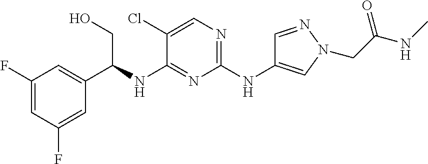 [SMILES: CNC(=O)Cn1cc(Nc2ncc(Cl)c(N[C@H](CO)c3cc(F)cc(F)c3)n2)cn1] | C₁₈H₁₈ClF₂N₇O₂ | 437.8 | 0.88 | 438 | B |
| 146 | 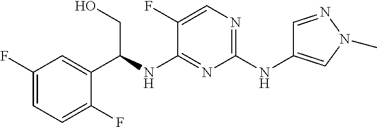 [SMILES: Cn1cc(Nc2ncc(F)c(N[C@H](CO)c3cc(F)ccc3F)n2)cn1] | C₁₆H₁₅F₃N₆O | 364.3 | 0.88 | 365 | B |
| 147 | 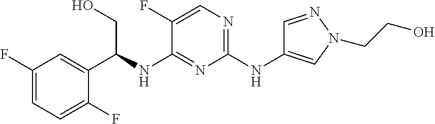 [SMILES: OCCn1cc(Nc2ncc(F)c(N[C@H](CO)c3cc(F)ccc3F)n2)cn1] | C₁₇H₁₇F₃N₆O₂ | 394.4 | 0.83 | 395 | B |
| 148 | 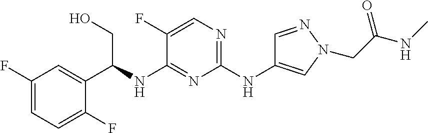 [SMILES: CNC(=O)Cn1cc(Nc2ncc(F)c(N[C@H](CO)c3cc(F)ccc3F)n2)cn1] | C₁₈H₁₈F₃N₇O₂ | 421.4 | 0.82 | 422 | B |
| 149 | 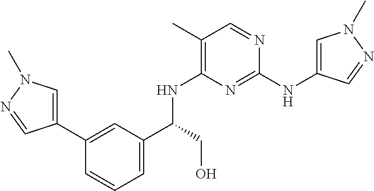 [SMILES: Cc1cnc(Nc2cnn(C)c2)nc1N[C@H](CO)c1cccc(-c2cnn(C)c2)c1] | C₂₁H₂₄N₈O | 404.5 | 5.04 | 405 | E |
| 150 | 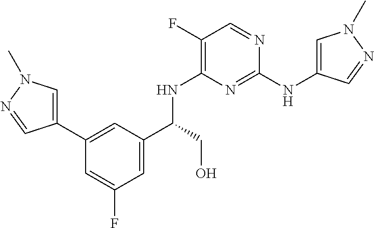 [SMILES: Cn1cc(Nc2ncc(F)c(N[C@H](CO)c3cc(F)cc(-c4cnn(C)c4)c3)n2)cn1] | C₂₀H₂₀F₂N₈O | 426.4 | 5.15 | 427 | E |
| 151 | 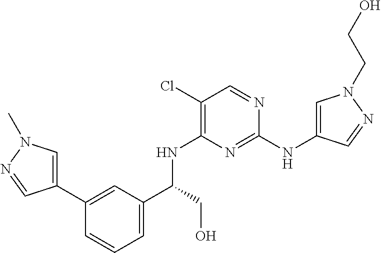 [SMILES: Cn1cc(-c2cccc([C@@H](CO)Nc3nc(Nc4cnn(CCO)c4)ncc3Cl)c2)cn1] | C₂₁H₂₃ClN₈O₂ | 454.9 | 5.07 | 455 | E |
| 152 | 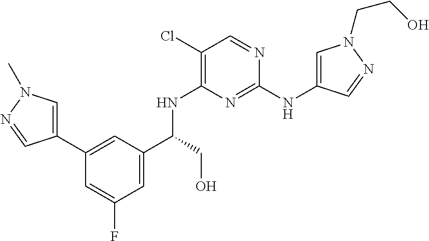 [SMILES: Cn1cc(-c2cc(F)cc([C@@H](CO)Nc3nc(Nc4cnn(CCO)c4)ncc3Cl)c2)cn1] | C₂₁H₂₂ClFN₈O₂ | 472.9 | 5.35 | 473 | E |
| 153 | 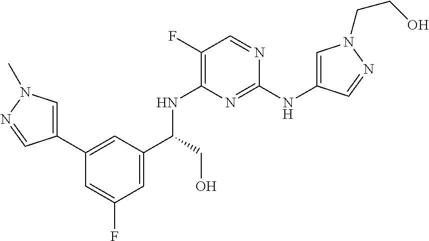 [SMILES: Cn1cc(-c2cc(F)cc([C@@H](CO)Nc3nc(Nc4cnn(CCO)c4)ncc3F)c2)cn1] | C₂₁H₂₂F₂N₈O₂ | 456.4 | 4.93 | 457 | E |
| 154 | 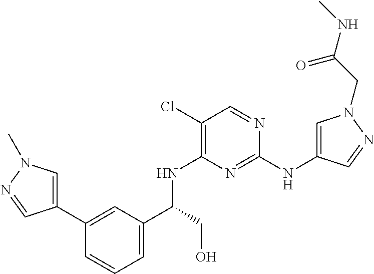 [SMILES: CNC(=O)Cn1cc(Nc2ncc(Cl)c(N[C@H](CO)c3cccc(-c4cnn(C)c4)c3)n2)cn1] | C₂₂H₂₄ClN₉O₂ | 481.9 | 5.7 | 482 | E |
| 155 | 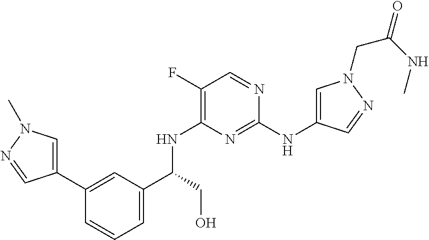 [SMILES: CNC(=O)Cn1cc(Nc2ncc(F)c(N[C@H](CO)c3cccc(-c4cnn(C)c4)c3)n2)cn1] | C₂₂H₂₄FN₉O₂ | 465.5 | 4.66 | 466 | E |
| 156 | 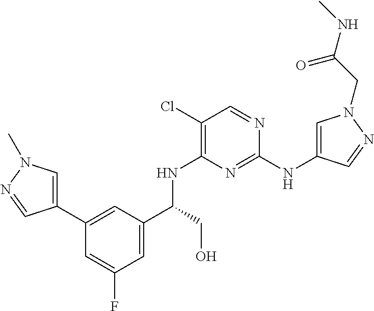 [SMILES: CNC(=O)Cn1cc(Nc2ncc(Cl)c(N[C@H](CO)c3cc(F)cc(-c4cnn(C)c4)c3)n2)cn1] | C₂₂H₂₃ClFN₉O₂ | 499.9 | 5.38 | 500 | E |
| 157 | 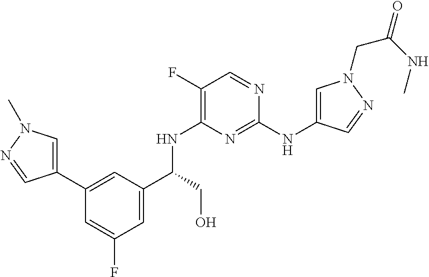 [SMILES: CNC(=O)Cn1cc(Nc2ncc(F)c(N[C@H](CO)c3cc(F)cc(-c4cnn(C)c4)c3)n2)cn1] | C₂₂H₂₃F₂N₉O₂ | 483.5 | 4.88 | 484 | E |
| 158 | 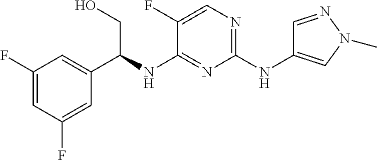 [SMILES: Cn1cc(Nc2ncc(F)c(N[C@H](CO)c3cc(F)cc(F)c3)n2)cn1] | C₁₆H₁₅F₃N₆O | 364.4 | 0.89 | 365 | B |
| 159 | 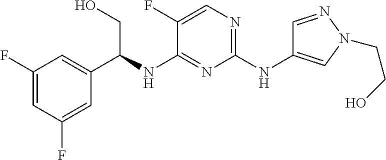 [SMILES: OCCn1cc(Nc2ncc(F)c(N[C@H](CO)c3cc(F)cc(F)c3)n2)cn1] | C₁₇H₁₇F₃N₆O₂ | 394.4 | 0.85 | 395 | B |
| 160 | 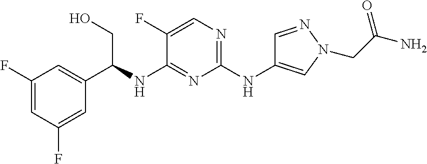 [SMILES: NC(=O)Cn1cc(Nc2ncc(F)c(N[C@H](CO)c3cc(F)cc(F)c3)n2)cn1] | C₁₇H₁₆F₃N₇O₂ | 407.4 | 0.82 | 408 | B |
| 161 | 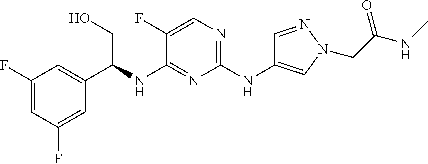 [SMILES: CNC(=O)Cn1cc(Nc2ncc(F)c(N[C@H](CO)c3cc(F)cc(F)c3)n2)cn1] | C₁₈H₁₈F₃N₇O₂ | 421.4 | 0.84 | 422 | B |
| 162 | 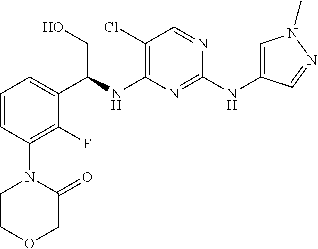 [SMILES: Cn1cc(Nc2ncc(Cl)c(N[C@H](CO)c3cccc(N4CCOCC4=O)c3F)n2)cn1] | C₂₀H₂₁ClFN₇O₃ | 461.9 | 4.76 | 462 | E |
| 163 | 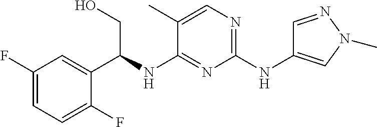 [SMILES: Cc1cnc(Nc2cnn(C)c2)nc1N[C@H](CO)c1cc(F)ccc1F] | C₁₇H₁₈F₂N₆O | 360.4 | 0.89 | 361 | B |
| 164 | 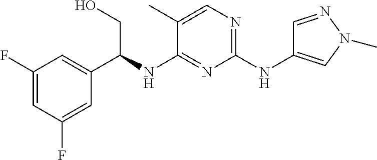 [SMILES: Cc1cnc(Nc2cnn(C)c2)nc1N[C@H](CO)c1cc(F)cc(F)c1] | C₁₇H₁₈F₂N₆O | 360.4 | 0.87 | 361 | B |
| 165 | 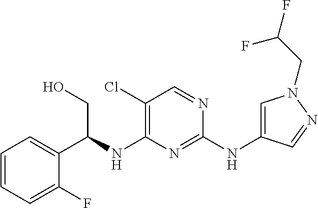 [SMILES: OC[C@@H](Nc1nc(Nc2cnn(CC(F)F)c2)ncc1Cl)c1ccccc1F] | C₁₇H₁₆ClF₃N₆O | 412.8 | 6.26 | 413 | E |
| 166 | 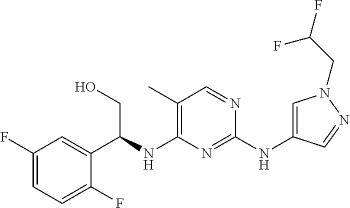 [SMILES: Cc1cnc(Nc2cnn(CC(F)F)c2)nc1N[C@H](CO)c1cc(F)ccc1F] | C₁₈H₁₈F₄N₆O | 410.4 | 5.53 | 411 | E |
| 167 | 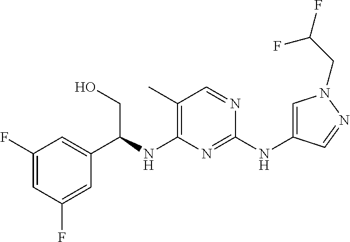 [SMILES: Cc1cnc(Nc2cnn(CC(F)F)c2)nc1N[C@H](CO)c1cc(F)cc(F)c1] | C₁₈H₁₈F₄N₆O | 410.4 | 5.65 | 411 | E |
| 168 | 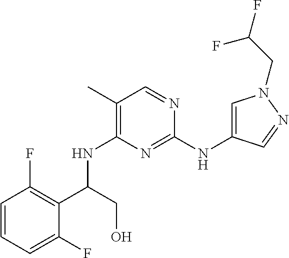 [SMILES: Cc1cnc(Nc2cnn(CC(F)F)c2)nc1NC(CO)c1c(F)cccc1F] | C₁₈H₁₈F₄N₆O | 410.4 | 5.41 | 411 | E |
| 169 | 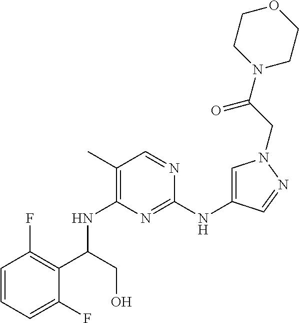 [SMILES: Cc1cnc(Nc2cnn(CC(=O)N3CCOCC3)c2)nc1NC(CO)c1c(F)cccc1F] | C₂₂H₂₅F₂N₇O₃ | 473.5 | 4.97 | 474 | E |
| 170 | 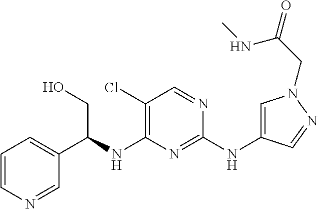 [SMILES: CNC(=O)Cn1cc(Nc2ncc(Cl)c(N[C@H](CO)c3cccnc3)n2)cn1] | C₁₇H₁₉ClN₈O₂ | 402.8 | 1.38 | 403 | F |
| 171 | 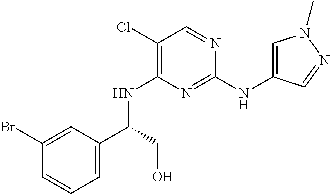 [SMILES: Cn1cc(Nc2ncc(Cl)c(N[C@H](CO)c3cccc(Br)c3)n2)cn1] | C₁₆H₁₆BrClN₆O | 423.7 | 0.97 | 423 | B |
| 172 | 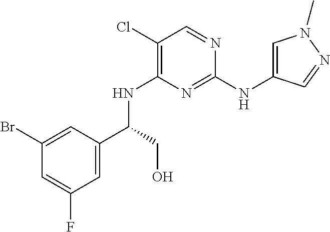 [SMILES: Cn1cc(Nc2ncc(Cl)c(N[C@H](CO)c3cc(F)cc(Br)c3)n2)cn1] | C₁₆H₁₅BrClFN₆O | 441.7 | 1 | 441 | B |
| 173 | 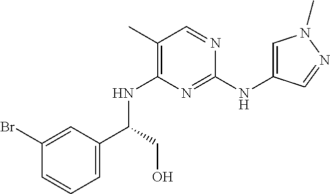 [SMILES: Cc1cnc(Nc2cnn(C)c2)nc1N[C@H](CO)c1cccc(Br)c1] | C₁₇H₁₉BrN₆O | 403.3 | 0.92 | 403 | B |
| 174 | 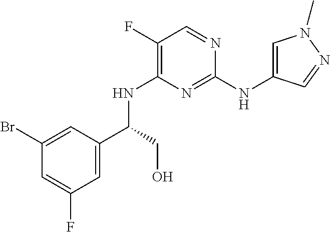 [SMILES: Cn1cc(Nc2ncc(F)c(N[C@H](CO)c3cc(F)cc(Br)c3)n2)cn1] | C₁₆H₁₅BrF₂N₆O | 425.2 | 0.95 | 425 | B |
| 175 | 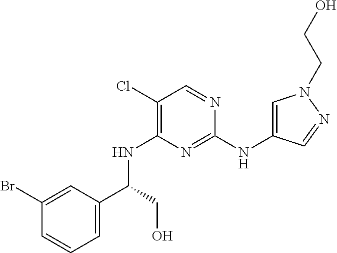 [SMILES: OCCn1cc(Nc2ncc(Cl)c(N[C@H](CO)c3cccc(Br)c3)n2)cn1] | C₁₇H₁₈BrClN₆O₂ | 453.7 | 0.92 | 453 | B |
| 176 | 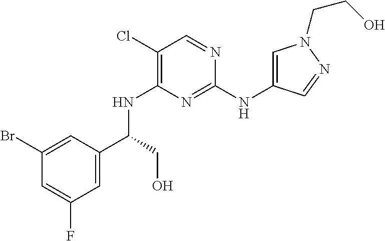 [SMILES: OCCn1cc(Nc2ncc(Cl)c(N[C@H](CO)c3cc(F)cc(Br)c3)n2)cn1] | C₁₇H₁₇BrClFN₆O₂ | 471.7 | 0.94 | 471 | B |
| 177 | 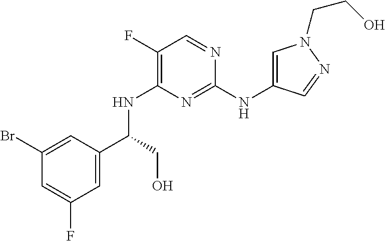 [SMILES: OCCn1cc(Nc2ncc(F)c(N[C@H](CO)c3cc(F)cc(Br)c3)n2)cn1] | C₁₇H₁₇BrF₂N₆O₂ | 455.3 | 0.9 | 455 | B |
| 178 | 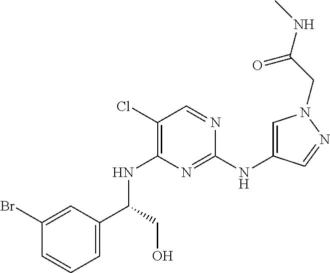 [SMILES: CNC(=O)Cn1cc(Nc2ncc(Cl)c(N[C@H](CO)c3cccc(Br)c3)n2)cn1] | C₁₈H₁₉BrClN₇O₂ | 480.7 | 0.91 | 482 | B |
| 179 | 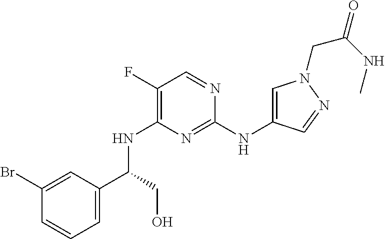 [SMILES: CNC(=O)Cn1cc(Nc2ncc(F)c(N[C@H](CO)c3cccc(Br)c3)n2)cn1] | C₁₈H₁₉BrFN₇O₂ | 464.3 | 0.87 | 464 | B |
| 180 | 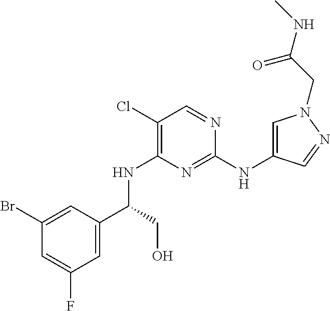 [SMILES: CNC(=O)Cn1cc(Nc2ncc(Cl)c(N[C@H](CO)c3cc(F)cc(Br)c3)n2)cn1] | C₁₈H₁₈BrClFN₇O₂ | 498.7 | 0.87 | 460 | B |
| 181 | 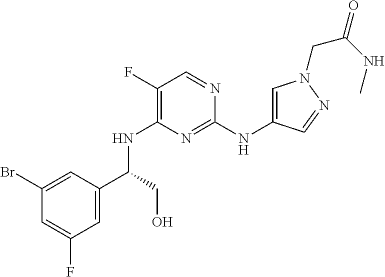 [SMILES: CNC(=O)Cn1cc(Nc2ncc(F)c(N[C@H](CO)c3cc(F)cc(Br)c3)n2)cn1] | C₁₈H₁₈BrF₂N₇O₂ | 482.3 | 0.93 | 500 | B |

### Preparation of (S)-2-(4-((5-chloro-4-((2-hydroxy-1-phenylethyl)amino)pyrimidin-2-yl)amino)-1H-pyrazol-1-yl)-N-methylacetamide (23)

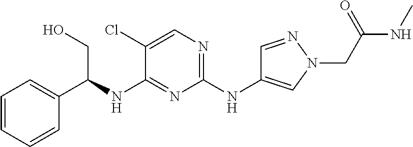 [SMILES: CNC(=O)Cn1cc(Nc2ncc(Cl)c(N[C@H](CO)c3ccccc3)n2)cn1]

### Step 1

To a solution of 4-nitro-1H-pyrazole (100 g, 0.88 mol) in ACN (2 L) was added K₂CO₃ (183.2 g, 1.33 mol) and methyl 2-chloroacetate (95.6 g, 0.88 mol). The mixture was warmed to 60° C. and stirred for 5 h. The mixture was then filtered and solvent removed to give methyl 2-(4-nitro-1H-pyrazol-1-yl)acetate as a white solid (150 g, 92%). 1H NMR (400 MHz CDCl₃): δ 8.28 (s, 1H), 8.11 (s, 1H), 4.98 (s, 2H), 3.84 (s, 3H),

### Step 2

A solution of methyl 2-(4-nitro-1H-pyrazol-1-yl)acetate (150 g, 0.81 mol) and methylamine in ethanol (2 L) was heated to reflux and stirred overnight. The reaction mixture was then allowed to cool and filtered. The filter cake was washed with EtOAc (800 mL) and dried in vacuum to give N-methyl-2-(4-nitro-1H-pyrazol-1-yl)acetamide as a light yellow solid (148 g, 99%). 1H NMR (400 MHz, d6-DMSO): δ 8.82 (s, 1H), 8.24 (s, 1H), 8.15 (d, J=4.4 Hz, 1H), 4.85 (s, 2H), 2.61 (d, J=4.4 Hz, 3H).

### Step 3

To a solution of N-methyl-2-(4-nitro-1H-pyrazol-1-yl)acetamide (80 g, 0.43 mol) in MeOH (1.5 L) was added Pd—C (10%. 16 g) under N₂. The suspension was degassed under vacuum and refilled with H₂ several times. The mixture was stirred under H₂ (50 psi) at 30° C. overnight and then filtered through a pad of Celite, which was washed with MeOH (3×300 mL). The combined filtrate was concentrated to dryness to give 2-(4-amino-1H-pyrazol-1-yl)-N-methylacetamide (441.7 g, yield: 89%, 6 batches) as a red solid, which was used without further purification

### Step 4

To IPA (150 mL) at −78° C. was added 2,4,5-trichloropyrimidine (1.34 g, 7.31 mmol), (S)-2-phenylglycinol (1 g, 7.29 mmol) and DIPEA (2.6 mL, 14.89 mmol). The resulting solution was allowed to warm to room temperature and stirred overnight. The reaction mixture was then poured into stirring water (500 mL) and the resulting white precipitate collected by filtration and dried under vacuum to give (S)-2-((2,5-dichloropyrimidine-4-yl)amino)-2-phenylethanol as a white solid.

1H NMR (400 MHz d6-DMSO): δ 8.21 (s, 1H), 8.04 (d, J=8.0 Hz, 1H), 7.40 (d, J=8 Hz, 2H), 7.33 (t, J=8 Hz, 2H), 7.25 (t, J=8 Hz, 1H), 5.21 (td, J=8.2, 5.2 Hz, 1H), 5.04 (t, J=6 Hz, 1H), 3.87-3.81 (m, 1H), 3.73-3.68 (m, 1H), LC-MS (Method B) RT=0.98 min, (ES⁺) 284.

### Step 4 (Alternative Procedure)

To a stirred solution of 2,4,5-trichloropyrimidine (150 g, 0.818 mol) and (S)-2-phenylglycinol (112.2 g, 0.818 mol) in IPA (1.05 L) at 0° C. was added slowly DIPEA (317.2 g, 2.45 mol). The reaction was stirred at 0° C. for 1 h and allowed to warm up to room temperature. The reaction was stirred overnight at room temperature. TLC R_{f} 0.6 (CH₂Cl₂/CH₃OH=10/1) showed the reaction was complete. The resulting precipitate was filtered, washed with cold IPA, then dried to give (S)-2-((2,5-dichloropyrimidine-4-yl)amino)-2-phenylethanol (550 g, yield: 78.8%, 3 batches) as a white solid.

### Step 5

To (S)-2-((2,5-dichloropyrimidine-4-yl)amino)-2-phenylethanol (0.37 g, 1.30 mmol) and 2-(4-amino-1H-pyrazol-1-yl)-N-methylacetamide (0.2 g, 1.30 mmol) in IPA (15 mL) was added one drop of concentrated hydrochloric acid. The resulting solution was heated in the microwave at 140° C. for 1 h and allowed to stand at room temperature overnight. The resulting white precipitate was collected by filtration and dried under vacuum to give (S)-2-(4-((5-chloro-4-((2-hydroxy-1-phenylethyl)amino)pyrimidin-2-yl)amino)-1H-pyrazol-1-yl)-N-methylacetamide as an off white solid 1H NMR (400 MHz d6-DMSO): δ 10.28 (brs, 1H), 8.62 (brs, 1H), 8.19 (brs, 1H), 8.04 (brs, 1H), 7.74 (brs, 1H), 7.48 (s, 1H), 7.40 (d, J=7.2 Hz, 2H), 7.33 (t, J=7.2 Hz, 2H), 7.25 (t, J=7.2 Hz, 1H), 5.33-5.28 (m, 1H), 4.81-4.71 (m, 3H), 3.89 (dd, J=11.2, 8.6 Hz, 1H), 3.74 (dd, J=11.2, 4.8 Hz, 1H), 2.51 (d, J=2 Hz, 3H), LC-MS (Method B) RT=0.86 min, (ES⁺) 402.

### Step 5 (Alternative Method)

A mixture of (S)-2-((2,5-dichloropyrimidine-4-yl)amino)-2-phenylethanol (100 g, 0.352 mol) and 2-(4-amino-1H-pyrazol-1-yl)-N-methylacetamide (0.352 mol) in IPA (1.5 L) was stirred at 75° C. overnight. TLC R_{f} 0.5 (CH₂Cl₂/CH₃OH=10/1) showed the reaction was complete. The resulting precipitate was filtered, washed with cold IPA, and then dried to give a white solid. Then the white solid was dissolved in water and then adjusted pH=7 with saturated NaHCO₃ aqueous. The resulting precipitate filtered, washed with cold water, and then dried to give the free base. The combined batches of free base (380 g) were dissolved in boiling acetone (20 L). The mixture was filtered to remove insoluble impurities. Then, with rapid stirring, HCI (4 M in dioxane, 250 ml, 1 mol) was added dropwise to the hot solution. The reaction was then allowed to cool to room temperature and filtered. The filtrate cake was washed with cold acetone and dried in vacuo to give (S)-2-(4-((5-chloro-4-((2-hydroxy-1-phenylethyl)amino)pyrimidin-2-yl)amino)-1H-pyrazol-1-yl)-N-methylacetamide as the hydrochloride salt (324 g, yield: 41.3%, 5 batches) as a white solid.

¹H NMR (400 MHz, D₂0): δ 2.600 (s, 3H), 3.817-3.833 (d, J=2.4 Hz, 2H), 4.670-4.673 (m, 2H), 5.097 (5, 1H), 7.175-7.223 (m, 6H), 7.329 (s, IH), 7.723 (s, IH) LCMS: (M+H)⁺ 402

### Preparation of (S)-2-(4-((5-chloro-4-((1-(2-fluorophenyl)-2-hydroxyethyl)amino)pyrimidin-2-yl)amino-1H-pyrazol-1-yl)acetamide (78)

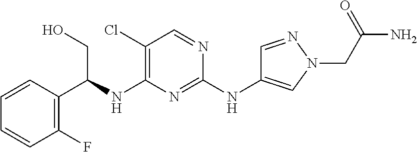 [SMILES: NC(=O)Cn1cc(Nc2ncc(Cl)c(N[C@H](CO)c3ccccc3F)n2)cn1]

### Step 1

To a solution of 4-nitro-1H-pyrazole (2 g, 17.68 mmol) in ACN (200 mL) was added K₂CO₃ (4.9 g, 35.43 mmol) and chloroacetamide (1.66 g, 17.75 mmol). The mixture was warmed to 60° C. and stirred overnight. The mixture was allowed to cool, filtered and the solvent removed to give 2-(4-nitro-1H-pyrazol-1-yl)acetamide as a white solid (3 g, 100%) 1H NMR (400 MHz d6-DMSO): δ 8.82 (s, 1H), 8.26 (s, 1H), 7.67 (brs, 1H), 7.41 (brs, 1H), 4.88 (s, 2H).

### Step 2

To a solution of 2-(4-nitro-1H-pyrazol-1-yl)acetamide (3 g, 17.64 mmol) in MeOH (150 mL) was added Pd—C (10%. 0.3 g) under N₂. The suspension was degassed under vacuum and refilled with H₂ several times. The mixture was stirred under H₂ at atmospheric pressure and room temperature overnight and then filtered through a pad of Celite. The resulting filtrate was concentrated to dryness to give 2-(4-amino-1H-pyrazol-1-yl)acetamide as a burgundy solid, which was used without further purification 1H NMR (400 MHz d6-DMSO): δ 7.17 (brs, 2H), 7.02 (s, 1H), 6.93 (s, 1H), 4.55 (s, 2H), 3.85 (brs, 2H)

### Step 3

To IPA (15 mL) at 0° C. was added DIPEA (1.5 mL, 8.59 mmol) and (S)-2-amino-2-(2-fluorophenyl)ethan-1-ol hydrochloride (0.5 g, 2.61 mmol) followed by 2,4,5-trichloropyrimidine (0.45 g, 2.46 mmol). The resulting solution was allowed to warm to room temperature and stirred overnight. The reaction mixture was then poured into stirring water (50 mL) and the resulting white precipitate collected by filtration and dried under vacuum to give (S)-2-((2,5-dichloropyrimidin-4-yl)amino)-2-(2-fluorophenyl)ethanol as an off white/yellow solid (0.64 g, 86%) which was used without further purification 1H NMR (400 MHz d6-DMSO): δ 8.23 (s, 1H), 8.09 (d, J=8.0 Hz, 1H), 7.48 (td, J=7.6, 1.5 Hz, 1H), 7.41-7.26 (m, 1H), 7.26-7.08 (m, 2H), 5.51 (td, J=8.1, 5.0 Hz, 1H), 5.16 (t, J=5.9 Hz, 1H), 3.92-3.75 (m, 1H), 3.75-3.63 (m, 1H), LC-MS (Method B) RT=1.04 min, (ES⁺) 302.

### Step 4

To IPA (15 mL) was added (S)-2-((2,5-dichloropyrimidin-4-yl)amino)-2-(2-fluorophenyl)ethanol (0.096 g, 0.32 mmol) and 2-(4-amino-1H-pyrazol-1-yl)acetamide (0.07 g, 0.50 mmol). The resulting solution was heated at 80° C. overnight, allowed to cool and solid precipiated by addition of saturated NaHCO3. The resulting collected solid was purified using reverse phase chromatography to give (S)-2-(4-((5-chloro-4-((1-(2-fluorophenyl)-2-hydroxyethyl)amino)pyrimidin-2-yl)amino-1H-pyrazol-1-yl)acetamide as an off white solid 1H NMR (400 MHz d6-DMSO): δ 9.11 (s, 1H), 7.94 (s, 1H), 7.60 (s, 1H), 7.40 (dd, J=15.1, 7.3 Hz, 2H), 7.35-7.25 (m, 2H), 7.23 (s, 1H), 7.21-7.13 (m, 2H), 7.03 (s, 1H), 5.53 (dd, J=13.2, 6.0 Hz, 1H), 5.17 (s, 1H), 4.64 (s, 2H), 3.75 (t, J=5.8 Hz, 2H), LC-MS (Method E) RT=4.86 min, (ES⁺) 406.

### Preparation of (S)-2-(4-((5-chloro-4-((1-(2-fluorophenyl)-2-hydroxyethyl)amino)pyrimidin-2-yl)amino-1H-pyrazol-1-yl)acetamide (78) (Alternative Method)

### Synthetic Route

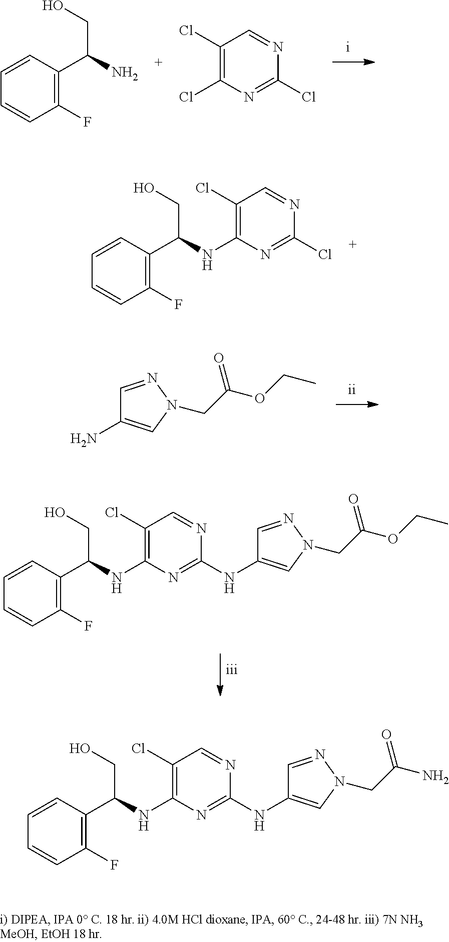 [SMILES: N[C@H](CO)c1ccccc1F ; Clc1ncc(Cl)c(Cl)n1 ; OC[C@@H](Nc1nc(Cl)ncc1Cl)c1ccccc1F ; CCOC(=O)Cn1cc(N)cn1 ; CCOC(=O)Cn1cc(Nc2ncc(Cl)c(N[C@H](CO)c3ccccc3F)n2)cn1 ; NC(=O)Cn1cc(Nc2ncc(Cl)c(N[C@H](CO)c3ccccc3F)n2)cn1]

### (S)-2-((2,5-dichloropyrimidin-4-yl)amino)-2-(2-fluorophenyl)ethanol

A solution of (S)-2-amino-2-(2-fluorophenyl)ethanol hydrochloride (500 mg, 2.62 mmoL) and DIPEA (1.1 mL, 3.0 eq) in IPA (10 mL) were stirred for 10 minutes. The solution was cooled in an ice-water bath then 2,4,5-trichloropyrimidine (450 mg, 2.46 mmol) was added. The reaction mixture was allowed to warm to room temperature and stirred overnight. Water (30 mL) was added and the resultant precipitate was collected by filtration as an off white/yellow solid (0.64 g, 87% yield). UPLC (high pH) RT 1.04 mins m/z 302 (ES+); ¹H NMR (400 MHz, DMSO) δ 8.23 (s, 1H), 8.09 (d, J=8.0 Hz, 1H), 7.48 (td, J=7.6, 1.5 Hz, 1H), 7.41-7.26 (m, 1H), 7.26-7.08 (m, 2H), 5.51 (td, J=8.1, 5.0 Hz, 1H), 5.16 (t, J=5.9 Hz, 1H), 3.92-3.75 (m, 1H), 3.75-3.63 (m, 1H).
(S)-ethyl 2-(4-((5-chloro-4-((1-(2-fluorophenyl)-2-hydroxyethyl)amino)pyrimidin-2-yl)amino)-1H-pyrazol-1-yl)acetate

(S)-2-((2,5-dichloropyrimidin-4-yl)amino)-2-(2-fluorophenyl)ethanol (1.90 g, 6.3 mmol) and ethyl 2-(4-amino-1H-pyrazol-1-yl)acetate (1.60 g, 1.5 eq) were stirred in EtOH (40 mL) with HCl in dioxane (4M, 1.60 mL, 1.0 eq) and heated to 50° C. over 40 hours. The reaction mixture was evaporated to remove half the solvent and then partitioned between EtOAc and NaHCO₃ (aq). The aqueous was extracted with EtOAc, the organics combined washed brine, dried over Na₂SO₄, filtered and evaporated to give 3.0 g of a dark red solid. 169 mg of the red solid was further purified by flash chromatography (silica 10 g, 50-100% EtOAc/Pet Ether) to give 124 mg (73% recovery) colourless solid.

### (S)-2-(4-((5-chloro-4-((1-(2-fluorophenyl)-2-hydroxyethyl)amino)pyrimidin-2-yl)amino)-1H-pyrazol-1-yl)acetamide

(S)-ethyl 2-(4-((5-chloro-4-((1-(2-fluorophenyl)-2-hydroxyethyl)amino)pyrimidin-2-yl)amino)-1H-pyrazol-1-yl)acetate (2.84 g, 6.53 mmol) was dissolved in 7N NH₃ in MeOH (14 mL) and stirred at room temperature for 64 hours. The resulting precipitate was collected by filtration and then triturated with IPA, collected by filtration, washed with IPA and dried in a vacuum oven overnight. LCMS (5) RT 4.81 mins m/z 406 (ES+); ¹H NMR (400 MHz, DMSO) δ 9.11 (s, 1H), 7.94 (s, 1H), 7.60 (s, 1H), 7.40 (dd, J=15.1, 7.3 Hz, 2H), 7.35-7.25 (m, 2H), 7.23 (s, 1H), 7.21-7.13 (m, 2H), 7.03 (s, 1H), 5.53 (dd, J=13.2, 6.0 Hz, 1H), 5.17 (s, 1H), 4.64 (s, 2H), 3.75 (t, J=5.8 Hz, 2H).

% ee determined to be >98%

### (S)-2-(4-((5-chloro-4-((1-(2-fluorophenyl)-2-hydroxyethyl)amino)pyrimidin-2-yl)amino)-1H-pyrazol-1-yl)acetamide (Alternative Reaction Conditions)

(S)-ethyl 2-(4-((5-chloro-4-((1-(2-fluorophenyl)-2-hydroxyethyl)amino)pyrimidin-2-yl)amino)-1H-pyrazol-1-yl)acetate (822 mg, 1.89 mmol) was dissolved in 7N NH₃ in MeOH (15 mL) and heated in the microwave at 120° C. for 30 mins. The reaction mixture was evaporated to give a red solid 718 mg (94% yield). The solid was dissolved in EtOAc with a small amount of MeOH to aid solubility and then washed with NaHCO₃(aq). The aqueous was extracted 2×EtOAC. The organics were combined and washed brine, dried over Na₂SO₄, filtered and evaporated to give 647 mg (84% yield) peach solid.

### HCl Salt Formation

The free-base of compound example 78 (1.02 g, 2.51 mmol) was slurried in IPA (20 mL) at 60° C. To this was added 2M HCl (1.38 mL, 2.76 mmol), resulting in a colourless solution. The solution was allowed to cool to room-temperature before the solvents were gently removed, first by expedited evaporation under a stream of nitrogen, and then in vacuo to give 916 mg (Yield 83%) UPLC high pH RT 1.77 mins m/z 406 (ES+), mpt decomposition 89° C. melt 180° C.

### Biology Assays

### Determination of the Effect of the Compounds According to the Invention on JAK

The compounds of the present invention as described in the previous examples were tested in a Kinobeads™ assay as described for ZAP-70 (WO-A 2007/137867). Briefly, test compounds (at various concentrations) and the affinity matrix with the immobilized aminopyrido-pyrimidine ligand 24 were added to cell lysate aliquots and allowed to bind to the proteins in the lysate sample. After the incubation time the beads with captured proteins were separated from the lysate. Bound proteins were then eluted and the presence of JAK1, JAK2, JAK3 and TYK2 was detected and quantified using specific antibodies in a dot blot procedure and the Odyssey infrared detection system. Dose response curves for individual kinases were generated and IC₅₀ values calculated. Kinobeads™ assays for ZAP-70 (WO-A 2007/137867) and for kinase selectivity profiling (WO-A 2006/134056) have been previously described.

### Protocols

### Washing of Affinity Matrix

The affinity matrix was washed two times with 15 mL of lx DP buffer containing 0.2% NP40 (IGEPAL® CA-630, Sigma, #13021) and then resuspended in 1×DP buffer containing 0.2% NP40 (3% beads slurry).

5×DP buffer: 250 mM Tris-HCl pH 7.4, 25% Glycerol, 7.5 mM MgCl₂, 750 mM NaCl, 5 mM Na₃VO₄; filter the 5×DP buffer through a 0.22 μm filter and store in aliquots at −80° C. The 5×DP buffer is diluted with H₂O to 1×DP buffer containing 1 mM DTT and 25 mM NaF.

### Preparation of Test Compounds

Stock solutions of test compounds were prepared in DMSO. In a 96 well plate 304 solution of diluted test compounds at 5 mM in DMSO were prepared. Starting with this solution a 1:3 dilution series (9 steps) was prepared. For control experiments (no test compound) a buffer containing 2% DMSO was used.

### Cell Culture and Preparation of Cell Lysates

Molt4 cells (ATCC catalogue number CRL-1582) and Ramos cells (ATCC catalogue number CRL-1596) were grown in 1 L Spinner flasks (Integra Biosciences, #182101) in suspension in RPMI 1640 medium (Invitrogen, #21875-034) supplemented with 10% Fetal Bovine Serum (Invitrogen) at a density between 0.15×10⁶ and 1.2×10⁶ cells/mL. Cells were harvested by centrifugation, washed once with 1×PBS buffer (Invitrogen, #14190-094) and cell pellets were frozen in liquid nitrogen and subsequently stored at −80° C. Cells were homogenized in a Potter S homogenizer in lysis buffer: 50 mM Tris-HCl, 0.8% NP40, 5% glycerol, 150 mM NaCl, 1.5 mM MgCl₂, 25 mM NaF, 1 mM sodium vanadate, 1 mM DTT, pH 7.5. One complete EDTA-free tablet (protease inhibitor cocktail, Roche Diagnostics, 1873580) per 25 mL buffer was added. The material was dounced 10 times using a mechanized POTTER S, transferred to 50 mL falcon tubes, incubated for 30 minutes on ice and spun down for 10 minutes at 20,000 g at 4° C. (10,000 rpm in Sorvall SLA600, precooled). The supernatant was transferred to an ultracentrifuge (UZ)-polycarbonate tube (Beckmann, 355654) and spun for 1 hour at 100.000 g at 4° C. (33.500 rpm in Ti50.2, precooled). The supernatant was transferred again to a fresh 50 mL falcon tube, the protein concentration was determined by a Bradford assay (BioRad) and samples containing 50 mg of protein per aliquot were prepared. The samples were immediately used for experiments or frozen in liquid nitrogen and stored frozen at −80° C.

### Dilution of Cell Lysate

Cell lysate (approximately 50 mg protein per plate) was thawed in a water bath at room temperature and then stored on ice. To the thawed cell lysate 1×DP 0.8% NP40 buffer containing protease inhibitors (1 tablet for 25 mL buffer; EDTA-free protease inhibitor cocktail; Roche Diagnostics 1873580) was added in order to reach a final protein concentration of 10 mg/mL total protein. The diluted cell lysate was stored on ice. Mixed Molt4/Ramos lysate was prepared by combining one volume of Molt4 lysate and two volumes of Ramos lysate (ratio 1:2).

Incubation of Lysate with Test Compound and Affinity Matrix

To a 96 well filter plate (Multiscreen HTS, BV Filter Plates, Millipore #MSBVN1250) were added per well: 100 μL affinity matrix (3% beads slurry), 34, of compound solution, and 50 μL of diluted lysate. Plates were sealed and incubated for 3 hours in a cold room on a plate shaker (Heidolph tiramax 1000) at 750 rpm. Afterwards the plate was washed 3 times with 230 μL washing buffer (1×DP 0.4% NP40). The filter plate was placed on top of a collection plate (Greiner bio-one, PP-microplate 96 well V-shape, 65120) and the beads were then eluted with 20 μL of sample buffer (100 mM Tris, pH 7.4, 4% SDS, 0.00025% bromophenol blue, 20% glycerol, 50 mM DTT). The eluate was frozen quickly at −80° C. and stored at −20° C.

### Detection and Quantification of Eluted Kinases

The kinases in the eluates were detected and quantified by spotting on nitrocellulose membranes and using a first antibody directed against the kinase of interest and a fluorescently labelled secondary antibody (anti-rabbit IRDye™ antibody 800 (Licor, #926-32211). The Odyssey Infrared Imaging system from LI-COR Biosciences (Lincoln, Nebr., USA) was operated according to instructions provided by the manufacturer (Schutz-Geschwendener et al., 2004. Quantitative, two-color Western blot detection with infrared fluorescence. Published May 2004 by LI-COR Biosciences, www.licor.com).

After spotting of the eluates the nitrocellulose membrane (BioTrace NT; PALL, #BTNT30R) was first blocked by incubation with Odyssey blocking buffer (LICOR, 927-40000) for 1 hour at room temperature. Blocked membranes were then incubated for 16 hours at the temperature shown in table 4 with the first antibody diluted in Odyssey blocking buffer (LICOR #927-40000). Afterwards the membrane was washed twice for 10 minutes with PBS buffer containing 0.2% Tween 20 at room temperature. The membrane was then incubated for 60 minutes at room temperature with the detection antibody (anti-rabbit IRDye™ antibody 800, Licor, #926-32211) diluted in Odyssey blocking buffer (LICOR #927-40000). Afterwards the membrane was washed twice for 10 minutes each with 1×PBS buffer containing 0.2% Tween 20 at room temperature. Then the membrane was rinsed once with PBS buffer to remove residual Tween 20. The membrane was kept in PBS buffer at 4° C. and then scanned with the Odyssey instrument. Fluorescence signals were recorded and analysed according to the instructions of the manufacturer.

TABLE 7
Sources and dilutions of antibodies

| | Primary antibody | Temp of Primary | |
|---|---|---|---|
| Target kinase | (dilution) | incubation | Secondary antibody (dilution) |
| JAK1 | Cell signalling #3332 | 4° C. | Licor anti-rabbit 800 (1:15000) |
| | 1:100 | | |
| JAK2 | Cell signalling #3230 | Room temperature | Licor anti-rabbit 800 (1:15000) |
| | (1:100) | | |
| JAK3 | Cell signalling #3775 | 4° C. | Licor anti-rabbit 800 (1:5000) |
| | (1:100) | | |
| TYK2 | Cell signalling #06-638 | Room temperature | Licor anti-rabbit 800 (1:5000) |
| | (1:1000) | | |

TABLE 8
Inhibition values (IC₅₀ in μM) as determined in the
Kinobeads ™ assay (Activity level: A < 0.1 μM;
0.1 μM ≦ B < 1 μM; 1 μM ≦ C < 10 μM; D ≧ 10 μM).

| Example | JAK1 | JAK2 | JAK3 | TYK2 |
|---|---|---|---|---|
| 1 | C | D | A | C |
| 2 | D | D | B | C |
| 3 | C | D | B | C |
| 4 | C | C | A | C |
| 5 | C | C | B | D |
| 6 | D | D | A | D |
| 7 | C | D | B | C |
| 8 | D | D | B | D |
| 9 | B | C | A | C |
| 10 | C | D | A | C |
| 11 | D | D | B | D |
| 12 | C | D | B | C |
| 13 | C | B | A | B |
| 14 | C | C | B | C |
| 15 | C | D | B | C |
| 16 | C | D | B | C |
| 17 | D | D | B | D |
| 18 | C | C | A | C |
| 19 | B | C | A | C |
| 20 | C | D | B | C |
| 21 | C | C | A | B |
| 22 | B | C | A | B |
| 23 | B | C | A | C |
| 24 | C | D | A | C |
| 25 | C | D | B | D |
| 26 | C | C | A | C |
| 27 | C | D | A | C |
| 28 | C | D | A | C |
| 29 | C | C | A | C |
| 30 | B | C | A | B |
| 31 | C | C | A | C |
| 32 | C | C | A | C |
| 33 | C | C | A | C |
| 34 | C | C | B | D |
| 35 | C | C | B | C |
| 36 | B | C | A | B |
| 37 | C | C | A | C |
| 38 | B | C | A | C |
| 39 | B | B | A | B |
| 40 | C | C | A | C |
| 41 | D | D | B | D |
| 42 | C | C | A | B |
| 43 | B | C | A | B |
| 44 | C | C | A | C |
| 45 | D | D | B | C |
| 46 | C | C | A | C |
| 47 | B | C | A | C |
| 48 | B | C | A | C |
| 49 | C | D | B | D |
| 50 | D | D | B | D |
| 51 | D | D | B | D |
| 52 | C | D | B | C |
| 53 | D | D | B | D |
| 54 | D | D | C | C |
| 55 | D | D | B | D |
| 56 | B | C | A | B |
| 57 | C | D | A | C |
| 58 | C | C | A | C |
| 59 | D | D | B | D |
| 60 | C | D | B | C |
| 61 | C | C | A | C |
| 62 | C | D | A | C |
| 63 | C | D | C | C |
| 64 | C | C | A | C |
| 65 | C | C | B | C |
| 66 | C | C | B | C |
| 67 | B | C | A | B |
| 68 | B | C | A | B |
| 69 | D | D | B | D |
| 70 | B | C | A | B |
| 71 | C | D | B | C |
| 72 | C | D | B | C |
| 73 | C | C | A | C |
| 74 | B | C | A | C |
| 75 | C | C | A | C |
| 76 | B | C | A | B |
| 77 | B | C | A | B |
| 78 | A | C | A | B |
| 79 | B | C | A | C |
| 80 | B | C | A | B |
| 81 | B | C | A | B |
| 82 | C | D | A | C |
| 83 | C | C | A | C |
| 84 | C | D | A | C |
| 85 | B | B | A | B |
| 86 | B | B | A | B |
| 87 | A | B | A | B |
| 88 | B | B | A | B |
| 89 | B | C | A | B |
| 90 | B | C | A | B |
| 91 | B | C | A | B |
| 92 | B | C | A | B |
| 93 | C | C | B | B |
| 94 | C | D | B | D |
| 95 | B | B | A | C |
| 96 | C | D | A | C |
| 97 | C | C | A | C |
| 98 | B | C | A | B |
| 99 | C | D | A | C |
| 100 | B | C | A | C |
| 101 | B | C | A | A |
| 102 | B | D | A | B |
| 103 | B | C | A | B |
| 104 | B | C | A | B |
| 105 | C | B | A | C |
| 106 | D | C | B | C |
| 107 | C | C | A | C |
| 108 | C | D | B | C |
| 109 | C | C | A | C |
| 110 | C | B | A | C |
| 111 | C | D | A | C |
| 112 | C | D | A | B |
| 113 | C | C | A | C |
| 114 | C | C | A | C |
| 115 | B | C | A | B |
| 116 | B | C | A | B |
| 117 | C | C | A | C |
| 118 | B | C | A | B |
| 119 | B | C | A | C |
| 120 | B | C | A | B |
| 121 | B | C | A | C |
| 122 | B | B | A | B |
| 123 | B | C | A | C |
| 124 | C | D | A | C |
| 125 | C | C | B | C |
| 126 | B | C | B | C |
| 127 | C | C | B | C |
| 128 | C | D | B | C |
| 129 | B | B | A | B |
| 130 | A | B | A | B |
| 131 | C | C | B | B |
| 132 | A | B | A | B |
| 133 | C | C | A | C |
| 134 | B | C | A | C |
| 135 | B | C | A | C |
| 136 | D | D | B | D |
| 137 | C | C | B | C |
| 138 | C | D | A | C |
| 139 | B | B | A | B |
| 140 | A | B | A | B |
| 141 | A | B | A | B |
| 142 | B | B | A | C |
| 143 | A | B | A | B |
| 144 | A | B | A | B |
| 145 | A | B | A | B |
| 146 | B | C | A | B |
| 147 | B | B | A | B |
| 148 | B | C | A | B |
| 149 | D | D | B | D |
| 150 | C | D | B | D |
| 151 | C | D | B | D |
| 152 | B | C | A | C |
| 153 | C | D | B | D |
| 154 | C | C | A | B |
| 155 | D | D | B | D |
| 156 | C | C | A | D |
| 157 | C | D | B | D |
| 158 | C | C | A | C |
| 159 | B | C | A | B |
| 160 | B | C | A | B |
| 161 | B | C | A | B |
| 162 | C | D | A | D |
| 163 | B | B | A | B |
| 164 | B | C | B | C |
| 165 | B | C | A | C |
| 166 | B | C | B | C |
| 167 | C | C | B | C |
| 168 | C | D | B | C |
| 169 | C | C | A | B |
| 170 | C | C | A | C |

### Cell Assays

pSTAT5 Assay

### Assay Principle

STAT5 phosphorylation represents one of the proximal events in the signalling cascade downstream of JAK3 activation. Therefore STAT5 phosphorylation is an appropriate readout to assess the mechanistic effect of JAK3 inhibition. Stimulation of human YT cells, an NK-like cell line, with interleukin-2 (IL-2) results in phosphorylation of STAT5 at tyrosine residue 694 (Tyr694) that can be quantitatively measured by immunodetection with specific antibodies and an appropriate detection method, in this case AlphaScreen assay technology.

### Assay Protocol

### Cell Culture and Cell Seeding

Human YT cells were grown in RPMI medium (Lonza, BE12-167) with 2 mM L-Glutamine (Invitrogen, 25030-024) and 10% heat-inactivated FBS (Invitrogen, 10106-169) and kept in a humidified incubator (37° C., 5% CO₂). Cells were harvested by centrifugation, washed once with HBSS (Invitrogen, 14180-046), resuspended in HBSS at 1.5×10⁶ cells/ml and 0.9×10⁴ cells were seeded in 6 μl per well in a 96 well White plate (PerkinElmer, 6005569).

Treatment with Test Compounds and IL-2 Stimulation

Test compounds were dissolved in DMSO and a 1:3 dilution series (9 steps) was prepared. To generate a dose response curve, 3 μl of fourfold concentrated compound in 4% DMSO/HBSS were added to each cell sample in the 96 well plate resulting in a final DMSO concentration of 1% DMSO. Cells were incubated for one hour in a humidified incubator (37° C., 5% CO₂). To each well 3 μl of a fourfold concentrated IL-2 solution (Recombinant human IL-2, Peprotech 200-02; 120 nM solution in HBSS) was added and incubated for 30 minutes at room temperature. Cells were lysed by adding 3 μl of 5× lysis buffer (SureFire lysis buffer; Perkin Elmer, TGRS5S10K) and incubated for 10 minutes at room temperature with gentle shaking

### Signal Detection

For signal detection by AlphaScreen® technology the SureFire phospho-STAT5 (Tyr694/Tyr699) kit was used according to instructions provided by the manufacturer (Perkin Elmer, TGRS5S10K). Acceptor beads were added as recommended by the manufacturer (Reactivation buffer/Activation buffer/Acceptor beads at a ratio of 40:10:1) and incubated at room temperature for 1.5 hours with gentle shaking Then donor beads were added as recommended (Dilution buffer/Donor beads at a ratio of 20:1) and incubated at room temperature for 1.5 hours with gentle shaking Plates were read on an Envision instrument (Perkin Elmer) with the AlphaScreen protocol. Data were analysed in BioAssay using the nonlinear regression for a sigmoidal dose-response with a variable slope.

Table 9 provides data for selected compounds of the invention in the pSTAT5 cell assay.

TABLE 9
Inhibition values (IC₅₀ in μM) as determined in the
pSTAT5 cell assay (Activity level: A < 0.25 μM;
0.25 μM ≦ B < 1 μM; 1 μM ≦ C < 10 μM; D ≧ 10 μM).

| Example | pSTAT5 |
|---|---|
| 4 | B |
| 9 | B |
| 23 | B |
| 30 | B |
| 67 | B |
| 68 | B |
| 75 | B |
| 78 | A |
| 87 | A |
| 165 | A |

1. A compound of formula (I) 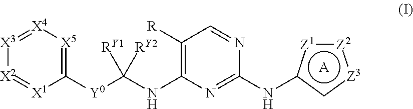
or a pharmaceutically acceptable salt thereof, wherein
R is H; F; Cl; Sr; CN; CH₃; CF₃ or C(O)NH₂;
Ring A is a 5 membered aromatic heterocycle in which Z¹, Z², and Z³ are independently selected from the group consisting of C(R¹); N; and N(R¹), provided that at least one of Z¹, Z², Z³ is N or N(R¹);
Each R¹ is independently H, halogen; CN; C(O)OR²; OR²; C(O)R²; C(O)N(R²R^{2a}); S(O)₂N(R²R^{2a}); S(O)N(R²R^{2a}); S(O)₂R²; S(O)R²; N(R²)S(O)₂N(R^{2a}R^{2b}); N(R²)S(O)N(R^{2a}R^{2b}); SR²; N(R²R^{2a}); NO₂; OC(O)R²; N(R²)C(O)R^{2a}; N(R²)S(C)₂R^{2a}; N(R²)S(O)R^{2a}; N(R²)C(O)N(R^{2a}R^{b2}); N(R²)C(O)OR^{2a}; OC(O)N(R²R^{2a}); T¹; C₁₋₆ alkyl; C₂₋₆ alkenyl; or C₂₋₆ alkynyl, wherein C₁₋₆ alkyl: C₂₋₆ alkenyl; and C₂₋₆alkynyl are optionally substituted with one or more R³, which are the same or different;
R², R^{2a}, R^{2b} are independently selected from the group consisting of H; T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R³, which are the same or different;
R³ is halogen; CN; C(O)OR⁴; OR⁴; C(O)R⁴; C(O)N(R⁴R^{4a}); S(O)₂N(R⁴R^{4a}); S(O)N(R⁴R^{4a}); S(O)₂R⁴; S(O)R⁴; N(R⁴)S(O)₂N(R^{4a}R^{4b}); N(R⁴)S(O)N(R^{4a}R^{4b}); SR⁴; N(R⁴R^{4a}); NO₂; OC(O)R⁴; N(R⁴)C(O)R^{4a}; N(R⁴)S(O)₂R^{4a}; N(R⁴)S(O)R^{4a}; N(R⁴)C(O)N(R^{4a}R^{4b}); N(R⁴)C(O)OR^{4a}; OC(O)N(R⁴R^{4a}); or T¹;
R⁴, R^{4a}, R^{4b} are independently selected from the group consisting of H; T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁵, which are the same or different;
R⁵ is halogen; CN; C(O)OR^{5a}; OR^{5a}; C(O)R^{5a}; C(O)R^{5a}; C(O)N(R^{5a}R^{5b}); S(O)₂N(R^{5a}R^{5b}); S(O)N(R^{5a}R^{5b}); S(O)₂R^{5a}; S(O)R^{5a}; N(R^{5a})S(O)₂N(R^{5b}R^{5c}); N(R^{5a})S(O)N(R^{5b}R^{5c}); SR^{5a}; N(R^{5a}R^{5b}); NO₂; OC(O)R^{5a}; N(R^{5a})C(O)R^{5a}; N(R^{5a})S(O)₂R^{5b}; N(R^{5a})S(O)R^{5b}; N(R^{5a})C(O)N(R^{5b}R^{5c}); N(R^{5a})C(O)OR^{5b}; or OC(O)N(R^{5a}R^{5b});
R^{5a}, R^{5b}, R^{5c} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen; which are the same or different;
T¹ is C₃₋₇ cycloalkyl; saturated 4 to 7 membered heterocyclyl; or 7 to 11 membered heterobicyclyl, wherein T¹ is optionally substituted with one or more R¹⁰, which are the same or different;
Y⁰ is (CR^{Y3}R^{Y4})ₙ;
n is 0; or 1;
One of R^{Y1}; R^{Y2}; R^{Y3}; R^{Y4} is R^{Y0} and the others are selected from the group consisting of H; CH₃; and CF₃;
R^{Y0} is unsubstituted C₁₋₄ alkyl; CH₂CH₂OR^{Y5}; CH₂CH₂C(O)T^{Y1}; CH₂CH₂C(O)OR^{Y5}; CH₂CH₂OC(O)R^{Y5}; CH₂CH₂N(R^{Y5}R^{5a}); CH₂CH₂N(R^{Y5})C(O)R^{Y5a}; CH₂CH₂C(O)N(R^{Y5}R^{Y5a}); CH₂OR^{Y5}; CH₂C(O)T^{Y1}; CH₂C(O)OR^{Y5}; CH₂OC(O)R^{Y5}; CH₂N(R^{Y5}R^{Y58}); CH₂N(R^{Y5})C(O)R^{Y5a}; CH₂C(O)N(R^{Y5}R^{Y5a}); C(O)T^{Y1}; C(O)OR^{Y5}; or C(O)N(R^{Y5}R^{Y5a});
R^{Y5}, R^{Y5a} are independently selected from the group consisting of H; T^{Y1}; and C₁₋₄ alkyl, wherein C₁₋₄ alkyl is optionally substituted with one or more R^{Y6}, which are the same or different;
R^{Y6} is halogen; OR^{Y7}; C(O)T^{Y1}; C(O)OR^{Y7}; OC(O)R^{Y7}; N(R^{Y7}R^{Y7a}); or N(R^{Y7})C(O)R^{Y7a};
R^{Y7}; R^{Y7a} are independently selected from the group consisting of H, C₁₋₄ alkyl; or T^{Y1}, wherein C₁₋₄ alkyl is optionally substituted with one or more halogen, which are the same or different;
T^{Y1} is unsubstituted C₃₋₇ cycloalkyl; unsubstituted saturated 4 to 7 membered heterocyclyl; or saturated 7 to 11 membered heterobicyclyl;
X¹ is C(R^{6a}) or N; X² is C(R^{6b}) or N; X³ is CH, CF, COH or N; X⁴ is CR^{6c}) or N; X⁵ is C(R^{6d}) or N, provided that at most two of X¹, X². X⁴, X⁵ are N;
R^{6a}, R^{6b}, R^{6c}, R^{6d} are independently selected from the group consisting of H; halogen; CN; C(O)OR⁷; OR⁷; C(O)R⁷; C(O)N(R⁷R^{7a}); S(O)₂N(R⁷R^{7a}); S(O)N(R⁷R^{7a}); S(O)₂R⁷; S(O)R⁷; N(R⁷)S(O)₂N(R^{7a}R^{7b}); N(R⁷)S(O)N(R^{7a}R^{7b}); SR⁷; N(R⁷R^{7a}); NO₂; OC(O)R⁷; N(R⁷)C(O)R^{7a}; N(R⁷)S(O)₂R^{7a}; N(R⁷)S(O)R^{7a}, N(R⁷)C(O)N(R^{7a}R^{7b}); N(R⁷)C(O)OR^{7a}; OC(O)N(R⁷R^{7a}); T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹¹, which are the same or different;
Optionally the pair R^{6a}/R^{6b} is joined to form a ring T³;
R⁷, R^{7a}, R^{7b} are independently selected from the group consisting of H; CN; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁸, which are the same or different;
R⁸ is halogen; CM; C(O)OR⁹; OR⁹; C(O)R⁹; C(O)N(R⁹R^{9a}); S(O)₂N(R⁹R^{9a}); S(O)N(R⁹R^{9a}); S(O)₂R⁹; S(O)R⁹; N(R⁹)S(O)₂N(R^{9a}R^{9b}); N(R⁹)S(O)N(R^{9a}R^{9b}); SR⁹; N(R⁹R^{9a}); NO₂; OC(O)R⁹; N(R⁹)C(O)R^{9a}; N(R⁹)S(O)₂R^{9a}; N(R⁹)S(O)R^{9a}; N(R⁹)C(O)N(R^{9a}R^{9b}); N(R⁹)C(O)OR^{9a}; OC(O)N(R⁹R^{9a}); or T²;
R⁹; R^{9a}, R^{9b} are independently selected from the group consisting of H; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹², which are the same or different;
R¹⁰ is halogen; CN; C(O)OR¹³; OR¹³; oxo (═O), where the ring is at least partially saturated; C(O)R¹³; C(O)N(R¹³R^{13a}); S(O)₂N(R¹³R^{13a}); S(O)N(R¹³R^{13a}); S(O)₂R¹³; S(O)R¹³; N(R¹³)S(O)₂N(R^{13a}R^{13b}); N(R¹³)S(O)N(R^{13a}R^{13b}); SR¹³; N(R¹³R^{13a}); NO₂; OC(O)R¹³; N(R¹³)C(O)R^{13a}; N(R¹³)S(O)₂R^{13a}; N(R¹³)S(O)R^{13a}; N(R¹³)C(O)N(R^{13a}R^{13b}); N(R¹³)C(O)OR^{13a}; OC(O)N(R¹³R^{13a}); C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R¹⁴, which are the same or different;
R¹³, R^{13a}, R^{13b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹⁴, which are the same or different;
R¹¹, R¹² are independently selected from the group consisting of halogen; CN; C(O)OR¹⁵; OR¹⁵; C(O)R¹⁵; C(O)N(R¹⁵R^{15a}); S(O)₂N(R¹⁵R^{15a}); S(O)N(R¹⁵R^{15a}); S(O)₂R¹⁵; S(O)R¹⁵; N(R¹⁵)S(O)₂N(R^{15a}R^{15b}); N(R¹⁵)S(O)N(R^{15a}R^{15b}); SR¹⁵; N(R¹⁵R^{15a}); NO₂, OC(O)R¹⁵; N(R¹⁵)C(O)R^{15a}; N(R¹⁵)S(O)₂R^{15a}; N(R¹⁵)S(O)R^{15a}; N(R¹⁵)C(O)N(R^{15a}R^{15b}); N(R¹⁵)C(O)OR^{15a}; OC(O)N(R¹⁵R^{15a}); or T²;
R¹⁵, R^{15a}, R^{15b} are independently selected from the group consisting of H; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R¹⁴ is halogen; CN; C(O)OR¹⁶; OR¹⁶; C(O)R¹⁶; C(O)N(R¹⁶R^{16a}); S(O)₂N(R¹⁶R^{16a}); S(O)N(R¹⁶R^{16a}); S(O)₂R¹⁶; C(O)R¹⁶; N(R¹⁶)S(O)₂N(R^{16a}R^{16b}); N(R¹⁶)S(O)N(R^{16a}R^{16b}); SR¹⁶; N(R¹⁶R^{16a}); NO₂; OC(O)R¹⁶; N(R¹⁶)C(O)R^{16a}; N(R¹⁶)S(O)₂R^{16a}; N(R¹⁶)S(O)R^{16a}; N(R¹⁶)C(O)N(R^{16a}R^{16b}); N(R¹⁶)C(O)OR^{16a}; or OC(O)N(R¹⁶R^{16a});
R¹⁶, R^{16a}, R^{16b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T² is phenyl; naphthyl; indenyl; indanyl; C₃₋₇ cycloalkyl; 4 to 7 membered heterocyclyl; or 7 to 11 membered heterobicyclyl, wherein T² optionally substituted with one or more R¹⁷, which are the same or different;
T³ is phenyl; C₃₋₇ cycloalkyl; or 4 to 7 membered heterocyclyl; or 7 to 11 membered heterobicyclyl, wherein T³ is optionally substituted with one or more R¹⁸, which are the same or different;
R¹⁷, R¹⁸ are independently selected from the group consisting of halogen; ON; C(O)OR¹⁹; OR¹⁹; oxo (═O), where the ring is at least partially saturated; C(O)R¹⁹; C(O)N(R¹⁹R^{19a}); S(O)₂N(R¹⁹R^{19a}); S(O)N(R¹⁹R^{19a}); S(O)₂R¹⁹; S(O)R¹⁹; N(R¹⁹)S(O)₂N(R^{19a}R^{19b}); N(R¹⁹)S(O)N(R^{19a}R^{19b}); SR¹⁹; N(R¹⁹R^{19a}); NO₂; OC(O)R¹⁹; N(R¹⁹)C(O)R^{19a}; N(R¹⁹)S(O)₂R^{19a}; N(R¹⁹)S(O)R^{19a}; N(R¹⁹)C(O)N(R^{19a}R^{19b}), N(R¹⁹)C(O)OR^{19a}; OC(O)N(R¹⁹R^{19a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R²⁰, which are the same or different;
R¹⁹, R^{19a}, R^{19b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R²⁰, which are the same or different;
R²⁰ is halogen; ON; C(O)OR²¹; OR²¹; C(O)R²¹; C(O)N(R²¹R^{21a}); S(O)₂N(R²¹R^{21a}); S(O)N(R²¹R^{21a}); S(O)₂R²¹; S(O)R²¹; N(R²¹)S(O)₂N(R^{21a}R^{21b}); N(R²¹)S(O)N(R^{21a}R^{21b}); SR²¹; N(R²¹R^{21a}); NO₂; OC(O)R²¹; N(R²¹)C(O)R^{21a}; N(R²¹)S(O)₂R^{21a}; N(R²¹)S(O)R^{21a}; N(R²¹)C(O)N(R^{2la}R^{21b}); N(R²¹)C(O)OR^{21a}; or OC(O)N(R²¹R^{21a});
R²¹, R^{21a}, R^{21b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different, provided that the following compounds are excluded;

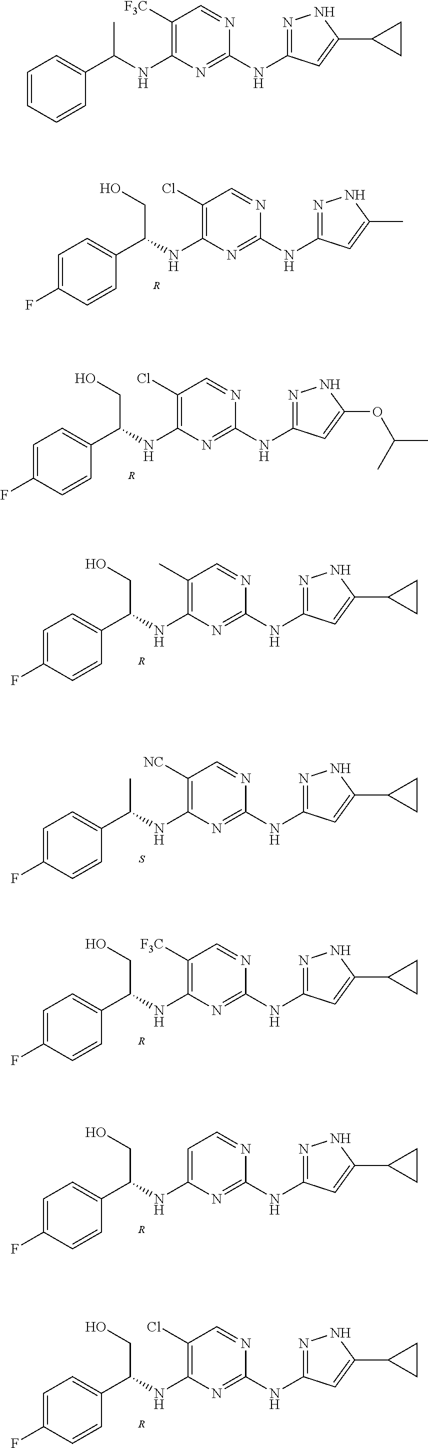

2. The compound of claim 1, wherein n is 0.

3. The compound of claim 1, wherein in formula (I) R^{Y1}, R^{Y2} and Y⁰ are defined to give formula (Ia) (Ib), (Ic), or (Id):

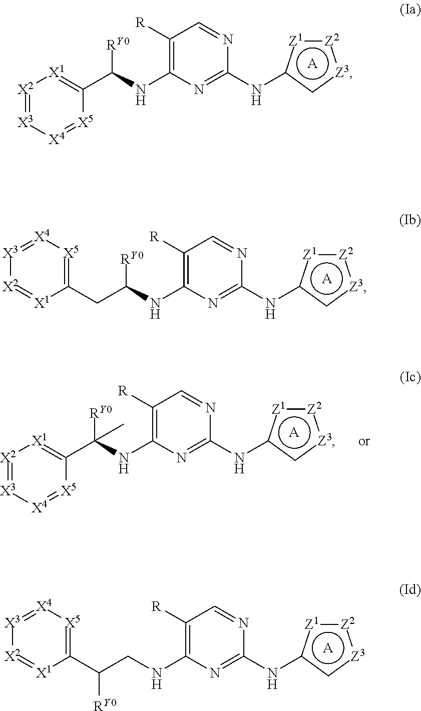

4. (canceled)

5. (canceled)

6. The compound of claim 1, wherein R^{Y0} is unsubstituted C₂₋₄ alkyl; CH₂CH₂OR^{Y5}; CH₂CH₂C(O)T^{Y1}; CH₂CH₂C(O)OR^{Y5}; CH₂CH₂OC(O)R^{Y5}; CH₂CH₂N(R^{Y5}R^{Y5}); CH₂CH₂N(R^{Y5})C(O)R^{Y5a}; CH₂CH₂C(O)N(R^{Y5}R^{Y5a}); CH₂OR^{Y5}; CH₂C(O)T^{Y1}; CH₂C(O)OR^{Y5}; CH₂OC(O)R^{Y5}, CH₂N(R^{Y5}R^{Y5a}); CH₂N(R^{Y5})C(O)R^{Y5a}; CH₂C(O)N(R^{Y5}R^{Y5a}); C(O)T^{Y1}; C(O)OR^{Y5}; or C(O)N(R^{Y5}R^{Y5a}).

7. (canceled)

8. (canceled)

9. (canceled)

10. (canceled)

11. (canceled)

12. (canceled)

13. (canceled)

14. The compound of claim 1, wherein R¹ is unsubstituted C₁₋₄ alkyl; or C₁₋₄ alkyl, substituted with one or two R³, which are the same or different,

15. (canceled)

16. (canceled)

17. (canceled)

18. (canceled)

19. (canceled)

20. The compound of claim 1, wherein R is F; Cl; CF₃; or CH₃.

21. (canceled)

22. (canceled)

23. (canceled)

24. The compound of claim 1, wherein at most three of R^{6a}, R^{6b}, R^{6C}, R^{6d} are other than H.

25. (canceled)

26. (canceled)

27. The compound of claim 1, wherein R^{6a}, R^{6b}, R^{6c}, R^{6d} are independently selected from the group consisting of H; halogen; CF₃; OR⁷; or T².

28. (canceled)

29. (canceled)

30. (canceled)

31. The compound of claim 1 or a pharmaceutically acceptable salt thereof, selected from the group consisting of
2-((5-chloro-2-((1-methyl-1H-1-pyrazol-4-yl)amino)pyrimidin-4-yl)amino)-2-phenylethanol;
2-((5-fluoro-2-((1-methyl-1H-1-pyrazol-4-yl)amino)pyrimidin-4-yl)amino)-2-phenylethanol;
(S)-2-((5-fluoro-2-((1-methyl-1H-pyrazol-4-yl)amino)pyrimidin-4-yl)amino)-2-phenylethanol;
(S)-2-((5-chloro-2-((1-methyl-1H-pyrazol-4-yl)amino)pyrimidin-4-yl)amino)-2-phenylethanol;
(R)-5-chloro-N2-(1-methyl-1H-pyrazol-4-yl)-N4-(1-phenylpropyl)pyrimidine-2,4-diamine;
(S)-2-((2-((1-methyl-1H-pyrazol-4-yl)amino)-5-(trifluoromethyl)pyrimidin-4-yl)amino)-2-phenylethanol;
2-((5-chloro-2-((1-methyl-1H-1-pyrazol-4-yl)amino)pyrimidin-4-yl)amino)-2-phenylpropan-1-ol;
2-((5-chloro-2-((1-methyl-1H-pyrazol-4-yl)amino)-5-(trifluoromethyl)pyrimidin-4-yl)amino)-2-phenylpropan-1-ol;
(S)-2-((5-chloro-2-((1-(2-hydroxyethyl)-1H-pyrazol-4-yl)amino)pyrimidin-4-yl)amino)-2-phenylethanol;
(S)-2-((5-fluoro-2-((1-(2-hydroxyethyl)-1H-pyrazol-4-yl)amino)pyrimidin-4-yl)amino)-2-phenylethanol;
(S)-2-((5-chloro-2-((1-methyl-1H-pyrazol-3-yl)amino)pyrimidin-4-yl)amino)-2-phenylethanol;
2-((2-((1-(2-hydroxyethyl)-1H-pyrazol-4-yl)amino)-5-methylpyrimidin-4-yl)amino)-2-phenylethanol;
(S)-(4-((5-chloro-4-((2-hydroxy-1-phenylethyl)amino)pyrimidin-2-yl)amino)-1H-pyrrol-2-yl)(morpholino)methanone,
2-((5-chloro-2-((1-methyl-1H-pyrazol-4-yl)amino)pyrimidin-4-yl)amino)-3-phenylpropan-1-ol;
methyl 2-((5-chloro-2-((1-(2-hydroxyethyl)-1H-pyrazol-4-yl)amino)pyrimidin-4-yl)amino)-2-phenylacetate;
methyl 2-((5-chloro-2-((1-methyl-1-yl)amino)-2-phenylacetate;
2-((5-chloro-2-((1-(2-hydroxyethyl)-1H-pyrazol-4-yl)amino)pyrimidin-4-yl)amino)-2-phenylacetic acid;
2-((5-chloro-2-((1-methyl-1H-pyrazol-4-yl)amino)pyrimidin-4-yl)amine)-2-phenylacetic add;
2-((5-chloro-2-((1-(2-hydroxyethyl)-1H-pyrazol-4-yl)amino)pyrimidin-4-yl)amino)-3-phenylpropan-1-ol;
(S)-2-((5-chloro-2-((1-methyl-1H-pyrazol-4-yl)amino)pyrimidin-4-yl)amino)-N-methyl-2-phenylacetamide;
(S)-isopropyl 2-(4-((5-chloro-4-((2-hydroxy-1-phenylethyl)amino)pyrimidin-2-yl)amino)-1H-pyrazol-1-yl)acetate;
(S)-ethyl 2-(4-((5-chloro-4-((2-hydroxy-1-phenylethyl)amino)pyrimidin-2-yl)amino)-1H-pyrazol-1-yl)acetate;
(S)-2-(4-((5-chloro-4-((2-hydroxy-1-phenylethyl)amino)pyrimidin-2-yl)amino)-1H-pyrazol-1-yl)-N-methylacetamide;
(S)-2-(4-((5-chloro-4-((2-hydroxy-1-phenylethyl)amino)pyrimidin-2-yl)amino)-1H-pyrazol-1-yl)acetic acid;
(S)-2-((5-chloro-2-((1-methyl-1H-pyrazol-4-yl)amino)pyrimidin-4-yl)amino)-N,N-dimethyl-2-phenylacetamide;
(S)-2-((5-chloro-2-((1-(2-hydroxyethyl)-1H-pyrazol-4-yl)amino)pyrimidin-4-yl)amino)-N,N-dimethyl-2-phenylacetamide;
(S)-2-((5-chloro-2-((1-(2-hydroxyethyl)-1H-pyrazol-4-yl)amino)pyrimidin-4-yl)amino)-N-ethyl-2-phenylacetamide;
(S)-2-((5-chloro-2-((1-methyl-1H-1-pyrazol-4-yl)amino)pyrimidin-4-yl)amino)-N-ethyl-2-phenylacetamide;
(S)-2-((5-chloro-2-((1-methyl-1H-pyrazol-4-yl)amino)pyrimidin-4-yl)amino)-N-(2-methoxyethyl)-2-phenylacetamide;
(S)-2-(4-((5-chloro-4-((2-hydroxy-1-phenylethyl)amino)pyrimidin-2-yl)amino)-1H-pyrazol-1-yl)-N,N-dimethylacetamide;
2-(4-((5-chloro-4-((1-(2,6-difluorophenyl)-2-hydroxyethyl)amino)pyrimidin-2-yl)amino)-1H-pyrazol-1-yl)-N-methylacetamide;
2-(4-((5-chloro-4-((1-(3-fluorophenyl)-2-hydroxyethyl)amino)pyrimidin-2-yl)amino)-1H-pyrazol-1-yl)-N-methylacetamide;
2-(4-((5-chloro-4-((1-(2-fluorophenyl)-2-hydroxyethyl)amino)pyrimidin-2-yl)amino)-1H-pyrazol-1-yl)-N-methylacetamide;
(S)-2-((5-chloro-2-((1-methyl-1H-pyrazol-4-yl)amino)pyrimidin-4-yl)amino)-2-phenyl-1-(pyrrolidin-1-yl)ethanone;
(S)-2-((5-chloro-2-((1-(2-hydroxyethyl)-1H-pyrazol-4-yl)amino)pyrimidin 4-yl)amino)-2-phenyl-1-(pyrrolidin-1-yl)ethanone;
2-((5-chloro-2-((1-(2-hydroxyethyl)-1H-pyrazol-4-yl)amino)pyrimidin-4-yl)amino)-2-(2-fluorophenyl)ethanol;
2-((5-chloro-2-((1-methyl-1H-1-pyrazol-4-yl)amino)pyrimidin-4-yl)amino)-2-(2-fluorophenyl)ethanol;
1-(4-((5-chloro-4-((1-(3-fluorophenyl)-2-hydroxyethyl)amino)pyrimidin-2-yl)amino)-1H-pyrazol-1-yl)propan-2-ol;
2-((5-chloro-2-((1-(2-hydroxyethyl)-1H-pyrazol-4-11)amino)pyrimidin-4-yl)amino)-2-(3-fluorophenyl)ethanol;
2-((5-chloro-2-((1-methyl-1H-pyrazol-4-yl)amino)pyrimidin-4-yl)amino)-2-(3-fluorophenyl)ethanol;
2-((5-chloro-2-((1-(2-hydroxyethyl)-1H-pyrazol-3-yl)amino pyrimidin-4-yl)amino)-2-(3-fluorophenyl)ethanol;
1-(4-((5-chloro-4-((1-(2,6-difluorophenyl)-2-hydroxyethyl)amino)pyrimidin-2-yl)amino)-1H-pyrazol-1-yl)propan-2-ol;
2-((5-chloro-2-((1-(2-hydroxyethyl)-1H-pyrazol-4-yl)amino)pyrimidin-4-yl)amino)-2-(2,6-difluorophenyl)ethanol;
2-((5-chloro-2-((1-methyl-1H-pyrazol-4-yl)amino)pyrimidin-4-yl)amino)-2-(2,6-difluorophenyl)ethanol;
2-((5-chloro-2-((1-(2-hydroxyethyl)-1H-pyrazol-3-yl)amino)pyrimidin-4-yl)amino)-2-(2,6-difluorophenyl)ethanol;
(S)-2-((5-chloro-2-((1-methyl-1H-pyrazol-4-yl)amino)pyrimidin-4-yl)amino)-N-(2-hydroxyethyl)-2-phenylacetamide;
(S)-2-((5-chloro-2-((1-(2-hydroxyethyl)-1H-pyrazol-4-yl)amino)pyrindin-4-yl)amino)-N-(2-hydroxyethyl)-2-phenylacetamide;
(S)-2-((5-chloro-2-((1-(2-hydroxyethyl)-1H-pyrazol-4-yl)amino)pyrimidin-4-yl)amino)-N-(2-hydroxyethyl)-2-phenylacetamide;
(S)-2-((5-chloro-2-((1-methyl-1H-pyrazol-4-yl)amino)pyrimidin-4-yl)amino)-2-phenyl-1-(piperidin-1-yl)ethanone:
2-((5-chloro-2-((1-(2-hydroxyethyl)-1H-pyrazol-3-yl)amino)pyrimidin-4-yl)amino)-2-(2-fluorophenyl)ethanol:
(S)-2-((5-chloro-2-((1-(2-hydroxyethyl)-1H-pyrazol-4-yl)amino)pyrimidin-4-yl)amino)-2-phenyl-1-(piperidin-1-yl)ethanone;
2-(3-(2-oxa-6-azaspiro[3.3]heptan-6-yl)phenyl)-2-((5-chloro-2-((1-methyl-1H-pyrazol-4-yl)amino)pyrimidin-4-yl)amino)ethanol;
(S)-5-chloro-N4-(2-methoxy-1-phenylethyl)-N2-(1-methyl-1H-pyrazol-4-yl)pyrimidine-2,4-diamine;
(S)-2-(4-((5-chloro-4-((2-hydroxy-1-phenylethyl)amino)pyrimidin-2-yl)amino)-1H-pyrazol-1-yl)acetamide;
1-(4-((5-chloro-4-((1-(2-fluorophenyl)-2-hydroxyethyl)amino)pyrimidin-2-yl)amino)-1H-pyrazol-1-yl)-2-methylpropan-2-ol;
(S)-2-((5-chloro-2-((1-methyl-1H-pyrazol-4-yl)amino)pyrimidin-4-yl)amino)-2-(2-fluorophenyl)ethanol;
(S)-2-((5-chloro-2-((1-methyl-1H-pyrazol-4-yl)amino)pyrimidin-4-yl)amino)-N-cyclopentyl-2-phenylacetamide;
(S)-2-((5-chloro-2-((1-(2-hydroxyethyl)-1H-pyrazol-4-yl)amino)pyrimidin-4-yl)amino)-N-cyclopentyl-2-phenylacetamide;
1-(4-((5-chloro-4-((1-(3-fluorophenyl)-2-hydroxyethyl)amino)pyrimidin-2-yl)amino)-1H-pyrazol-1-yl)-2-methylpropan-2-ol;
1-(4-((5-chloro-4-((1-(2,6-difluorophenyl)-2-hydroxyethyl)amino)pyrimidin-2-yl)amino)-1H-pyrazol-1-yl)-2-methylpropan-2-ol;
3-(4-((5-chloro-4-((1-(2-fluorophenyl)-2-hydroxyethyl)amino)pyrimidin-2-yl)amino)-1H-pyrazol-1-yl)propan-1-ol:
(R)-3-((5-chloro-2-((1-(2-hydroxyethyl)-1H-pyrazol-4-yl)amino)pyrimidin-4-yl)amino)-3-phenylpropan-1-ol;
(R)-2-(4-((5-chloro-4-((3-hydroxy-1-phenylpropyl)amino)pyrimidin-2-yl)amino)-1H-pyrazol-1-yl)-N-methylacetamide;
(S)-2-(4-((5-chloro-4-((2-hydroxy-1-phenylethyl)amino)pyrimidin-2-yl)amino)-1H-pyrazol-1-yl)-N-isopropylacetamide;
(S)-2-(4-((5-chloro-4-((2-hydroxy-1-phenylethyl)amino)pyrimidin-2-yl)amino)-1H-pyrazol-1-yl)-N-cyclopropylacetamide;
2-((5-chloro-2-((1-methyl-1H-pyrazol-4-yl)amino)pyrimidin-4-yl)amino)-2-(3-(1-methyl-1H-pyrazol-4-yl)phenyl)ethanol, (S)-2-(4-((5-chloro-4-((2-hydroxy-1-phenylethyl)amino)pyrimidin-2-yl)amino)-1H-pyrazol-1-yl)-N-(cyanomethyl)-N-methylacetamide;
2-((5-chloro-2-((1-methyl-1H-pyrazol-4-yl)amino)pyrimidin-4-yl)amino)-2-(4-fluorophenyl)ethanol;
2-((5-chloro-2-((1-(2-hydroxyethyl)-1H-pyrazol-4-yl)amino)pyrimidin-4-yl)amino)-2-(4-fluorophenyl)ethanol;
(S)-2-(4-((5-chloro-4-((1-(2-fluorophenyl)-2-hydroxyethyl)amino)pyrimidin-2-yl)amino)-1H-pyrazol-1-yl)-2-methylpropanamide;
(S)-2-((5-chloro-2-((1-methyl-1H-pyrazol-4-yl)amino)pyrimidin-4-yl)amino)-2-(2-methoxyphenyl)ethanol;
(S)-2-((2-((1H-pyrazol-4-yl)amino)-5-chloropyrimidin-4-yl)amino)-2-phenylethanol;
(S)-2-((5-chloro-2-((1-(2-hydroxyethyl)-1H-pyrazol-4-yl)amino)pyrimidin-4-yl)amino)-2-(2-methoxyphenyl)ethanol;
(S)-2-((5-chloro-2-((1-(2-hydroxyethyl)-1H-pyrazol-4-yl)amino)pyrimidin-4-yl)amino)-2-(2-fluorophenyl)ethanol;
(S)-2-(4-((5-chloro-4-((1-(2-fluorophenyl)-2-hydroxyethyl)amino)pyrimidin-2-yl)amino)-1H-pyrazol-1-yl)acetamide;
(S)-2-((2-((1H-pyrazol-4-yl)amino)-5-chloropyrimidin-4-yl)amino)-2-(2-fluorophenyl)ethanol;
(S)-2-((5-chloro-2-((1-(2-hydroxyethyl)-1H-pyrazol-4-yl)amino)pyrimidin-4-yl)amino)-2-(pyridin-2-yl)ethanol;
(S)-2-(4-((5-chloro-4-((2-hydroxy-1-(pyridin-2-yl)ethyl)amino)pyrimidin-2-yl)amino)-1H-pyrazol-1-yl)acetamide;
(S)-2-((5-chloro-2-((1-methyl-1H-pyrazol-4-yl)amino)pyrimidin-4-yl)amino)-2-(3-methoxyphenyl)ethanol;
(S)-2-((5-chloro-2-((1-(2-hydroxyethyl)-1H-pyrazol-4-yl)amino)pyrimidin-4-yl)amino)-2-(3-methoxyphenyl)ethanol;
(S)-2-(4-((5-chloro-4-((2-hydroxy-1-(3-methoxyphenyl)ethyl)amino)pyrimidin-2-yl)amino)-1H-pyrazol-1-yl)acetamide;
(S)-2-((5-chloro-2-((1-methyl-1H-pyrazol-4-yl)amino)pyrimidin-4-yl)amino)-2-(3-fluorophenyl)ethanol;
(S)-2-((5-chloro-2-((1-(2-hydroxyethyl)-1H-pyrazol-4-yl)amino)pyrimidin-4-yl)amino)-2-(3-fluorophenyl)ethanol;
(S)-2-(4-((5-chloro-4-((1-(3-fluorophenyl)-2-hydroxyethyl)amino)pyrimidin-2-yl)amino)-1H-pyrazol-1-yl)acetamide,
(S)-2-((2-((1H-pyrazol-4-yl)amino)-5-chloropyrimidin-4-yl)amino)-2-(3-fluorophenyl)ethanol;
(S)-2-(4-((5-chloro-4-(2-hydroxy-1-phenylethyl)amino)pyrimidin-2-yl)amino)-1H-pyrazol-1-yl)-N-(2-(dimethylamino)ethyl)acetamide;
(S)-2-(4-((5-chloro-4-((2-hydroxy-1-phenylethyl)amino)pyrimidin-2-yl)amino)-1H-pyrazol-1-yl)-N-(3-(dimethylamino)propyl)acetamide;
(S)-2-((5-chloro-2-((1-methyl-1H-pyrazol-4-yl)amino)pyrimidin-4-yl)amino)-2-(pyridin-3-yl)ethanol;
(S)-2-((5-chloro-2-((1-(2-hydroxyethyl)-1H-pyrazol-4-yl)amino)pyrimidin-4-yl)amino)-2-(pyridin-3-yl)ethanol:
2-(3-bromophenyl)-2-(4-chloro-2-((1-(2-hydroxyethyl)-1H-pyrazol-4-yl)amino)pyrimidin-4-yl)amino)ethanol;
2-((5-chloro-2-((1-(2-hydroxyethyl)-1H-pyrazol-4-yl)amino)pyrimidin-4-yl)amino)-2-(3-(1-methyl-1H-pyrazol-4-yl)phenyl)ethanol;
(S)-2-((5-chloro-2-((1-ethyl-1H-pyrazol-4-yl)amino)pyrimidin-4-yl)amino)-2-(2-methoxyphenyl)ethanol;
(S)-2-((5-chloro-2-((1-ethyl-1H-pyrazol-4-yl)amino)pyrimidin-4-yl)amino)-2-(3-methoxyphenyl)ethanol;
(S)-2-((5-chloro-2-((1-ethyl-1H-pyrazol-4-yl)amino)pyrimidin-4-yl)amino)-2-(pyridin-3-yl)ethanol;
(S)-2-(4-((5-chloro-4-((2-hydroxy-1-(2-methoxyphenyl)ethyl)amino)pyrimidin-2-yl)amino)-1H-pyrazol-1-yl)-N-methylacetamide:
(S)-2-(4-((5-chloro-4-((2-hydroxy-1-(3-methoxyphenyl)ethyl)amino)pyrimidin-2-yl)amino)-1H-pyrazol-1-0)-N-methylacetamide;
(S)-2-(4-((5-chloro-4-(1-(2-fluorophenyl)-2-hydroxyethyl)amino)pyrimidin-2-yl)amino)-1H-pyrazol-1-yl)-N-methylacetamide;
(S)-2-(4-((5-chloro-4-((2-hydroxy-1-(2-methoxyphenyl)ethyl)amino)pyrimidin-2-yl)amino)-1H-pyrazol-1-yl)-1-morpholinoethanone;
(S)-2-(4-((5-chloro-((2-hydroxy-1-(3-methoxyphenyl)ethyl)amino)pyrimidin-2-yl)amino)-1H-pyrazol-1-yl)-1-morpholinoethanone;
(S)-2-(4-((5-chloro-4-((1-(2-fluorophenyl)-2-hydroxyethyl)amino)pyrimidin-2-yl)amino)-1H-pyrazol-1-yl)-1-morpholinoethanone;
(S)-2-(4-((5-chloro-4-((2-hydroxy-1-(pyridin-3-yl)ethyl)amino)pyrimidin-2-yl)amino)-1H-pyrazol-1-yl)-1-morpholinoethanone:
(S)-2-((5-chloro-2-((1-Isopropyl-1H-pyrazol-4-yl)amino)pyrimidin-4-yl)amino)-2-(2-methoxyphenyl)ethanol;
(S)-2-((5-chloro-2-((1-isopropyl-1H-pyrazol-4-yl)amino)pyrimidin-4-yl)amino)-2-(3-methoxyphenyl)ethanol;
(S)-2-((5-chloro-2-((1-isopropyl-1H-pyrazol-4-yl)amino)pyrimidin-4-yl)amino)-2-(pyridin-3-yl)ethanol;
(S)-2-(4-((5-fluoro-4-((2-hydroxy-1-phenylethyl)amino)pyrimidin-2-yl)amino)-1H-pyrazol-1-yl)acetamide;
(S)-2-((5-chloro-2-((1-ethyl-1H-pyrazol-4-yl)amino)pyrimidin-4-yl)amino)-2-(2-fluorophenyl)ethanol;
(S)-2-((5-chloro-2-(0-isopropyl-1H-pyrazol-4-yl)amino)pyrimidin-4-yl)amino)-2-(2-fluorophenyl)ethanol,
(S)-2-(4-((5-fluoro-4-((2-hydroxy-1-phenylethyl)amino)pyrimidin-2-yl)amino)-1H-pyrazol-1-yl)-N-methylacetamide,
(S)-2-(4-((5-fluoro-4-((2-hydroxy-1-phenylethyl)amino)pyrimidin-2-yl)amino)-1H-pyrazol-1-morpholinoethanone;
(S)-2-((2-(0-(3-aminopropyl)-1H-pyrazol-4-yl)amino)-5-chloropyrimidin-4-yl)amino)-2-phenylethanol;
(S)-2-((5-chloro-2-((1-(2-isopropoxyethyl)-1H-pyrazol-4-yl)amino)pyrimidin-4-yl)amino)-2-phenylethanol;
(S)-2-(4-((5-chloro-4-((1-(2-fluorophenyl)-2-hydroxyethyl)amino)pyrimidin-2-yl)amino)-1H-pyrazol-1-yl)-N-isopropylacetamide;
2-(4-((5-chloro-4-(((S)-1-(2-fluorophenyl)-2-hydroxyethyl)amino)pyrimidin-2-yl)amino)-1H-pyrazol-1-yl)-N-((S)-1-hydroxypropan-2-yl)acetamide;
2-((5-chloro-2-((1-methyl-1H-pyrazol-4-yl)amino)pyrimidin-4-yl)amino)-2-(2-chlorophenyl)ethanol;
2-(4-((5-chloro-4-((1-(2-chlorophenyl)-2-hydroxyethyl)amino)pyrimidin-2-yl)amino)-1H-pyrazol-1-yl)acetamide;
2-(4-((5-chloro-4-((1-(2-chlorophenyl)-2-hydroxyethyl)amino)pyrimidin-2-yl)amino)-1H-pyrazol-1-yl)-N-methylacetamide;
2-((5-chloro-2-((1-(2-hydroxyethyl)-1H-pyrazol-4-yl)amino)pyrimidin-4-yl)amino)-2-(2-chlorophenyl)ethanol;
2-((5-chloro-2-((1-methyl-1H-pyrazol-4-yl)amino)pyrimidin-4-yl)amino)-2-(3,5-difluorophenyl)ethanol;
2-((5-chloro-2-((1-(2-hydroxyethyl)-1H-pyrazol-4-yl)amino)pyrimidin-4-yl)amino)-2-(3,5-difluorophenyl)ethanol:
2-(4-((5-chloro-4-((1-(3,5-difluorophenyl)-2-hydroxyethyl)amino)pyrimidin-2-yl)amino)-1H-pyrazol-1-yl)-N-methylacetamide;
4-(3-(1-((5-chloro-2-((1-methyl-1H-pyrazol-4-yl)amino)pyrimidin-4-yl)amino)-2-hydroxyethyl)phenyl)morpholin-3-one;
5-chloro-N4-(1-(2-fluorophenyl)propyl)-N2-(1-methyl-1H-pyrazol-4-yl)pyrimidine-2,4-diamine;
2-(4-((5-chloro-4-((1-(2-fluorophenyl)propylamino)pyrimidin-2-yl)amino)-1H-pyrazol-1-yl)acetamide;
2-((5-chloro-2-((1-(2-hydroxyethyl)-1H-pyrazol-4-yl)amino)pyrimidin-4-yl)amino)-2-(2-fluorophenyl)-N,N-dimethylacetamide;
2-((5-chloro-2-((1-(2-hydroxyethyl)-1H-pyrazol-4-yl)amino)pyrimidin-4-yl)amino)-2-(2-fluorophenyl)-N-(2,2,2-trifluoroethyl)acetamide;
2-((5-chloro-2-((1-methyl-1H-pyrazol-4-yl)amino)pyrimidin-4-yl)amino)-2-(2,5-difluorophenyl)ethanol;
2-(4-((5-chloro-4-((1-(2,5-difluorophenyl)-2-hydroxyethyl)amino)pyrimidin-2-yl)amino)-1H-pyrazol-1-yl)-N-methylacetamide;
2-(4-((5-chloro-4-((2-hydroxy-1-(2-(trifluoromethyl)phenyl)ethyl)amino)pyrimidin-2-yl)amino)-1H-pyrazol-1-yl)-N-methylacetamide;
2-(4-((5-chloro-4-((1-(2,5-difluorophenyl)-2-hydroxyethyl)amino)pyrimidin-2-yl)amino)-1H-pyrazol-1-yl)acetamide;
(S)-2-(4-((4-((2-hydroxy-1-phenylethyl)amino)-5-methylpyrimidin-2-yl)amino)-1H-pyrazol-1-yl)-N-methylacetamide;
(S)-2-(4-((4-((2-hydroxy-1-phenylethyl)amino)-5-methylpyrimidin-2-yl)amino)-1H-pyrazol-1-yl)-N,N-dimethylacetamide;
3-(4-((5-chloro-4-(((S)-2-hydroxy-1-phenylethyl)amino)pyrimidin-2-yl)amino)-1H-pyrazol-1-yl)-1-methylpyrrolidin-2-one;
(S)-2-((5-chloro-2-((1-methyl-1H-pyrazol-4-yl)amino)pyrimidin-4-yl)amino)-2-(3-(1-methyl-1H-pyrazol-4-yl)phenyl)ethanol;
(S)-2-((5-chloro-2-((1-methyl-1H-pyrazol-4-yl)amino)pyrimidin-4-yl)amino)-2-(3-fluoro-5-(1-methyl-1H-1-pyrazol-4-yl)phenyl)ethanol;
(S)-4-(3-((5-chloro-2-((1-methyl-1H-pyrazol-4-yl)amino)pyrimidin-4-yl)amino)-2-hydroxyethyl)phenyl)morpholin-3-one;
(S)-2-((5-chloro-2-((1-methyl-1H-pyrazol-4-yl)amino)pyrimidin-4-yl)amino)-2-(2,5-difluorophenyl)ethanol;
(S)-2-(4-((5-chloro-4-((1-(2,5-difluorophenyl)-2-hydroxyethyl)amino)pyrimidin-2-yl)amino)-1H-pyrazol-1-yl)acetamide;
(S)-2-(4-((5-chloro-4-((1-(2,5-difluorophenyl)-2-hydroxyethyl)amino)pyrimidin-2-yl)amino)-1H-pyrazol-1-yl)-N-methylacetamide;
(S)-2-((5-chloro-2-((1-methyl-1H-pyrazol-4-yl)amino)pyrimidin-4-yl)amino)-2-(3,5-difluorophenyl)ethanol;
(S)-2-((5-chloro-2-((1-(2-hydroxyethyl)-1H-pyrazol-4-yl)amino)pyrimidin-4-yl)amino)-2-(3,5-difluorophenyl)ethanol;
(S)-2-(4-((5-chloro-4-((1-(3,5-difluorophenyl)-2-hydroxyethyl)amino)pyrimidin-2-yl)amino)-1H-pyrazol-1-yl)acetamide;
(S)-2-(4-((5-chloro-4-((1-(3,5-difluorophenyl)-2-hydroxyethyl)amino)pyrimidin-2-yl)amino)-1H-pyrazol-1-yl)-N-methylacetamide;
(S)-2-(2,5-difluorophenyl)-2-((5-fluoro-2-((1-methyl-1H-pyrazol-4-yl)amino)pyrimidin-4-yl)amino)ethanol;
(S)-2-(2,5-difluorophenyl)-2-((5-fluoro-2-((1-(2-hydroxyethyl)-1H-pyrazol-4-yl)amino)pyrimidin-4-yl)amino)ethanol;
(S)-2-(4-((4-(1-(2,5-difluorophenyl)-2-hydroxyethyl)amino)-5-fluoropyrimidin-2-yl)amino)-1H-pyrazol-1-yl)-N-methylacetamide;
(S)-2-(3-(1-methyl-1H-pyrazol-4-yl)phenyl)-2-((5-methyl-2-((1-methyl-1H-pyrazol-4-yl)amino)pyrimidin-4-yl)amino)ethanol;
(S)-2-((5-fluoro-2-((1-methyl-1H-pyrazol-4-yl)amino)pyrimidin-4-yl)amino)-2-(3-fluoro-5-(1-methyl-1H-pyrazol-4-yl)phenyl)ethanol;
(S)-2-((5-chloro-2-((1-(2-hydroxyethyl)-1H-pyrazol-4-yl)amino)pyrimidin-4-yl)amino)-2-(3-(1-methyl-1H-pyrazol-4-yl)phenyl)ethanol;
(S)-2-((5-chloro-2-((1-(2-hydroxyethyl)-1H-pyrazol-4-yl)amino)pyrimidin-4-yl)amino)-2-(3-fluoro-5-(1-methyl-1H-pyrazol-4-yl)phenyl)ethanol,
(S)-2-((5-fluoro-2-((1-(2-hydroxyethyl)-1H-pyrazol-4-yl)amino)pyrimidin-4-yl)amino)-2-(3-fluoro-5-(1-methyl-1H-pyrazol-4-yl)phenyl)ethanol;
(S)-2-(4-((5-chloro-4-((2-hydroxy-1-(3-(1-methyl-1H-pyrazol-4-yl)phenyl)ethyl)amino)pyrimidin-2-yl)amino)-1H-pyrazol-1-yl)-N-methylacetamide;
(S)-2-(4-((5-fluoro-4-((2-hydroxy-1-(3-(1-methyl-1H-pyrazol-4-yl)phenyl)ethyl)amino)pyrimidin-2-yl)amino)-1H-pyrazol-1-yl)-N-methylacetamide;
(S)-2-(4-((5-chloro-4-((1-(3-fluoro-5-(1-methyl-1H-pyrazol-4-yl)phenyl)-2-hydroxyethyl)amino)pyrimidin-2-yl)amino)-1H-pyrazol-1-yl)-N-methylacetamide;
(S)-2-(4-((5-fluoro-4-((1-(3-fluoro-5-(1-methyl-1H-pyrazol-1-yl)phenyl)-2-hydroxyethyl)amino)pyrimidin-2-yl)amino)-1H-pyrazol-1-yl)-N-methylacetamide:
(S)-2-(3,5-difluorophenyl)-2-((5-fluoro-2-((1-methyl-1H-pyrazol-4-yl)amino)pyrimidin-4-yl)amino)ethanol;
(S)-2-(3,5-difluorophenyl)-2-((5-fluoro-2-((1-(2-hydroxyethyl)-1H-pyrazol-4-yl)amino)pyrimidin-4-yl)amino)ethanol:
(S)-2-(4-((4-((1-(3,5-difluorophenyl)-2-hydroxyethyl)amino)-5-fluoropyrimidin-2-yl)amino)-1H-pyrazol-1-yl)acetamide;
(S)-2-(4-((4-((1-(3,5-difluorophenyl)-2-hydroxyethyl)amino)-5-fluoropyrimidin-2-yl)amino)-1H-pyrazol-1-yl)-N-methylacetamide;
(S)-4-(3-(1-((5-chloro-2-((1-methyl-1H-pyrazol-4-yl)amino)pyrimidin-4-yl)amino)-2-hydroxyethyl)-2-fluorophenyl)morpholin-3-one;
(S)-2-(2,5-difluorophenyl)-2-((5-methyl-2-((1-methyl-1H-pyrazol-4-yl)amino)pyrimidin-4-yl)amino)ethanol;
(S)-2-(3,5-difluorophenyl)-2-((5-methyl-2-((1-methyl-1H-pyrazol-4-yl)amino)pyrimidin-4-yl)amino)ethanol;
(S)-2-((5-chloro-2-((1-(2,2-difluoroethyl)-1H-pyrazol-4-yl)amino)pyrimidin-4-yl)amino)-2-(2-fluorophenyl)ethanol,
(S)-2-((2-((1-(2,2-difluoroethyl)-1H-pyrazol-4-yl)amino)-5-methylpyrimidin-4-yl)amino)-2-(2,5-difluorophenyl)ethanol:
(S)-2-((2-((1-(2,2-difluoroethyl)-1H-pyrazol-4-yl)amino)-5-methylpyrimidin-4-yl)amino)-2-(3,5-difluorophenyl)ethanol;
2-((2-((1-(2,2-difluoroethyl)-1H-pyrazol-4-yl)amino)-5-methylpyrimidin-4-yl)amino)-2-(2,6-difluorophenyl)ethanol;
2-(4-((4-((1-2,6-difluorophenyl)-2-hydroxyethyl)amino)-5-methylpyrimidin-2-yl)amino)-1H-pyrazol-1-yl)-1-morpholinoethanone;
(S)-2-(4-((5-chloro-4-((2-hydroxy-1-(pyridin-3-yl)ethyl)amino)pyrimidin-2-yl)amino)-1H-pyrazol-1-yl)-N-methylacetamide;
(S)-2-(3-bromophenyl)-2-((5-chloro-2-((1-methyl-1H-pyrazol-4-yl)amino)pyrimidin-4-yl)amino)ethanol;
(S)-2-(3-bromo-5-fluorophenyl)-2-((5-chloro-2-((1-methyl-1H-pyrazol-4-yl)amino)pyrimidin-4-yl)amino)ethanol;
(S)-2-(3-bromophenyl)-2-((5-methyl-2-((1-methyl-1H-pyrazol-4-yl)amino)pyrimidin-4-yl)amino)ethanol;
(S)-2-(3-bromo-5-fluorophenyl)-2-((5-fluoro-2-((1-methyl-1H-pyrazol-4-yl)amino)pyrimidin-4-yl)amino)ethanol;
(S)-2-(3-bromophenyl)-2-((5-chloro-2-((1-(2-hydroxyethyl)-1H-pyrazol-4-yl)amino)pyrimidin-4-0)amino)ethanol;
(S)-2-(3-bromo-5-fluorophenyl)-2-((5-chloro-2-((1-(2-hydroxyethyl)-1H-pyrazol-4-yl)amino)pyrimidin-4-yl)amino)ethanol;
(S)-2-(3-bromo-5-fluorophenyl)-2-((5-fluoro-2-((1-(2-hydroxyethyl)-1H-pyrazol-4-yl)amino)pyrimidin-4-yl)amino)ethanol;
(S)-2-(4-((4-((1-(3-bromophenyl)-2-hydroxyethyl)amino)-5-chloropyrimidin-2-yl)amino)-1H-pyrazol-1-yl)-N-methylacetamide;
(S)-2-(4-((4-((1-(3-bromophenyl)-2-hydroxyethyl)amino)-5-fluoropyrimidin-2-yl)amino)-1H-pyrazol-1-yl)-N-methylacetamide;
(S)-2-(4-((4-((1-(3-bromo-5-fluorophenyl)-2-hydroxyethyl)amino)-5-chloropyrimidin-2-yl)amino)-1H-pyrazol-1-yl)-N-methylacetamide;
(S)-2-(4-((4-((1-(3-bromo-5-fluorophenyl)-2-hydroxyethyl)amino)-5-fluoropyrimidin-2-yl)amino)-1H-pyrazol-1-yl)-N-methylacetamide; and
2-(3-bromophenyl)-2-((5-chloro-2-((1-methyl-1H-pyrazol-4-yl)amino)pyrimidin-4-yl)amino)ethanol.

32. The compound of claim 1 or a pharmaceutically acceptable salt thereof which is (S)-2-(4-((5-chloro-4-((2-hydroxy-1-phenylethyl)amino)pyrimidin-2-yl)amino)-1H-pyrazol-1-yl)-N-methylacetamide:

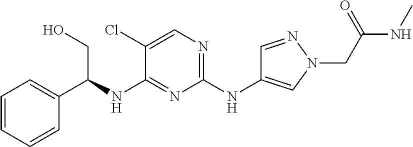 [SMILES: CNC(=O)Cn1cc(Nc2ncc(Cl)c(N[C@H](CO)c3ccccc3)n2)cn1]

33. The compound of claim 1 or a pharmaceutically acceptable salt thereof which is (S)-2-(4-((5-chloro-4-((1-(2-fluorophenyl)-2-hydroxyethyl)amino)pyrimidin-2-yl)amino-1H-pyrazol-1-yl)acetamide:

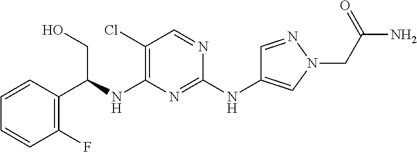 [SMILES: NC(=O)Cn1cc(Nc2ncc(Cl)c(N[C@H](CO)c3ccccc3F)n2)cn1]

34. A pharmaceutical composition comprising a compound or a pharmaceutically acceptable salt thereof of claim 1 together with a pharmaceutically acceptable carrier, optionally in combination with one or more other pharmaceutical compositions.

35. (canceled)

36. A method of treating or preventing a disease or disorder associated with JAK in a mammalian patient in need thereof, where the method comprises the administration to said patient a therapeutically effective amount of a compound of claim 1 or pharmaceutically acceptable salt thereof.

37. A method of treating or preventing an immunological, inflammatory, autoimmune, or allergic disorder or disease of a transplant rejection or a Graft-versus host disease in a mammalian patient in need thereof, where the method comprises the administration to said patient a therapeutically effective amount of a compound of claim 1 or pharmaceutically acceptable salt thereof.

38. A method of treating or preventing a proliferative disease in a mammalian patient in need thereof, where the method comprises the administration to said patient a therapeutically effective amount of a compound of claim 1 or pharmaceutically acceptable salt thereof.

39. (canceled)

40. A method for treating, controlling, delaying or preventing in a mammalian patient in need thereof one or more conditions selected from the group consisting of diseases and disorders associated with JAK, wherein the method comprises the administration to said patient a therapeutically effective amount of a compound of any claim 1 or a pharmaceutically acceptable salt thereof.
